SEQUENCE LISTING (91 sequences)

SEQ ID NO: 1 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 2 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 3 - protein, 1291 aa, Clostridium botulinum
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 4 - protein, 1276 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 5 - protein, 1252 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 6 - protein, 1278 aa, Clostridium botulinum
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 7 - protein, 1297 aa, Clostridium botulinum
MPVNIKNFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 8 - protein, 1315 aa, Clostridium tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 9 - protein, 1315 aa, synthetic construct
  REGION 1..1315: note = BoNT/AB
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 10 - protein, 1327 aa, synthetic construct
  REGION 1..1327: note = BoNT/AB
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIIELGGGGSELSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 11 - protein, 1314 aa, synthetic construct
  REGION 1..1314: note = BoNT/AB
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 12 - protein, 1304 aa, synthetic construct
  REGION 1..1304: note = BoNT/AB
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 13 - protein, 1304 aa, synthetic construct
  REGION 1..1304: note = BoNT/AB
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 14 - protein, 6 aa, synthetic construct
  REGION 1..6: note = artificial
NIINTS

SEQ ID NO: 15 - protein, 6 aa, synthetic construct
  REGION 1..6: note = artificial
NIVNTS

SEQ ID NO: 16 - protein, 6 aa, synthetic construct
  REGION 1..6: note = artificial
NIVNTS

SEQ ID NO: 17 - protein, 6 aa, synthetic construct
  REGION 1..6: note = artificial
NITNAS

SEQ ID NO: 18 - protein, 6 aa, synthetic construct
  REGION 1..6: note = artificial
NIINTS

SEQ ID NO: 19 - protein, 6 aa, synthetic construct
  REGION 1..6: note = artificial
NIINTS

SEQ ID NO: 20 - protein, 6 aa, synthetic construct
  REGION 1..6: note = artificial
NIINTS

SEQ ID NO: 21 - protein, 6 aa, synthetic construct
  REGION 1..6: note = artificial
NITNTS

SEQ ID NO: 22 - protein, 6 aa, synthetic construct
  REGION 1..6: note = artificial
EILNNI

SEQ ID NO: 23 - protein, 6 aa, synthetic construct
  REGION 1..6: note = artificial
EILNNI

SEQ ID NO: 24 - protein, 6 aa, synthetic construct
  REGION 1..6: note = artificial
EILNNI

SEQ ID NO: 25 - protein, 6 aa, synthetic construct
  REGION 1..6: note = artificial
EILNNI

SEQ ID NO: 26 - protein, 6 aa, synthetic construct
  REGION 1..6: note = artificial
DILNNI

SEQ ID NO: 27 - protein, 6 aa, synthetic construct
  REGION 1..6: note = artificial
EILNNI

SEQ ID NO: 28 - protein, 6 aa, synthetic construct
  REGION 1..6: note = artificial
EILNNI

SEQ ID NO: 29 - protein, 6 aa, synthetic construct
  REGION 1..6: note = artificial
EILNNI

SEQ ID NO: 30 - protein, 6 aa, synthetic construct
  REGION 1..6: note = artificial
NINDSK

SEQ ID NO: 31 - protein, 6 aa, synthetic construct
  REGION 1..6: note = artificial
SINDSK

SEQ ID NO: 32 - protein, 6 aa, synthetic construct
  REGION 1..6: note = artificial
SINDSK

SEQ ID NO: 33 - protein, 6 aa, synthetic construct
  REGION 1..6: note = artificial
SINDSK

SEQ ID NO: 34 - protein, 6 aa, synthetic construct
  REGION 1..6: note = arttificial
RIKSSS

SEQ ID NO: 35 - protein, 6 aa, synthetic construct
  REGION 1..6: note = artificial
RIKSSS

SEQ ID NO: 36 - protein, 6 aa, synthetic construct
  REGION 1..6: note = artificial
RIKSSS

SEQ ID NO: 37 - protein, 6 aa, synthetic construct
  REGION 1..6: note = artificial
RIKSSS

SEQ ID NO: 38 - protein, 6 aa, synthetic construct
  REGION 1..6: note = artificial
RIKSSS

SEQ ID NO: 39 - protein, 6 aa, synthetic construct
  REGION 1..6: note = artificial
RIKSSS

SEQ ID NO: 40 - protein, 6 aa, synthetic construct
  REGION 1..6: note = artificial
RIKSSS

SEQ ID NO: 41 - protein, 6 aa, synthetic construct
  REGION 1..6: note = artificial
RIKSSS

SEQ ID NO: 42 - protein, 6 aa, synthetic construct
  REGION 1..6: note = artificial
RIKSSS

SEQ ID NO: 43 - protein, 6 aa, synthetic construct
  REGION 1..6: note = artificial
RIKSSS

SEQ ID NO: 44 - protein, 6 aa, synthetic construct
  REGION 1..6: note = artificial
RIKSSS

SEQ ID NO: 45 - protein, 6 aa, synthetic construct
  REGION 1..6: note = artificial
RIKSSS

SEQ ID NO: 46 - protein, 6 aa, synthetic construct
  REGION 1..6: note = artificial
RIKSSS

SEQ ID NO: 47 - protein, 6 aa, synthetic construct
  REGION 1..6: note = artificial
KIKDSS

SEQ ID NO: 48 - protein, 6 aa, synthetic construct
  REGION 1..6: note = artificial
KIKDSS

SEQ ID NO: 49 - protein, 6 aa, synthetic construct
  REGION 1..6: note = artificial
KIKDNC

SEQ ID NO: 50 - protein, 6 aa, synthetic construct
  REGION 1..6: note = artificial
KIKDSS

SEQ ID NO: 51 - protein, 6 aa, synthetic construct
  REGION 1..6: note = artificial
KIKDSS

SEQ ID NO: 52 - protein, 6 aa, synthetic construct
  REGION 1..6: note = artificial
KIKDSS

SEQ ID NO: 53 - protein, 6 aa, synthetic construct
  REGION 1..6: note = artificial
NISSNA

SEQ ID NO: 54 - protein, 6 aa, synthetic construct
  REGION 1..6: note = artificial
ELKYNC

SEQ ID NO: 55 - protein, 6 aa, synthetic construct
  REGION 1..6: note = artificial
ILKKST

SEQ ID NO: 56 - protein, 1286 aa, synthetic construct
  REGION 1..1286: note = BoNT/BC
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 57 - protein, 1291 aa, synthetic construct
  REGION 1..1291: note = BoNT/B(0)
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHQLIYVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 58 - protein, 1285 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGSDVEIIPQIERLQLREKALGHYKDIAKRLNNINKTIPSSWSSNIDKYKKIFSEKYNFDKDNTGNFVVNIDKFNSLYSDLTNVMSEVVYSSQYNVKNRTHYFSKHYLPVFANILDDNIYTIINGFNLTTKGFNIENSGQNIERNPALQKLSSESVVDLFTKVCLRLTRNSRDDSTCIQVKNNTLPYVADKDSISQEIFESQIITDETNVENYSDNFSLDESILDAKVPTNPEAVDPLLPNVNMEPLNVPGEEEVFYDDITKDVDYLNSYYYLEAQKLSNNVENITLTTSVEEALGYSNKIYTFLPSLAEKVNKGVQAGLFLNWANEVVEDFTTNIMKKDTLDKISDVSAIIPYIGPALNIGNSALRGNFKQAFATAGVAFLLEGFPEFTIPALGVFTFYSSIQEREKIIKTIENCLEQRVKRWKDSYQWMVSNWLSRITTQFNHISYQMYDSLSYQADAIKAKIDLEYKKYSGSDKENIKSQVENLKNSLDVKISEAMNNINKFIRECSVTYLFKNMLPKVIDELNKFDLKTKTELINLIDSHNIILVGEVDRLKAKVNESFENTIPFNIFSYTNNSLLKDMINEYFNSINDSKILSLQNKKNTLMDTSGYNAEVRVEGNVQLNPIFPFDFKLGSSGDDRGKVIVTQNENIVYNAMYESFSISFWIRINKWVSNLPGYTIIDSVKNNSGWSIGIISNFLVFTLKQNENSEQDINFSYDISKNAAGYNKWFFVTITTNMMGNMMIYINGKLIDTIKVKELTGINFSKTITFQMNKIPNTGLITSDSDNINMWIRDFYIFAKELDDKDINILFNSLQYTNVVKDYWGNDLRYDKEYYMINVNYMNRYMSKKGNGIVFNTRKNNNDFNEGYKIIIKRIIGNTNDTRVRGENVLYFNTTIDNKQYSLGMYKPSRNLGTDLVPLGALDQPMDEIRKYGSFIIQPCNTFDYYASQLFLSSNATTNRIGILSIGSYSFKLGDDYWFNHEYLIPVIKIEHYASLLESTSTHWVFVPASE

SEQ ID NO: 59 - protein, 1280 aa, Clostridium botulinum
MPITINNFNYSDPVDNKNILYLDTHLNTLANEPEKAFRIIGNIWVIPDRFSRDSNPNLNKPPRVTSPKSGYYDPNYLSTDSEKDTFLKEIIKLFKRINSREIGEELIYRLATDIPFPGNNNTPINTFDFDVDFNSVDVKTRQGNNWVKTGSINPSVIITGPRENIIDPETSTFKLTNNTFAAQEGFGALSIISISPRFMLTYSNATNNVGEGRFSKSEFCMDPILILMHELNHAMHNLYGIAIPNDQRISSVTSNIFYSQYNVKLEYAEIYAFGGPTIDLIPKSARKYFEEKALDYYRSIAKRLNSITTANPSSFNKYIGEYKQKLIRKYRFVVESSGEVAVDRNKFAELYKELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDIKTDIFLSKDINEETEVIDYPDNVSVDQVILSKNTSEHGQLDLLYPIIEGESQVLPGENQVFYDNRTQNVDYLNSYYYLESQKLSDNVEDFTFTTSIEEALDNSGKVYTYFPKLADKVNTGVQGGLFLMWANDVVEDFTTNILRKDTLDKISDVSAIIPYIGPALNISNSVRRGNFTEAFAVTGVTILLEAFQEFTIPALGAFVIYSKVQERNEIIKTIDNCLEQRIKRWKDSYEWMIGTWLSRITTQFNNISYQMYDSLNYQADAIKDKIDLEYKKYSGSDKENIKSQVENLKNSLDIKISEAMNNINKFIRECSVTYLFKNMLPKVIDELNKFDLKTKTELINLIDSHNIILVGEVDRLKAKINESFENTIPFNIFSYTNNSLLKDIINEYFNSINDSKILSLQNKKNALVDTSGYNAEVRLEGDVQVNTIYTNDFKLSSSGDKIIVNLNNNILYSAIYENSSVSFWIKISKDLTNSHNEYTIINSIKQNSGWKLCIRNGNIEWILQDINRKYKSLIFDYSESLSHTGYTNKWFFVTITNNIMGYMKLYINGELKQSERIEDLDEVKLDKTIVFGIDENIDENQMLWIRDFNIFSKELSNEDINIVYEGQILRNVIKDYWGNPLKFDTEYYMINYNYIDRYIAPKNNILVLVQYSDISKLYTKNPITIKSAANKNPYSRILNGDDIMFHMLYDSREYMIIRDTDTIYATQGGQCSKNCVYALKLQSNLGNYGIGIFSIKNIVSQNKYCSQIFSSFMKNTMLLADIYKPWRFSFENAYTPVAVTNYETKLLSTSSFWKFISRDPGWVE

SEQ ID NO: 60 - protein, 1291 aa, Clostridium botulinum
MPVTINNFNYNDPIDNDNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVEQKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDTIQAEELYTFGGQDPSIISPSTDKSIYDKVLQNFRGIVDRLNKVLVCISDPNININIYKNKFKDKYKFVEDSEGKYSIDVESFNKLYKSLMFGFTEINIAENYKIKTRASYFSDSLPPVKIKNLLDNEIYTIEEGFNISDKNMGKEYRGQNKAINKQAYEEISKEHLAVYKIQMCKSVKVPGICIDVDNENLFFIADKNSFSDDLSKNERVEYNTQNNYIGNDFPINELILDTDLISKIELPSENTESLTDFNVDVPVYEKQPAIKKVFTDENTIFQYLYSQTFPLNIRDISLTSSFDDALLVSSKVYSFFSMDYIKTANKVVEAGLFAGWVKQIVDDFVIEANKSSTMDKIADISLIVPYIGLALNVGDETAKGNFESAFEIAGSSILLEFIPELLIPVVGVFLLESYIDNKNKIIKTIDNALTKRVEKWIDMYGLIVAQWLSTVNTQFYTIKEGMYKALNYQAQALEEIIKYKYNIYSEEEKSNININFNDINSKLNDGINQAMDNINDFINECSVSYLMKKMIPLAVKKLLDFDNTLKKNLLNYIDENKLYLIGSVEDEKSKVDKYLKTIIPFDLSTYTNNEILIKIFNKYNSEILNNIILNLRYRDNNLIDLSGYGAKVEVYDGVKLNDKNQFKLTSSADSKIRVTQNQNIIFNSMFLDFSVSFWIRIPKYRNDDIQNYIHNEYTIINCMKNNSGWKISIRGNRIIWTLIDINGKTKSVFFEYNIREDISEYINRWFFVTITNNLDNAKIYINGTLESNMDIKDIGEVIVNGEITFKLDGDVDRTQFIWMKYFSIFNTQLNQSNIKEIYKIQSYSEYLKDFWGNPLMYNKEYYMFNAGNKNSYIKLVKDSSVGEILIRSKYNQNSNYINYRNLYIGEKFIIRRKSNSQSINDDIVRKEDYIHLDFVNSNEEWRVYAYKNFKEQEQKLFLSIIYDSNEFYKTIQIKEYDEQPTYSCQLLFKKDEESTDDIGLIGIHRFYESGVLRKKYKDYFCISKWYLKEVKRKPYKSNLGCNWQFIPKDEGWTE

SEQ ID NO: 61 - protein, 1292 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGGEERKEGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNGKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDDEIYTIEEGFNISDKNMGKEYRGQNKAINKQAYEEISKEHLAVYKIQMCKSVRAPGICIDVDNEDLFFIADKNSFSDDLSKNERIEYNTQSNYIENDFSINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVDDFVIEANKSNTMDKIADISLIVPYIGLALNVGNETAKGNFENAFEIAGSSILLEFIPELLIPVVGAFLLESYIDNKNKIIKTIDNALTKRDEKWIDMYGLIVAQWLSTVNTQFYTIKEGMYKALNYQAQALEEIIKYKYNIYSEKEKSNISIDFNDINSKLNEGINQAIDNINDFINECSVSYLMKKMIPLAVEKLLDFDNTLKKNLLNYIDENKLYLIGSAEYEKSKVDKHLKTIMTFDLSMYTNNTILIKMVNKYNSEILNNIILNLRYRDNNLIDLSGYGANVEVYDGVELNDKNQFKLTSSTNSEIRVTQNQNIIVNSMFLDFSVSFWIRIPKYKNDGIQNYIHNEYTIINCMKNNSGWKISIRGNRIIWTLIDINGKIKSVFFEYSIRKDVSEYINRWFFVTITNNLDNAKIYINGKLESNMDIRDIREVIANGEIIFKLDGDIDRTQFIWMKYFSIFNTELSQSNIEEIYKIQSYSEYLKDFWGNPLMYNKEYYMFNAGSKNSYIKLKKDSSVGEILTRSKYNQNSQYINYRDLYIGEKFIIKRKSNSQSINDDIVRKEDYIYLDFFNLNQEWRVYAYKDFKGQKEQKLFLANIHDSNEFYKTIQIKEYDEQPTYSCQLLFKKDEESTDEIGLIGIHRFYESGFVFQEYKYYFCISKWYLKEVKKKPYNPDLGCNWQFIPKDEGWTE

SEQ ID NO: 62 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTKNIYIENYFSINELILDTDLISGIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIIDDFVIEANKSSTMDKIADISLIVPYIGLALNVGNETAKGNFENAFEIAGASILLEFIPELLIPVVGAFLLESYIDNKNKIIKTIDNALTKRVEKWIDMYGLIVAQWLSTVNTQFYTIKEGMYKALNYQAQALEEIIKYKYNIYSEKEKLNINIDFNDINSKLNEGINQAIDNINNFINECSVSYLMKKMIPLAIEKLLDFDNALKKNLLNYIDENKLYLIGSVEEEKSKVDKFFKTIIPFDLSMYTNNTILIEMVNKYNSEILNNIILNLRYRDNNLIDSSGYGAKVEVYNGVELNDKNQFKLTSSANSKIKVTQNQNITFNSMFLDFSVSFWIRIPKYKNDGIQNYIHNEYTIINCMKNNSGWKISIRGNRIIWTLTDINGKTKSVFFEYSIREDISDYINRWFFVTITNNLDNAKIYINGKLESNIDIRDIREVIVNGEIIFKLDGEIDRTQFIWMKYFSIFNTELSQSNVKEIYKIQSYSKYLKDFWGNPLMYNKEYYMFNAGNKNSYIKLVKDSSVGEILTRSKYNQNSNYINYRNLYIGEKFIIRRKSSSQSISDDIVRKEDYIYLDFFNSNREWRVYAYKNFKGQEEKLFLANIYDSNEFYKTIQIKEYDEQPTYSCQLLFKKDEESTDEIGLIGIHNFYESGILFKDYKDYFCISKWYLKEVKKKPYSSNLGCNWQFIPKDEGWTE

SEQ ID NO: 63 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKNMEKEYRGQNKAINKQAYEEISKEHLAVYKIQMCKSVRAPGICIDVDNEDLFFIADKNSFSDDLSKNERIEYDTQSNYIENRSSIDELILDTNLISKIELPSENTESLTDFNVDVPVYEKQPAIKKFFTDENTIFQYLYSQTFPLDIRDISLTSSFDDALLFSNKVYSFFSMDYIKTANKVVEAGLFAGWVKQIVDDFVIEANKSNTMDKLADISLIVPYIGLALNVGNETAKGNFENAFEIAGASILLEFIPELLIPVVGAFLLESYIDNKNKIIKTIDNALTKRDEKWRDMYGLIVAQWLSTVNTQFYTIKEGMYKALNYQAQALEEIIKYKYNIYSEKEKSNINIDFNDINSKLNEGINQAIDNINNFINECSVSYLMKKMIPLAVEKLLDFDNTLKKNLLNYIDENKLYLIGSAEYEKSKVDKHLKTIIPFDLSMYTNNTILIEIFKKYNSEILNNIILNLRYRDNNLIDLSGYGANVEVYDGVELNDKNQFKLTSSTNSEIRVTQNQNIIFNSMFLDFSVSFWIRIPKYKNDGIQNYIHNEYTIINCIKNNSGWKISIRGNRIIWTLTDINGKTKSVFFEYSIREDISDYINRWFFVTITNNSDNAKIYINGKLESNIDIKDIGEVIANGEIIFKLDGDIDRTQFIWMKYFSIFNTELSQSNIKEIYKIQSYSEYLKDFWGNPLMYNKEYYMFNAGNKNSYIKLKKDSPVGEILTRSKYNQNSNYINYRNLYIGEKFIIRRKSNSQSINDDIVRKEDYIYLDFFNLNQEWRVYALKNFKKKEEKLFLAPISDSDEFYNTIQIKEYDEQPTYSCQLLFKKDEESTDEIGLIGIHRFYESGIVFKDYKYYFCISKWYLKEVKRKPYNPNLGCNWQFIPKDEGWIE

SEQ ID NO: 64 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKNMEKEYRGQNKAINKQAYEEISKEHLAVYKIQMCKSVRAPGICIDVDNEDLFFIADKNSFSDDLSKNERIEYDTQSNYIENRSSIDELILDTNLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVDDFVIEANKSSTMDKIADISLIVPYIGLALNVGNETAKGNFENAFEIAGASILLEFIPELLIPVVGAFLLESYIDNKNKIIKTIDNALTKRDEKWIDMYGLIVAQWLSTVNTQFYTIKEGMYKALNYQAQALEEIIKYKYNIYSEKEKSNINIDFNDINSKLNEGINQAVDNINNFINECSVSYLMKKMIPLAVEKLLDFDNTLKKNLLNYIDENKLYLIGSAEYEKSKVDKHLKTIIPFDLSMYTNNTILIEIFNKYNSEILNNIILNLRYRDNNLIDLSGYGANVEVYDGVELNDKNQFKLTSSTNSEIRVTQNQNIIFNSMFLDFSVSFWIRIPKYKNDGIQNYIHNEYTIINCIKNNSGWKISIRGNRIIWTLTDINGKTKSVFFEYSIREDISDYINRWFFVTITNNSDNAKIYINGKLESNIDIKDIGEVIANGEIIFKLDGDIDRTQFIWMKYFSIFNTELSQSNIKEIYKIQSYSEYLKDFWGNPLMYNKEYYMFNAGNKNSYIKLKKDSSVGEILTRSKYNQNSNYINYRNLYIGEKFIIRRKSNSQSINDDIVRKEDYIYLDFFNSNREWRVYAYKDFKEEEKKLFLANIYDSNEFYKTIQIKEYDEQPTYSCQLLFKKDEESTDEIGLIGIHRFYESGIVLKDYKNYFCISKWYLKEVKRKPYNPNLGCNWQFIPKDEGWIE

SEQ ID NO: 65 - protein, 1291 aa, Clostridium botulinum
MPVTINNFNYNDPIDNDNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPRIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKNMEKEYRGQNKAINKQAYEEISKEHLAVYKIQMCKSVRAPGICIDVDNEDLFFIADKNSFSDDLSKNERIEYDTQSNYIENRSSIDELILDTNLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVDDFVIEANKSSTMDKIADISLIVPYIGLALNVGNETAKGNFENAFEIAGASILLEFIPELLIPVVGAFLLESYIDNKNKIIKTIDNALTKRDEKWIDMYGLIVAQWLSTVNTQFYTIKEGMYKALNYQAQALEEIIKYKYNIYSEKEKSNINIDFNDINSKLNEGINQAIDNINNFINECSVSYLMKKMIPLAVEKLLDFDNTLKKNLLNYIDENKLYLIGSAEYEKSKVDKHLKTIIPFDLSMYTNNTILIEIFNKYNSEILNNIILNLRYRDNNLIDLSGYGAKVEVYNGVELNDKNQFKLTSSANSKIRVTQNQDIIFNSMFLDFSVSFWIRIPKYKNDGIQNYIHNEYTIINCIKNNSGWKISIRGNKIIWTLTDINGKTKSVFFEYSIRKDVSEYINRWFFVTITNNSDNAKIYINGKLESNIDIKDIGEVIANGEIIFKLDGDIDRTQFIWMKYFSIFNTELSQSNIKEIYKIQSYSEYLKDFWGNPLMYNKEYYMFNAGNKNSYIKLKKDSSVGEILTRSKYNQNSNYINYRNLYIGEKFIIRRKSNSQSINDDIVRKEDYIYLDFFNLNQEWRVYAYKDFKKKEEKLFLANIYDSNEFYNTIQIKEYDEQPTYSCQLLFKKDEESTDEIGLIGIHRFYESGIVFKDYKDYFCISKWYLKEVKRKPYNPNLGCNWQFIPKDEGWIE

SEQ ID NO: 66 - protein, 1291 aa, Clostridium botulinum
MPVTINNFNYNDPIDNNNIIMMEPPFARGMGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVNDLPIVPNEKKFFMQSTDAIQAEELYTFGGQDPSIISPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKNMEKEYRGQNKAINKQAYEEISKEHLAVYKIQMCKSVKAPGICIDVDNEDLFFIADKNSFSDDLSKNERIAYNTQNNYIENDFSINELILDTDLISKIELPSENTESLTDFNVYVPVYKKQPAIKKIFTDENTIFQYLYSQTFPLDIRDISLTSSFDDALLFSNKVYSFFSMDYIKTANKVVEAGLFAGWVKQIVDDFVIEANKSSTMDKIADISLIVPYIGLALNVGNETAKGNFENAFEIAGASILLEFIPELLIPVVGAFLLESYIDNKNKIIETINSALTKRDEKWIDMYGLIVAQWLSTVNTQFYTIKEGMYKALNYQAQALEEIIKYKYNIYSEKERSNINIDFNDVNSKLNEGINQAIDNINNFINECSVSYLMKKMIPLAVEKLLDFDNTLRKNLLNYIDENKLYLIGSAEYEKSKVDKYLKTSIPFDLSTYTNNTILIEIFNKYNSDILNNIILNLRYRDNKLIDLSGYGAKVEVYDGVKLNDKNQFKLTSSANSKIRVIQNQNIIFNSMFLDFSVSFWIRIPKYKNDGIQNYIHNEYTIINCMKNNSGWKISIRGNMIIWTLIDINGKIKSVFFEYSIKEDISEYINRWFFVTITNNSDNAKIYINGKLESHIDIRDIREVIANDEIIFKLDGNIDRTQFIWMKYFSIFNTELSQSNIEEIYKIQSYSEYLKDFWGNPLMYNKEYYMFNAGNKNSYIKLKKDSSVGEILTRSKYNQNSKYINYRDLYIGEKFIIRRKSNSQSINDDIVRKEDYIYLDFFNLNQEWRVYMYKYFKKEEEKLFLAPISDSDEFYNTIQIKEYDEQPTYSCQLLFKKDEESTDEIGLIGIHRFYESGIVFKEYKDYFCISKWYLKEVKRKPYNSKLGCNWQFIPKDEGWTE

SEQ ID NO: 67 - protein, 1296 aa, Clostridium botulinum
MPLVNQQINYYDPVNGVDIAYIKIPNAGKMQPVKAFKIHNKVWVIPERDIFTNPEEVDLNPPPEAKQVPISYYDSAYLSTDNEKDNYLKGVIKLFERIYSTDLGRMLLISIVRGIPFWGGGKIDTELKVIDTNCINIIQLDDSYRSEELNLAIIGPSANIIESQCSSFRDDVLNLTRNGYGSTQYIRFSPDFTVGFEESLEVDTNPLLGAGKFAQDPAVALAHELIHAEHRLYGIAINTNRVFKVNTNAYYEMAGLEVSLEELITFGGNDAKFIDSLQKKEFSLYYYNKFKDIASTLNKAKSIVGTTASLQYMKNVFKEKYLLSEDATGKFLVDRLKFDELYKLLTEIYTEDNFVKFFKVLNRKTYLNFDKAVFKINIVPDVNYTIHDGFNLRNTNLAANFNGQNIEINNKNFDKLKNFTGLFEFYKLLCVRGIITSKTKSLDEGYNKALNELCIKVNNWDLFFSPSEDNFTNDLDKVEEITSDTNIEAAEENISLDLIQQYYLNFNFDNEPENTSIENLSSDIIGQLEPMPNIERFPNGKKYELNKYTMFHYLRAQEFKHSNSRIILTNSAKEALLKPNIVYTFFSSKYIKAINKAVEAVTFVNWIENLVYDFTDETNEVSTMDKIADITIVIPYIGPALNIGNMIYKGEFVEAIIFSGAVILLEIVPEIALPVLGTFALVSYVSNKVLTVQTIDNALSKRNEKWDEVYKYIVTNWLAIVNTQINLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLIGQVNRLKDKVNNTLSADIPFQLSKYVDNKKLLSTFTEYIKNITNASILSIVYKDDDLIDLSRYGAEIYNGDKVYYNSIDKNQIRLINLESSTIEVILKKAIVYNSMYENFSTSFWIRIPKYFNSISLNNEYTIINCMENNSGWKVSLNYGEIIWTFQDTQEIKQRVVFKYSQMINISDYINRWIFVTITNNRITKSKIYINGRLIDQKPISNLGNIHASNKIMFKLDGCRDPHRYIVIKYFNLFDKELSEKEIKDLYDNQSNSGILKDFWGDYLQYDKSYYMLNLYDPNKYVDVNNVGIRGYMYLKGPRDNVMTTNIYLNSSLYMGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWRERPL

SEQ ID NO: 68 - protein, 1296 aa, Clostridium botulinum
MPFVNKQFNYKDPVNGVDIAYIKIPNAGQMQPVKAFKIHNKIWVIPERDIFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIINFECKSFGHDVLNLTRNGYGSTQYIRFSPDFTFGFEESLEVDTNPLLGAGKFAIDPAVTLAHELIHAGHRLYGIAINPNRVFKVNTNAYYEMSGLEVSFEELRTFGGHDAKFIDSLQENEFRLYYYNKFKEVASILNKAKSIIGTTASLQYMKNVFKEKYLLSEDTSGKFSVDKLRFDKLYKMLTEIYTEDNFVKFFKVLNRKTYLNFDKAVFKMNIVPEVNYTIYDGFNLRNTNLAANFNGQNTEINNMNFTKLKNFTGLFEFYKLLCVRGIITSKTKSLDEGYNKALNDLCIKVNNWDLFFSPSEDNFTNDLNKGEEITSDTNIEAAEENISSDLIQQYYLTFNFDNEPENISIENLSSDIIGQLELMPNIERFPNGKKYELDKYTMFHYLRAQEFEYGNSRIVLINSVNEALLNPSSVYTFFSSDYVKKANEATEAAMFLGWVEQLVYDFTDETSEVSTMDKIADITIIVPYIGPALNIGNMVYKKKFEEALIFSGAVILLEFVPEIVLPILGTFALVSYTSNKVLTVRTIDNALSKRNEKWEEVYKYIVTNWLAKVNTQINLIRKKMKEALENQAEATKAIINYQYNQYTEEEKNNINFNIGDLSSKLNDSINKAMININKFLDQCSVSYLMNSMIPQGVKQLKDFDTSLRDSLLKYIYDNRGTLIGQVDRLKDKVNNTLSTDIPFQLSKYADNQRLLSTFTEYIKNIINTSILNLRYESNHLIDLSRYASKINIGSRVNFDPIDKNQIQLFNLESSKIEVILKNAIVYNSMYENFSTSFWIKIPKYFSKINLNNEYTIINCIENNSGWKVSLNYGEIIWTLQDNEQNIQRVVFKYSQMVNISDYINRWIFVTITNNRLTKSKIYINGRLIDQKPISNLGNIHASNKIMFKLDGCRDPHRYILIKYFNLFDKELNEKEIKDLYDNQSNSGILKDFWGDYLQYDKPYYMLNLYDPNKYIDVNNIGIRGYMYLKGPRGSVTTTNIYLNSMLYMGTKFIIKKHASGNKDNIVRNNDRVYINVLVKNKEYRLATNASQAGGEKILSAVEIPDVGNLSQVVVMKSKNDQGIRNKCKMNLQDNNGNDIGFIGFHQFNNIAKLVASNWYNRQIGKTSVTLGCSWELIPVDYGWGESSL

SEQ ID NO: 69 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFAKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLSAQEFEHGKSRIDLTNSVNEALLNPSHVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLIGQVDRLKDKVNNTLSTDIPFQLSKYVDNQRLLSTFTEYIKNIINTSILSLRYENNHLIDLSRYASKINIGSRVNFDPIDKNQIQLFNLESSKIEVILKNAIVYNSMYENFSTSFWIKIPKYFSEISLNNEYTIINCIENNSGWKVSLNYGEIIWTLQDNKQNIQRVVFKYSQMVAISDYINRWIFITITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNVGIRGYMYLKGSRSTLLTTNIYLNSGLYMGTKFIIKKYASGNKDNIVRNNDRVYINVVVNNKEYRLATNASQAGVEKILSALEIPDIGNLSQVVVMKSKNDQGIRNKCKMNLQDNNGNDIGFIGFHKFNDIYKLVASNWYNRQIEISSRTFGCSWEFIPVDDGWGEKPL

SEQ ID NO: 70 - protein, 1296 aa, Clostridium botulinum
MLFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTELGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGEHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPEVNYTIYDGFNLRNTNLAANFNGQNTEINNMNFTKLKNFTGLFEFYKLLCVRGIITSKTKSLDEGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIVLTNSVNEALLNPSSVYTFFSSDYVRKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWGEVYKYIVTNWLAKVNTQIDLIRKKMKEALENQAEATKAIINYQYNQYTEEEKNNINFNIGDLSSKLNDSINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASEINIGSKVNFDPIDKNQIQLFNLESSKIEIILKNAIVYNSMYENFSTSFWIKIPKYFSKINLNNEYTIINCIENNSGWKVSLNYGEIIWTLQDNKQNIQRVVFKYSQMVAISDYINRWIFITITNNRLNNSKIYINGRLIDQKPISNLGNIHASNNIMFKLDGCRDPHRYIWIKYFNLFDKELNEKEIKDLYDNQSNSGILKDFWGNYLQYDKPYYMLNLYDPNKYVDVNNVGIRGYMYLKGPRGSIVTTNIYLNSSLYMGTKFIIKKYASGNKDNIVRNNDRVYINVVVKNKEYRLATNASQAGVEKILSVLEIPDVGNLSQVVVMKSKNDQGIRNKCKMNLQDNNGNDIGFIGFHQFNNIDKLVASNWYNRQIERSSRTFGCSWEFIPVDDGWGESPL

SEQ ID NO: 71 - protein, 1292 aa, Clostridium botulinum
MPFVNKPFNYRDPGNGVDIAYIKIPNAGQMQPVKAFKIHEGVWVIPERDTFTNPEEGDLNPPPEAKQVPVSYYDSTYLSTDNEKDNYLKGVIKLFDRIYSTGLGRMLLSFIVKGIPFWGGSTIDTELKVIDTNCINVIEPGGSYRSEELNLVITGPSADIIQFECKSFGHDVFNLTRNGYGSTQYIRFSPDFTFGFEESLEVDTNPLLGAGTFATDPAVTLAHELIHAAHRLYGIAINPNRVLKVKTNAYYEMSGLEVSFEELRTFGGNDTNFIDSLWQKKFSRDAYDNLQNIARILNEAKTIVGTTTPLQYMKNIFIRKYFLSEDASGKISVNKAAFKEFYRVLTRGFTELEFVNPFKVINRKTYLNFDKAVFRINIVPDENYTINEGFNLEGANSNGQNTEINSRNFTRLKNFTGLFEFYKLLCVRGIIPFKTKSLDEGYNKALNYLCIKVNNWDLFFSPSEDNFTNDLDKVEEITADTNIEAAEENISSDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAVIFLSWAEELVYDFTDETNEVTTMDKIADITIIVPYIGPALNIGNMVSKGEFVEAILFTGVVALLEFIPEYSLPVFGTFAIVSYIANKVLTVQTINNALSKRNEKWDEVYKYTVTNWLAKVNTQIDLIREKMKKALENQAEATRAIINYQYNQYTEEEKNNINFNIDDLSSKLNRSINRAMININKFLDQCSVSYLMNSMIPYAVKRLKDFDASVRDVLLKYIYDNRGTLILQVDRLKDEVNNTLSADIPFQLSKYVNDKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWMFVTITNNRLTKSKIYINGRLIDQKPISNLGNIHASNKIMFKLDGCRDPRRYIMIKYFNLFDKELNEKEIKDLYDSQSNPGILKDFWGNYLQYDKPYYMLNLFDPNKYVDVNNIGIRGYMYLKGPRGSVMTTNIYLNSTLYMGTKFIIKKYASGNEDNIVRNNDRVYINVVVKNKEYRLATNASQAGVEKILSALEIPDVGNLSQVVVMKSKDDQGIRNKCKMNLQDNNGNDIGFVGFHLYDNIAKLVASNWYNRQVGKASRTFGCSWEFIPVDDGWGESSL

SEQ ID NO: 72 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDEGYNKALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 73 - protein, 1297 aa, Clostridium botulinum
MPFVNKQFNYKDTVNGIDIAYIKIPNAGQMQPVKAFKIHNKIWVIPERDTFTNPKEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHNAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPDENYTIKDGFNLKNTNLAANFNGQNTEINSRNFTKLKNFTGLFEFYKLLCVRGIIPFKTKSLDEGYNKALNDLCIKVNNWDLFFSPSEDNFTNDLDKVEEITSDTNIEAAEENISLDLIQQYYLTFDFDNEPENISIENLSSDIIGQLEPMPNIERFPNGKKYELDKYTMFHYLRAQEFEHSKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLVRKKMKEALENQAEATKAIINYQYNQYTEEEKNNINFNIDDLSSKLNESINSAMTNINKFLDQCSVSYLMNSMIPYAVKRLKDFDASVREVLLKYIYDNRGTLILQVDRLKDKVNNTLSADIPFQLSKYVDNKKLLSTFTEYIKNITNTSILSIVVDKDGRLIDLSRYGAEIYNGDKVSYNSIDKNQIKLINLESSAIEVILKNAIVYNSMYENFSTSFWIKIPKYFSKINLNNEYTIINCIENNSGWKVSLNYGEIIWTLQDNQQNIQRVVFKYSQMVNISDYINRWIFVTITNNRLDKSKIYINGRLIDQKPISNLGNIHASNNIMFKLDGCRDPRRYIVIKYFNLFDKELNEKEIKDLYDNQSNSGILKDFWGDYLQYDKPYYMLNLYDPNKYVDVNNIGIRGYMYLKGPRGSVVTTNIYLNSTLYMGTKFIIKKYASGNKDNIVRNNDRVYINVVVKNKEYRLATNALQAGVEKILSALEIPDVGNLSQVVVMKSKNDQGIRNKCKMNLQDNNGNDIGLIGFHQFNNIAKLVASNWYNRQVGKASRTFGCSWEFIPVDDGWGESSQ

SEQ ID NO: 74 - protein, 1288 aa, Clostridium botulinum
MPVVINSFNYDDPVNDNTIIYIRPPYYETSNTYFKAFQIMDNVWIIPERYRLGIDPSLFNPPVSLKAGSDGYFDPNYLSTNTEKNKYLQIMIKLFKRINSKPAGQILLEEIKNAIPYLGNSYTQEEQFTTNNRTVSFNVKLANGNIVQQMANLIIWGPGPDLTTNKTGGIIYSPYQSMEATPYKDGFGSIMTVEFSPEYATAFNDISIASHSPSLFIKDPALILMHELIHVLHGLYGTYITEYKITPNVVQSYMKVTKPITSAEFLTFGGRDRNIVPQSIQSQLYNKVLSDYKRIASRLNKVNTATALINIDEFKNLYEWKYQFAKDSNGVYSVDLNKFEQLYKKIYSFTEFNLAYEFKIKTRLGYLAENFGPFYLPNLLDDSIYTEVDGFNIGALSINYQGQNIGSDINSIKKLQGQGVVSRVVRLCSNSNTKNSLCITVNNRDLFFIASQESYGENTINTYKEIDDTTTLDPSFEDILDKVILNFNEQVIPQMPNRNVSTDIQKDNYIPKYDYNRTDIIDSYEVGRNYNTFFYLNAQKFSPNESNITLTSSFDTGLLEGSKVYTFFSSDFINNINKPVQALLFIEWVKQVIRDFTTEATKTSTVDKLKDISLVVPYIGLALNIGDEIYKQHFAEAVELVGAGLLLEFSPEFLIPTLLIFTIKGYLTGSIRDKDKIIKTLDNALNVRDQKWKELYRWVVSKWLTTINTQFNKRKEQMYKALKNQATAIKKIIENKYNNYTTDEKSKIDSSYNINEIERTLNEKINLAMKNIEQFITESSIAYLINIINNETIQKLKSYDDLVRRYLLGYIRNHSSILGNSVEELNSKVNNHLDNGIPFELSSYTNDSLLIRYFNKNYGELKYNCILNIKYEMDRDKLVDSSGYRSRINIGTGVKFSEIDKNQVQLSNLESSKIEVILNNGVIYNSMYENFSTSFWIRIPKYFRNINNEYKIISCMQNNSGWEVSLNFSNMNSKIIWTLQDTEGIKKTVVFQYTQNINISDYINRWIFVTITNNRLSNSKIYINGRLINEESISDLGNIHASNNIMFKLDGCRDPHRYIWIKYFNLFDKELNKKEIKDLYDNQSNSGILKDFWGDYLQYDKPYYMLNLYDPNKYLDVNNVGIRGYMYLKGPRGRIVTTNIYLNSTLYMGTKFIIKKYASGNKDNIVRNNDRVYINVVVKNKEYRLATNASQAGVEKILSAVEIPDVGNLSQVVVMKSENDQGIRNKCKMNLQDNNGNDIGFIGFHQFNNIAKLVASNWYNRQIGKASRTFGCSWEFIPVDDGWGESSL

SEQ ID NO: 75 - protein, 1251 aa, Clostridium botulinum
MPKINSFNYNDPVNDNTILYIKPGGCQQFYKSFNIMKNIWIIPERNVIGTIPQNFLPPTSLKNGDSSYYDPNYLQNDQEKDRFLKIVTKVFNRINDNLSGRILLEELSKANPYLGNDNTRDDDFIINDGSAVPIQFSNGSQSILLPTVIIMGAEPDLFETNSSNVSLINNYSPSNHGFGSIAIVTFSPEYSFRFNDNSMNEFIQDPALTLIHELIHSLHGLYGAKGITTKYTITQQQNPLITNIRGINIEEFLTFGGNNLNIITSSQLNDIYTNLLDDYKKIASKLSKVQVSNPQLNPYKDVFQEKYGLDKNASGIYSVNINKFNDIFKKLYSFTEFDLATKFQVKCRETYIGQYKYFKLSNLLNDSIYNISEGYNINTLNVNFRGQNPNLNPRIITPITDRGLVKKIIRFCKNIVSVKGIRKSICIEVNNGDLFFVASEKSYNNDSINIPKEIDDTVTLNNNYENDLDQVILNFNSESAPGLSDKKLNISIQDDVYIPKYDSNGTSDIEQYDVSELNVFFYLDAQKVPEGENNVNLTSSIDTALLEQSKIYTFFSSEFINNVNKPVQAALFVGWIQQVLVDFTTEATQKSTVDKIADISIVVPYIGLALNIGNESQKGNFKDALELLGAGILLEFVPELLIPTILVFTIKSFLGSSDNKNKVIKAINNALKERDEKWKEVYSFIVSNWITKINTQFNKRKEQMYQALQNQVNALKTIIESKYNSYTLEEKNELTNKYDIEQIENELNQKVSIAMNNIDRFLTESSISYLMKLINEVKINKLREYDENVKTYLLDYITKHGSILGESQQELNSMIIDTLNNSIPFKLSSYTDDKILISYFNKFFKTIKSSSVLSMRYKNDKYIDTSGYDSNININGDVFIYPTNKNQFGIYNSKLSEVNISQNDYIIYDNKYKNFSISFWVRIPNYNNKIVNVNNEYTIINCMRDNNSGWKISLNHNEIIWTLQDNAGINQKLVFKYGNANGISDYINKWIFVTITNDRLGYSKLYINGHLIDQKSILNLGNIHVSDNILFKIVNCSYTRYIGMRYFNIFDKELDETEIQTLYNNEPNANVLKDFWGNYLLYNKEYYLLNMLKPSKTISHNRDLTFSIYNNRNIVNGLYRLYSGIKVKIQKINDSDTRDNIVRDNDQVYVNYINGNVYYSLYADTNATNKEKTIKSSTSGNRFNQVVVMNSVRNNCTMNFKNNNGHDIGLLGFKSNALVASTWYYTNMRDHTNSNGCFWSFIPEENGWQEH

SEQ ID NO: 76 - protein, 1254 aa, Clostridium botulinum
MLYMPKINSFNYNDPVNDRTILYIKPGGCQQFYKSFNIMKNIWIIPERNVIGTIPQDFQPPTSLKNGDSSYYDPNYLQSNEEKDRFLKIVTKIFNRINDNLSGGILLEELSKANPYLGNDNTPDGDFIINDASAVPIQFSNGSQSILLPNVIIMGAEPDLFETNSSNISLINNYRPSNHGFGSIAIVTFSPEYSFRFNDNSMNEFIQDPALTLMHELIHSLHGLYGAKGITTKYTITQQQNSLITNIRGINIEEFLTFGGNDLNIITSSQFNDIYTNLLDDYKKIASKLSQVRVSNPQLNPYKDVFQEKYGLDKDASGIYSVNINKFNDIFKKLYSFTEFDLATKFQVKCRETYIGQYKYFQLSNLLNDSIYNISEGYNINNLKVNFRGQNANLNPRIITPITGRGLVKKIIRFCKNIVSVKGIRKSICIEVNNGELFFVASENSYNDDNINTPKEIDDTVTSNNNYENDLDQVILNFNSESAPGLSDEKLNLTIQDDAYIPKYDSNGTSDIEQHDVNELNVFFYLDAQKVPEGENNVNLTSSIDTALLEQPKIYTFFSSEFINNVNKPVQAVLFVSWIQQVLVDFTTEATQKSTVDKIADISIVVPYIGLALNIGNEAQKGNFKDALELLGAGILLEFVPELLIPTILVFTIKSFLGSSDNKNKIIKAINNALKERDEKWKEVYSFIVSNWITKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSTLGESQQELNSMVINTLNNSIPFKLSSYTDDKILISYFNKFFKRIKSSSVLNMRYKNDKYVDTSGYDSNININGEIFIYPTNKNQFSIFNSKPSEVNISQNDYIIYDNKYKNFSISFWVRIPNYDNKIVNVNNEYTIINCMRDNNSGWKVSLNHNEIIWTLQDNAGINQKLAFNYGNSNGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNSIISHRRDLTFSFYNHRYIVNGLYRLYSGIKVKIQRVNDSSTNDQFVRKNDQVYINYIYNNLSYSLYADTNIKDKEKTIKSSLSGNIFNQVVVMNSVGNNCTMNFKNNNGNNIGLLGFKDNTLVASTWYYTHMRDNTNSNGCFWNFISEEHGWQEK

SEQ ID NO: 77 - protein, 1252 aa, Clostridium botulinum
MPKINSFNYNDPVNDKTILYIKPGGCQQFYKSFNIMKNIWIIPERNVIGTIPQDFLPPTSLKNGDSSYYDPNYLQSNEEKDRFLKIVTKIFNRINDNLSGGILLEELSKANPYLGNDNTPNNQFHIGDASAVEIKFSNGSQSILLPTVIIMGAEPDLFETNSSNISLKNNYMPSNHGFGSIAIVTFSPEYSFRFNDNSMNEFIQDPALTLMHELIHSLHGLYGAKGITTKYTITQQQNPLITNIRGTNIEEFLTFGGTDLNIITNAQSNDIYTNLLDDYKKIASKLSQVQVSNPQLNPYKDVFQEKYGLDKDANGIYSVNINKFNDIFKKLYSFTEFDLATKFQVKCRKTYIGHHKYFRLSDLLNDSIYNISDGYNINTLKVNFRGQNTNLNTRIITPITGRGVVRKIIRFCTNIFSPKGIRKSICIEVNNGELFFVASENSYNDDNINTSKEIDDTVTSNNNYENDLDQVILNFNSESAPGLSDEKLNLTIQDDAYIPKYDSNGTSDIEQYDVSELNVFFYLDAQKVPEGENNVDFTSSIDTALLEQPKIYTFFSSKFISNLNKTMQAALFVSWIQQVLVDFTTEATQKSTVDKIADISIVVPYIGLALNIGNEAQKGNFKDALELLGAGILLEFEPELLIPIILVFTIKSFLGSSDNKNKVIKAINNALKERDENWKEVYSFIVSNWMTKINTQFNKRKEQMYQALQNQVNAIKTIIESKYNSYTLEEKNELKNKYDIEQIENELNQTVSIAMNNIEIFLTESSISYLMKLINEVKINKLKEYDENVKTYLLDYIIKHGSILGESQQELNSMVIDTLNNSIPFKLSSYTDDKILISYFNKFFKTIKSSSVLNMRYKNDKYIDTSGYDSNINIKGDVFIYPTNKNQFGIYNNKLSEVNISQNDYIIYDNKYKNFSISFWVRIPNYDNKIVNVNNEYTIINCMRDNNSGWKVSLNHNEIIWTLQDNAGINQKLVFKYGNANGISDYINKWIFVTITNDRLGDSKLYINGNLIDKKSILNLGNIHVSDNILFKIVNCSYTRYIGMRYFNIFDKELDKTEIETLYNNEPNTNILKDFWGNYLLYDKEYYLLNVLKPNNVIDSNRDSTFSIHNIRSTIVLANRLYSGIKVKIQRVNNSSTNDNLVRKNDQVYINFVASKTHLFPLYADTNTTNKEKTIKSSSSGNRFNQVVVMNSVGNNCTMNFKNNNGNNIGMLGFKDNTLVASTWYYTHMRDNTNSNGCFWNFISEEHGWQEK

SEQ ID NO: 78 - protein, 1252 aa, Clostridium botulinum
MPKINSFNYNDPVNDKTILYIKPGGCQQFYKSFNIMKNIWIIPERNVIGTIPQDFLPPTSLKNGDSSYYDPNYLQSNEEKDRFLKIVTKIFNRINDNLSGGILLEELSKANPYLGNDNTPNNQFHIGDASAVEIKFSNGSQSILLPTVIIMGAEPDLFETNSSNISLKNNYMPSNHGFGSIAIVTFSPEYSFRFNDNSMNEFIQDPALTLMHELIHSLHGLYGAKGITTKYTITQQQNPLITNIRGTNIEEFLTFGGTDLNIITNAQSNDIYTNLLADYKKIASKLSQVQVSNPQLNPYKDIFQEKYGLDKNASGIYSVNINKFDDIFKKLYSFTEFDLATKFQVKCRQTYIGQYKYFKLSNLLNNSIYNISEGYNINTLKVNFRGQNTNLNPRIITQLTGRGLVKKIIRFCKNIVFSKGITKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKTIIESKYNSYTLEEKNELTNKYNIEQIENELNQKVSIAMNNIEIFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDKKSILNLGNIHVSDNILFKIVNCSYTRYIGMRYFNIFDKELDKTEIETLYNNEPNTNILKDFWGNYLLYDKEYYLLNVLKPNNVIDSNRDSTFSIHNIRSTIVLANKLYLGIKVKIQRVNNSSTNDNLVRKNDQVYINFVPIKTHLFPLYADTNTTNKEKTIKSSSSGNRFNQVVVMNSVGNNCTMNFKNNNGNNIGMLGFKDNTLVASTWYYTHMRDNTNSNGCFWNFISEEHGWQEK

SEQ ID NO: 79 - protein, 1252 aa, Clostridium botulinum
MPKINSFNYNDPVNDKTILYIKPGGCQQFYKSFNIMKNIWIIPERNVIGTIPQDFLPPTSLKNGDSSYYDPNYLQSNEEKDRFLKIVTKIFNRINDNLSGRILLEELSKANPYLGNDNTPDNQFHIGDASAVEIKFSNGNQSILLPNVIIMGAEPDLFETNSSNISLRNNYMPSNHGFGSIAIVTFSPEYSFRFNDNSMNEFIQDPALTLMHELIHSLHGLYGAKRITTKYTITQQQNPLITNIRGTNIEEFLTFGGTDLNIITSAQYNDIYTNLLADYKKIASKLSKVQVSNPQLNPYKDIFQEKYGLDKNASGIYSVNINKFDDIFKKLYSFTEFDLATKFQVKCRQTYIGQYKYFKLSNLLNNSIYNISEGYNINTLKVNFRGQNTNLNPRIITQLTGRGLVKKIIRFCKNIVFSKGITKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 80 - protein, 1252 aa, Clostridium botulinum
MPKINSFNYNDPVNDKTILYIKPGGCQQFYKSFNIMKNIWIIPERNVIGTIPQDFLPPTSLKNGDSSYYDPNYLQSNEEKDRFLKIVTKIFNRINDNLSGRILLEELSKANPYLGNDNTPDNQFHIGDASAVEIKFSNGNQSILLPNVIIMGAEPDLFETNSSNISLRNNYMPSNHGFGSIAIVTFSPEYSFRFNDNSMNEFIQDPALTLMHELIHSLHGLYGAKRITTKYTITQQQNPLITNIRGTNIEEFLTFGGTDLNIITSAQYNDIYTNLLADYKKIASKLSKVQVSNPQLNPYKDIFQEKYGLDKNASGIYSVNINKFDDIFKKLYSFTEFDLATKFQVKCRQTYIGQYKYFKLSNLLNNSIYNISEGYNINTLKVNFRGQNTNLNPRIITQLTGRGLVKKIIRFCKNIVFSKGITKSICIEINNGELFFVASENSYNDDNINTPKEIDDTVTSNNNYENDLDQVILNFNSESAPGLSDEKLNLTIQNDAYIPKYDSNGTSYIEQHDVNELNVFFYLDAQKVPEGENNVNLTSSIDTALLEQPKIYTFFSSEFINNVNKTVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKTIIESKYNSYTLEEKNELTNKYNIEQIENELNQKVSIAMNNIEIFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDKKSILNLGNIHVSDNILFKIVNCSYTRYIGIRYFNIFDKELDETEIQTLYNNEPNANILKDFWGNYLLYDKEYYLLNVLKPNNFIDRRTDSTLSINNIRSTILLANRLYSGIKVKIQRVNNSSTNDNLVRKNDQVYINFVASKTHLFPLYADTNTTNKEKTIKSSSSGNRFNQVVVMNSVGNNCTMNFKNNNGNNIGMLGFKDNTLVASTWYYTHMRDNTNSNGCFWNFISEEHGWQEK

SEQ ID NO: 81 - protein, 1251 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFVPELLIPTILVFTIKSFLGSSDNKNKVIKAINNALKERDEKWKEVYSFIVSNWMTKINTQFNKRKEQMYQALQNQVNALKTIIEFKYNSYTLEEKKELKNNYDIEQIENELNQKVSIAMNNIDRFLTESSISYLMKLINEVKINKLREYDENVKTYLLDYIIQHGSILGESQQELNSMVIDTLNNSIPFKLSSYTDDKILISYFNKFFKRIKSSSVLNMRYKNDKYVDTSGYDSNININGEIFIYPTNKNQFTIFNSKPSEVNISQNDYIIYDNKYKNFSISFWVRIPNYDNKIVNINNEYTIINCMRDNNSGWKVSLNHNEIIWTLQDNARINQKLVFKYGNANGISDYINKWIFVTITNDRLGDSKLYINGHLIDQKSILNLGNIHVSDNILFKIVNCSYTRYIGIRYFNIFDKELDETEIQTLYSNEPNTNILKDFWGNYLLYDKGYYLLNVLKPNNFIDRRKDSTLSINNIRSTILLANRLYSGIKVKIQRVNDSSTNDRFVRKNDQVYINYISNSSSYSLYADTNTTDKEKTIKSSSSGNRFNQVVVMNSVGNNCTMNFKNNNGNNIGLLGFKADTVVASTWYYTHMRDHTNSNGCFWNFISEEHGWQEK

SEQ ID NO: 82 - protein, 1252 aa, Clostridium botulinum
MPTINSFNYNDPVNDRTILYIKPGGCQEFYKSFNIMKNIWIIPERNVIGTTPQDFHPPTSLKNGDSSYYDPNYLQSYEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFKDNSMNEFIQDPALTLMHELIHSLHGLYGAKGITTQYTITQQQNPLITNIKGTNIEEFLTFGGTDLNIITSAQYNDIYTNLLADYKKIASKLSKVQVSNPLLNPYKDVFEKKYGLDKDASGIYSVNINKFNDIFKKLYSFTEFDLATKFQVKCRQTYIGQYKYFKLSNLLNNSIYNISEGYNINTLKVNFRGQNTNLNPRIITPLTGRGLVKKIIRFCKNIVFSKGIRKSICIEINNGELFFVASDNSYNDDNINTPKEIDDTVTSNNNYENDLDQVILNFNSESAPGLSDEKLNLTIQNDAYIPKYDSNGTSDIEQHDVNELNVFFYLDAQKVPEGENNIDFTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKTIIESKYNSYTLEEKNELTNKYNIEQIENELNQKVSIAMNNIEIFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNININGDVYKYPTNKNQFGIYNNKLSEVNISQNDYIIYDNKYKNFSISFWVRIPNYDNKIVNVNNEYTIINCMRDNNSGWKVSLNHNEIIWTLQDNSGINQKLAFNYGNANGISDYINKWIFVTITNDRLGDSKLYINGNLIDKKSILNLGNIHVSDNILFKIVNCSYTRYIGIRYFNIFDKELDETEIQTLYNNEPNANILKDFWGNYLLYDKEYYLLNVLKPNNFINRRTDSTLSINNIRSTILLANRLYSGIKVKIQRVNNSSTNDNLVRKNDQVYINFVDSKTHLLPLYADTATTNKEKTIKISSSGNRFNQVVVMNSVGNNCTMNFKNNNGNNIGLLGFKADTVVASTWYYTHMRDNTNSNGFFWNFISEEHGWQEK

SEQ ID NO: 83 - protein, 1252 aa, Clostridium botulinum
MPTINSFNYNDPVNNRTILYIKPGGCQQFYKSFNIMKNIWIIPERNVIGTIPQDFLPPTSLKNGDSSYYDPNYLQSDQEKDKFLKIVTKIFNRINDNLSGRILLEELSKANPYLGNDNTPDGDFIINDASAVPIQFSNGSQSILLPNVIIMGAEPDLFETNSSNISLRNNYMPSNHGFGSIAIVTFSPEYSFRFKDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVGWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKAIIESKYNSYTLEEKNELTNKYDIEQIENELNQKVSIAMNNIDRFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNSGINQKLAFNYGNANGISDYINKWIFVTITNDRLGDSKLYINGNLIDKKSILNLGNIHVSDNILFKIVNCSYTRYIGIRYFNIFDKELDETEIQTLYNNEPNANILKDFWGNYLLYDKEYYLLNVLKPNNFINRRTDSTLSINNIRSTILLANRLYSGIKVKIQRVNNSSTNDNLVRKNDQVYINFVASKTHLLPLYADTATTNKEKTIKISSSGNRFNQVVVMNSVGNNCTMNFKNNNGNNIGLLGFKADTVVASTWYYTHMRDNTNSNGFFWNFISEEHGWQEK

SEQ ID NO: 84 - protein, 1252 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQHILLPNVIIMGAEPDLFETNSSNISLRNNYMPSNHGFGSIAIVTFSPEYSFRFNDNSINEFIQDPALTLMHELIHSLHGLYGAKGITTTCIITQQQNPLITNRKGINIEEFLTFGGNDLNIITVAQYNDIYTNLLNDYRKIASKLSKVQVSNPQLNPYKDIFQEKYGLDKDASGIYSVNINKFDDILKKLYSFTEFDLATKFQVKCRETYIGQYKYFKLSNLLNDSIYNISEGYNINNLKVNFRGQNANLNPRIIKPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 85 - protein, 1252 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGIQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYNNEPNANILKDFWGNYLLYDKEYYLLNVLKPNNFIDRRTDSTLSINNIRSTILLANRLYSGIKVKIQRVNNSSTNDNLVRKNDQVYINFVASKTHLFPLYADTNTTNKEKTIKSSSSGNRFNQVVVMNSVGNNCTMNFKNNNGNNIGMLGFKDNTLVASTWYYTHMRDNTNSNGCFWNFISEEHGWQEK

SEQ ID NO: 86 - protein, 1268 aa, Clostridium botulinum
MPVNINNFNYNDPINNTTILYMKMPYYEDSNKYYKAFEIMDNVWIIPERNIIGKKPSDFYPPISLDSGSSAYYDPNYLTTDAEKDRFLKTVIKLFNRINSNPAGQVLLEEIKNGKPYLGNDHTAVNEFCANNRSTSVEIKESNGTTDSMLLNLVILGPGPNILECSTFPVRIFPNNIAYDPSEKGFGSIQLMSFSTEYEYAFNDNTDLFIADPAISLAHELIHVLHGLYGAKGVTNKKVIEVDQGALMAAEKDIKIEEFITFGGQDLNIITNSTNQKIYDNLLSNYTAIASRLSQVNINNSALNTTYYKNFFQWKYGLDQDSNGNYTVNISKFNAIYKKLFSFTECDLAQKFQVKNRSNYLFHFKPFRLLDLLDDNIYSISEGFNIGSLRVNNNGQNINLNSRIVGPIPDNGLVERFVGLCKSIVSKKGTKNSLCIKVNNRDLFFVASESSYNENGINSPKEIDDTTITNNNYKKNLDEVILDYNSDAIPNLSSRLLNTTAQNDSYVPKYDSNGTSEIKEYTVDKLNVFFYLYAQKAPEGESAISLTSSVNTALLDASKVYTFFSSDFINTVNKPVQAALFISWIQQVINDFTTEATQKSTIDKIADISLVVPYVGLALNIGNEVQKGNFKEAIELLGAGILLEFVPELLIPTILVFTIKSFINSDDSKNKIIKAINNALRERELKWKEVYSWIVSNWLTRINTQFNKRKEQMYQALQNQVDGIKKIIEYKYNNYTLDEKNRLKAEYNIYSIKEELNKKVSLAMQNIDRFLTESSISYLMKLINEAKINKLSEYDKRVNQYLLNYILENSSTLGTSSVQELNNLVSNTLNNSIPFELSEYTNDKILISYFNRFYKRIIDSSILNMKYENNRFIDSSGYGSNISINGDIYIYSTNRNQFGIYSSRLSEVNITQNNTIIYNSRYQNFSVSFWVRIPKYNNLKNLNNEYTIINCMRNNNSGWKISLNYNNIIWTLQDTTGNNQKLVFNYTQMIDISDYINKWTFVTITNNRLGHSKLYINGNLTDQKSILNLGNIHVDDNILFKIVGCNDTRYVGIRYFKIFNMELDKTEIETLYHSEPDSTILKDFWGNYLLYNKKYYLLNLLKPNMSVTKNSDILNINRQRGIYSKTNIFSNARLYTGVEVIIRKVGSTDTSNTDNFVRKNDTVYINVVDGNSEYQLYADVSTSAVEKTIKLRRISNSNYNSNQMIIMDSIGDNCTMNFKTNNGNDIGLLGFHLNNLVASSWYYKNIRNNTRNNGCFWSFISKEHGWQE

SEQ ID NO: 87 - protein, 1277 aa, Clostridium botulinum
MPVEINSFNYDDLVNDNTILYIRPPYYERSNTYFKAFNIMENVWIIPERYRLGIEASKFDPPDSLKAGSDGYFDPNYLSTNTEKNRYLQIMIKLFKRINSNEAGKILLNQIKDAIPYLGNSYTAEDQFTTNNRTISFNVRLANGTIEQEMANLIIWGPGPDLTTNRTGGTTYTPAQSLEAIPYKEGFGSIMTIEFSPEYATAFNDISLTSHAPSLFIKDPALILMHELIHVLHGLYGTYTTGFKIKPNITEPYMEVTKPITSGEFLTFGGNDVNKIPQLIQSQLRSKVLDDYEKIASRLNKVNRATAEINIDKFKYSYQLKYQFVKDSNGVYSVDLDKFNKLYDKIYSFTEFNLAHEFKIKTRNSYLAKNFGPFYLPNLLDNSIYNEADGFNIGDLSVNYKGQVIGSDIDSIKKLEGQGVVSRVVRLCLNSSFKKNTKKPLCITVNNGDLFFIASEDSYGEDTINTPKEIDDTTTLVPSFKNILDKVILDFNKQVTPQIPNRRIRTDIQEDNYIPEYDSNGTSEIEEYNVVDLNAFFYLHAQKVPEGETNISLTSSIDTALSEESKVYTFFSSEFIDTINEPVNAALFIDWISKVIRDFTTEATQKSTVDKIADISLIVPYVGLALNIVNETEKGNFKEAFELLGAGILLEFVPELAIPVILVFTIKSYIDSYENKNKIIKAINNSLIEREAKWKEIYSWIVSNWLTRINTQFNKRKEQMYQALQNQVDAIKTAIEYKYNNYTSDEKNRLESEYNINNIEEELNKKVSLAMKNIERFITESSISYLMKLINEAEVGKLKEYDKRVKRHLLEYIFDYRLILGEQGGELIDLVTSTLNTSIPFELSSYTNDKILIIYFNRLYKKIKDSSILDMRYENNKFIDISGYGSNISINGNVYIYSTNRNQFGIYDDRLSEVNIAQNNDIIYNSRYQNFSISFWVRIPKHYRPMNHNREYTIINCMGNNNSGWKISLRTTGDCEIIWTLQDTSGNKKKLIFRYSQLGGISDYINKWIFVTITNNRLGNSRIYINGNLIVEKSISNLGDIHVSDNILFKIVGCDDKMYVGIRYFKVFNTELDKTEIEILYSNEPDPSILKDYWGNYLLYNKKYYLLNLLRNDKYITRNSDILNISHQRGVTKDLFIFSNYKLYEGVEVIIRKNGPIDISNTDNFVRKNDLAYINVVDHGVEYRLYADISITKPEKIIKLIRRSNPDDSLGQIIVMDSIGNNCTMNFQNNNGGNIGLLGFHSDNLVASSWYYNNIRRNTSSNGCFWSFISKEHGWQE

SEQ ID NO: 88 - protein, 1277 aa, Clostridium botulinum
MPVVINSFNYDDPVNDDTILYMQIPYEEKSKKYYKAFEIMRNVWIMPERNTIGTNPSDFDPPASLKNGSSAYYDPNYLTTDAEKDRYLKTTIKLFKRINSNPAGEVLLQEISYAKPYLGNDHTPINEFHPVTRTTSVNIKSSTNVESSIILNLLVLGAGPNIFENSSYPVRKLMNSGEVYDPSNDGFGSINIVTFSPEYEYTFNDISGGHNSSTESFIADPAISLAHELIHALHGLYGARGVTYKETIKVKQAPLMIAEKPIRLEEFLTFGGQDLNIITSAMKEKIYNDLLANYEKIATRLSEVNSAPPEYDINEYKNYFQWKYGLDKNADGSYTVNENKFNEIYKKLYSFTEIDLANKFKVKCRNTYFIKYGFLKVPNLLDDDIYTVSEGFNIGNLAVNNRGQNINLNPKIIDSIPDKGLVEKIVKLCKSIIPRKGTKAPPRLCIRVNNRELFFVASESSYNENDINTPKEIDDTTNLNNNYRNNLDEVILDYNSETIPQISSQTLNTLVQDDSYVPRYDSNGTSEIEEHNVVDLNAFFYLHAQKVPEGETNISLTSSIDTALSEESKVYTFFSSEFINNINKPVHAALFIGWISQVIRDFTTESTQKSTVDKIADISLIVPYVGLALNIGNDARKGNFKEAFELLGAAILLEVVPELLIPVILVFTIKSFIDSSKNEDKIIKAINNSLIEREAKWKEVYSWIVSNWLTRINTQFNKRKEQMYQALQNQVDAIKTVIEYKYNSYTSDEKNRLESEYNINNIEEELNKKVSLAMKNIERFIAESSISYLMKLINEAKVSELREYDEGVKEYLLDYILKNGSILGDHVQELNDLVTSTLNSSIPFELSSYTNDKILIIYFNKLYKKIKDNCILDMRYENNKFIDISGYGSNISINGELYIYTTNRNQFTIYSGKLSEVNIAQNNDIIYNSRYQNFSISFWVRIPRYSNIVNLNNEYTIINCMGNNNSGWKISLNYNKIIWTLQDTAGNNEKLVFNYTQMISISDYINKWIFVTITNNRLGNSRIYINGNLIDQKSISNLGDIHVSDNILFKIVGCNDTRYVGIRYFKVFDTELDKTEIETLYSDEPDPSILKDFWGNYLLYNKRYYLLNLLRKDNAITQSSTFLSISRARGVDRKANIFSNKRLYKGVEVIIRKNEPIDISNTDNFVRKGDLAYINVVDRDVEYRLYANTSNAQPEKTIKLIRTSNSNDSLDQIIVMDSIGNNCTMNFQNNNGGNIGLLGFHSNTLVASSWYYNNIRRNTSSNGCFWSFISKEHGWQE

SEQ ID NO: 89 - protein, 1280 aa, Clostridium botulinum
MPVVINSFNYNDPVNDETILYMQKPYEERSRKYYKAFEIMPNVWIMPERDTIGTKPDEFQVPDSLKNGSSAYYDPNYLTTDAEKDRYLKTMIKLFNRINSNPTGKVLLEEVSNARPYLGDDDTLINEFFPVNVTTSVNIKFSTDVESSIISNLLVLGAGPDIFKAYCTPLVRFNKSDKLIEPSNHGFGSINILTFSPEYEHIFNDISGGNHNSTESFIADPAISLAHELIHALHGLYGAKAVTHKESLVAERGPLMIAEKPIRLEEFLTFGGEDLNIIPSAMKEKIYNDLLANYEKIATRLREVNTAPPGYDINEYKDYFQWKYGLDRNADGSYTVNRNKFNEIYKKLYSFTEIDLANKFKVKCRNTYFIKYGFVKVPDLLDDDIYTVSEGFNIGNLAVNNRGQNINLNPKIIDSIPDKGLVEKIIKFCKSIIPRKGTKQSPSLCIRVNNRELFFVASESSYNESDINTPKEIDDTTNLNNNYRNNLDEVILDYNSETIPQISNRTLNTLVQDNSYVPRYDSNGTSEIEEYDVVDFNVFFYLHAQKVPEGETNISLTSSIDTALLEESKVYTFFSSEFIDTINKPVNAALFIDWISKVIRDFTTEATQKSTVDKIADISLIVPYVGLALNIVIEAEKGNFEEAFELLGAGILLEFVPELTIPVILVFTIKSYIDSYENKNKAIKAINNSLIEREAKWKEIYSWIVSNWLTRINTQFNKRKEQMYQALQNQVDAIKTAIEYKYNNYTSDEKNRLESKYNINNIEEELNKKVSLAMKNIERFMTESSISYLMKLINEAEVGKLKEYDKHVKSDLLDYILYHKLILGEQTKELIDLVTSTLNSSIPFELSSYTNDKILIIYFNRLYKKIKDSSILDMRYENNKFIDISGYGSNISINGNVYIYSTNRNQFGIYSGRLSEVNIAQNNDIIYNSRYQNFSISFWVRIPKHYRPMNRNREYTIINCMGNNNSGWKISLRTIRDCEIIWTLQDTSGNKEKLIFRYEELASISDYINKWIFVTITNNRLGNSRIYINGNLIVEKSISNLGDIHVSDNILFKIVGCDDETYVGIRYFKVFNTELDKTEIETLYSNEPDPSILKDYWGNYLLYNKKYYLFNLLRKDKYITRNSGILNINQQRGVTGGISVFLNYKLYEGVEVIIRKNAPIDISNTDNFVRKNDLAYINVVDHGVEYRLYADISITKSEKIIKLIRTSNPNDSLGQIIVMDSIGNNCTMNFQNNDGSNIGLLGFHSDDLVASSWYYNHIRRNTSSNGCFWSFISKEHGWKE

SEQ ID NO: 90 - protein, 1279 aa, Clostridium botulinum
MPVVINSFNYNDPVNDETILYMQKPYEERSRKYYKAFEIMPNVWIMPERDTIGTKPDDFQVPDSLKNGSSAYYDPNYLTTDAEKDRYLKTMIKLFNRINSNPTGKVLLEEVSNARPYLGDDDTLINEFFPVNVTTSVNIKFSTDVESSIISNLLVLGAGPDIFKAYCTPLVRFNKSDKLIEPSNHGFGSINILTFSPEYEHIFNDISGGDHNSTESFIADPAISLAHELIHALHGLYGAKAVTHKETIEVKRGPLMIAEKPIRLEEFLTFGGEDLNIIPSAMKEKIYNDLLANYEKIATRLREVNTAPPEYDINEYKDYFQWKYGLDRNADGSYTVNRNKFNGIYKKLYSFTEIDLANKFKVKCRNTYFIKYGFVKVPDLLDDDIYTVSEGFNIGNLAVNNRGQNINLNPKIIDSIPDKGLVEKIIKFCKSIIPRKGTKQSPSLCIRVNNRELFFVASESSYNESDINTPKEIDDTTNLNNNYRNNLDEVILDYNSETIPQISNRTLNTLVQDNSYVPRYDSNGTSEIEEYDVVDFNVFFYLHAQKVPEGETNISLTSSIDTALLEKSKVYTFFSSEFIDTINESVNAALFIDWINKVIRDFTTEATQKSTVDKIADISLIVPYVGLALNIVIDAEKGNFQEAFELLGAGILLEFVPELTIPVILVFTIKSYIDSYENKNKAIKAINNALIEREAKWKEIYSWIVSNWLTKINTQFNKRKEQMYQALQNQVDAIKTAIEYKYNNYTSDEKNRLESEYNINNIEEELNKKVSLAMKNIERFMTESSISYLMKLINEAEVGKLKKYDRHVKSDLLDYILYHKLILGDQTKELIDLVTSTLNSSIPFELSSYTNDKILIIYFNRLYKKIKDSSILDMRYENNKFIDISGYGSNISINGNVYIYSTNRNQFGIYSDRLSEVNIAQNNDIIYNSRYQNFSISFWVRIPKHYGPMNRNREYTIINCMGNNNSGWKISLRNIRDCEIIWTLQDTSGNKEKLIFRYEELANISDYINKWIFVTITNNRLGNSRIYINGNLIVEKSISNLGDIHVSDNILFKIVGCDDKTYVGIRYFKVFNTELDKTEIETLYSNEPDPSILKDYWGNYLLYNKKYYLFNLLRKDKYITRNSGILNINQQRGVTEGSVFLNYKLYEGVEVIIRKNGPIDISNTDNFVRKNDLAYINVVYHDVEYRLYADISITKPEKIIKLIRTSNPNDSLGQIIVMDSIGNNCTMNFQNNNGGNIGLLGFHSDNLVASSWYYNNIRRNTSSNGCFWSFISKEHGWQE

SEQ ID NO: 91 - protein, 1274 aa, Clostridium botulinum
MPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCKSVIPRKGTKAPPRLCIRVNNSELFFVASESSYNENDINTPKEIDDTTNLNNNYRNNLDEVILDYNSQTIPQISNRTLNTLVQDNSYVPRYDSNGTSEIEEYDVVDFNVFFYLHAQKVPEGETNISLTSSIDTALLEESKDIFFSSEFIDTINKPVNAALFIDWISKVIRDFTTEATQKSTVDKIADISLIVPYVGLALNIIIEAEKGNFEEAFELLGVGILLEFVPELTIPVILVFTIKSYIDSYENKNKAIKAINNSLIEREAKWKEIYSWIVSNWLTRINTQFNKRKEQMYQALQNQVDAIKTAIEYKYNNYTSDEKNRLESEYNINNIEEELNKKVSLAMKNIERFMTESSISYLMKLINEAKVGKLKKYDNHVKSDLLNYILDHRSILGEQTNELSDLVTSTLNSSIPFELSSYTNDKILIIYFNRLYKKIKDSSILDMRYENNKFIDISGYGSNISINGNVYIYSTNRNQFGIYNSRLSEVNIAQNNDIIYNSRYQNFSISFWVRIPKHYKPMNHNREYTIINCMGNNNSGWKISLRTVRDCEIIWTLQDTSGNKENLIFRYEELNRISNYINKWIFVTITNNRLGNSRIYINGNLIVEKSISNLGDIHVSDNILFKIVGCDDETYVGIRYFKVFNTELDKTEIETLYSNEPDPSILKNYWGNYLLYNKKYYLFNLLRKDKYITLNSGILNINQQRGVTEGSVFLNYKLYEGVEVIIRKNGPIDISNTDNFVRKNDLAYINVVDRGVEYRLYADTKSEKEKIIRTSNLNDSLGQIIVMDSIGNNCTMNFQNNNGSNIGLLGFHSNNLVASSWYYNNIRRNTSSNGCFWSSISKENGWKE

(19) United States
(12) Patent Application Publication
(10) Pub. No.: US 2026/0125430 A1
(43) Pub. Date: May 7, 2026

(54) CHIMERIC NEUROTOXINS

(71) Applicant: IPSEN BIOPHARM LIMITED, Wrexham (GB)

(72) Inventor: Sai Man LIU, Wrexham (GB)

(73) Assignee: IPSEN BIOPHARM LIMITED, Wrexham (GB)

(21) Appl. No.: 19/309,183

(22) Filed: Aug. 25, 2025

Related U.S. Application Data
(63) Continuation of application No. 17/811,958, filed on Jul. 12, 2022, now Pat. No. 12,421,284.
Application No. 17/811,958 is a division of application No. 16/094,254, now Pat. No. 11,434,265.

(30) Foreign Application Priority Data
May 5, 2016 (GB) 1607901.4

(51) Int. Cl. C07K 14/33; A61K 38/00; C12N 9/52

(52) U.S. Cl. CPC C07K 14/33; C12N 9/52; C12Y 304/24069; A61K 38/00; C07K 2319/55

(57) ABSTRACT

Chimeric neurotoxins comprising an LH_{N} domain from a first neurotoxin covalently linked to an H_{C} domain from a different second neurotoxin are disclosed. The C-terminal residue of the LH_{N} domain corresponds to the first amino acid residue of the 3₁₀ helix separating the LH_{N} and H_{C} domains of the first neurotoxin, and the N-terminal residue of the H_{C} domain corresponds to the second amino acid residue of the corresponding 3₁₀ helix of the second neurotoxin. Also provided are nucleotide sequences encoding such chimeric neurotoxins, vectors and host cells comprising such nucleotide sequences, methods for producing the chimeric neurotoxins, and pharmaceutical compositions and kits containing the same. The chimeric neurotoxins may be used for aesthetic or cosmetic applications.

### FIELD OF THE INVENTION

The present invention relates to chimeric neurotoxins with enhanced properties and their use in therapy.

### SEQUENCE LISTING

The Sequence Listing submitted herewith as an XML file named “17811958CONSL.xml” created on Aug. 25, 2025, having a size of 160,329 bytes, is incorporated herein by reference in its entirety. The Sequence Listing provides the sequences disclosed in the present application in accordance with 37 C.F.R. §§ 1.821-1.825 and conforms to the requirements of WIPO Standard ST.26. The information recorded in the Sequence Listing does not include any new matter and is intended to form part of the present disclosure.

### BACKGROUND

Bacteria in the genus clostridia produce highly potent and specific protein toxins, which can poison neurons and other cells to which they are delivered. Examples of such clostridial toxins include the neurotoxins produced by C. tetani (TeNT) and by C. botulinum (BoNT) serotypes A-G, as well as those produced by C. baratii and C. butyricum.

Among the clostridial neurotoxins are some of the most potent toxins known. By way of example, botulinum neurotoxins have median lethal dose (LD50) values for mice ranging from 0.5 to 5 ng/kg, depending on the serotype. Both tetanus and botulinum toxins act by inhibiting the function of affected neurons, specifically the release of neurotransmitters. While botulinum toxin acts at the neuromuscular junction and inhibits cholinergic transmission in the peripheral nervous system, tetanus toxin acts in the central nervous system.

In nature, clostridial neurotoxins are synthesised as a single-chain polypeptide that is modified post-translationally by a proteolytic cleavage event to form two polypeptide chains joined together by a disulphide bond. Cleavage occurs at a specific cleavage site, often referred to as the activation site, that is located between the cysteine residues that provide the inter-chain disulphide bond. It is this di-chain form that is the active form of the toxin. The two chains are termed the heavy chain (H-chain), which has a molecular mass of approximately 100 kDa, and the light chain (L-chain), which has a molecular mass of approximately 50 kDa. The H-chain comprises an N-terminal translocation component (H_{N} domain) and a C-terminal targeting component (H_{C} domain). The cleavage site is located between the L-chain and the translocation domain components. Following binding of the H_{C} domain to its target neuron and internalisation of the bound toxin into the cell via an endosome, the H_{N} domain translocates the L-chain across the endosomal membrane and into the cytosol, and the L-chain provides a protease function (also known as a non-cytotoxic protease).

Non-cytotoxic proteases act by proteolytically cleaving intracellular transport proteins known as SNARE proteins (e.g. SNAP-25, VAMP, or Syntaxin)—see Gerald K (2002) “Cell and Molecular Biology” (4th edition) John Wiley & Sons, Inc. The acronym SNARE derives from the term Soluble NSF Attachment Receptor, where NSF means N-ethylmaleimide-Sensitive Factor. SNARE proteins are integral to intracellular vesicle fusion, and thus to secretion of molecules via vesicle transport from a cell. The protease function is a zinc-dependent endopeptidase activity and exhibits a high substrate specificity for SNARE proteins.

Accordingly, once delivered to a desired target cell, the non-cytotoxic protease is capable of inhibiting cellular secretion from the target cell. The L-chain proteases of clostridial neurotoxins are non-cytotoxic proteases that cleave SNARE proteins.

In view of the ubiquitous nature of SNARE proteins, clostridial neurotoxins such as botulinum toxin have been successfully employed in a wide range of therapies.

By way of example, we refer to William J. Lipham, Cosmetic and Clinical Applications of Botulinum Toxin (Slack, Inc., 2004), which describes the use of clostridial neurotoxins, such as botulinum neurotoxins (BoNTs), BoNT/A, BoNT/B, BoNT/C1, BoNT/D, BoNT/E, BoNT/F and BoNT/G, and tetanus neurotoxin (TeNT), to inhibit neuronal transmission in a number of therapeutic and cosmetic or aesthetic applications—for example, marketed botulinum toxin products are currently approved as therapeutics for indications including focal spasticity, upper limb spasticity, lower limb spasticity, cervical dystonia, blepharospasm, hemifacial spasm, hyperhidrosis of the axillae, chronic migraine, neurogenic detrusor overactivity, glabellar lines, and severe lateral canthal lines. In addition, clostridial neurotoxin therapies are described for treating neuromuscular disorders (see U.S. Pat. No. 6,872,397); for treating uterine disorders (see US 2004/0175399); for treating ulcers and gastroesophageal reflux disease (see US 2004/0086531); for treating dystonia (see U.S. Pat. No. 6,319,505); for treating eye disorders (see US 2004/0234532); for treating blepharospasm (see US 2004/0151740); for treating strabismus (see US 2004/0126396); for treating pain (see U.S. Pat. Nos. 6,869,610, 6,641,820, 6,464,986, and 6,113,915); for treating fibromyalgia (see U.S. Pat. No. 6,623,742, US 2004/0062776); for treating lower back pain (see US 2004/0037852); for treating muscle injuries (see U.S. Pat. No. 6,423,319); for treating sinus headache (see U.S. Pat. No. 6,838,434); for treating tension headache (see U.S. Pat. No. 6,776,992); for treating headache (see U.S. Pat. No. 6,458,365); for reduction of migraine headache pain (see U.S. Pat. No. 5,714,469); for treating cardiovascular diseases (see U.S. Pat. No. 6,767,544); for treating neurological disorders such as Parkinson's disease (see U.S. Pat. Nos. 6,620,415, 6,306,403); for treating neuropsychiatric disorders (see US 2004/0180061, US 2003/0211121); for treating endocrine disorders (see U.S. Pat. No. 6,827,931); for treating thyroid disorders (see U.S. Pat. No. 6,740,321); for treating cholinergic influenced sweat gland disorders (see U.S. Pat. No. 6,683,049); for treating diabetes (see U.S. Pat. Nos. 6,337,075, 6,416,765); for treating a pancreatic disorder (see U.S. Pat. Nos. 6,261,572, 6,143,306); for treating cancers such as bone tumors (see U.S. Pat. Nos. 6,565,870, 6,368,605, 6,139,845, US 2005/0031648); for treating otic disorders (see U.S. Pat. Nos. 6,358,926, 6,265,379); for treating autonomic disorders such as gastrointestinal muscle disorders and other smooth muscle dysfunction (see U.S. Pat. No. 5,437,291); for treatment of skin lesions associated with cutaneous cell-proliferative disorders (see U.S. Pat. No. 5,670,484); for management of neurogenic inflammatory disorders (see U.S. Pat. No. 6,063,768); for reducing hair loss and stimulating hair growth (see U.S. Pat. No. 6,299,893); for treating downturned mouth (see U.S. Pat. No. 6,358,917); for reducing appetite (see US 2004/40253274); for dental therapies and procedures (see US 2004/0115139); for treating neuromuscular disorders and conditions (see US 2002/0010138); for treating various disorders and conditions and associated pain (see US 2004/0013692); for treating conditions resulting from mucus hypersecretion such as asthma and COPD (see WO 00/10598); and for treating non-neuronal conditions such as inflammation, endocrine conditions, exocrine conditions, immunological conditions, cardiovascular conditions, bone conditions (see WO 01/21213). All of the above publications are hereby incorporated by reference in their entirety.

The use of non-cytotoxic proteases such as clostridial neurotoxins (e.g. BoNTs and TeNT) in therapeutic and cosmetic treatments of humans and other mammals is anticipated to expand to an ever-widening range of diseases and ailments that can benefit from the properties of these toxins.

Currently all approved drugs/cosmetic preparations comprising BoNTs contain naturally occurring neurotoxins purified from clostridial strains (BoNT/A in the case of DYSPORT®, BOTOX® or XEOMIN®, and BoNT/B in the case of MYOBLOC®).

Recombinant technology offers the possibility of changing or optimizing the properties of neurotoxins through the introduction of modifications to its sequence and/or structure. In particular, chimeric neurotoxins in which the H_{C} domain or the H_{CC} subdomain is replaced by a H_{C} domain or H_{CC} subdomain from a different neurotoxin have been produced.

Rummel et al, 2011 (Exchange of the H_{CC} domain mediating double receptor recognition improves the pharmacodynamic properties of botulinum neurotoxin. FEBS Journal, 278(23), 4506-4515) generated various active full-length hybrid neurotoxins, including AABB, AACC and BBAA chimera (letters represent the serotype origin of each of the four domains: L, H_{N}, H_{CN}, H_{CC}). The AABB chimera was found to be more potent than BoNT/A in a mouse phrenic nerve hemidiaphragm assay, while the AACC only retained 10% of the potency of BoNT/A. The BBAA chimera retained 85% of the potency of BoNT/A and was equipotent to BoNT/B.

Wang et al, 2008 (Novel chimeras of botulinum neurotoxins A and E unveil contributions from the binding, translocation, and protease domains to their functional characteristics. Journal of Biological Chemistry, 283(25), 16993-17002) generated AE (LH_{N} from BoNT/A and H_{C} from BoNT/E) and EA (LH_{N} from BoNT/E and H_{C} from BoNT/A) chimeric neurotoxins, adding a linker in the case of the AE chimera between the LH_{N} and H_{C} domains to increase flexibility.

Both were able to cause a paralysis in a mouse phrenic nerve hemidiaphragm assay as well as in vivo.

Wang et al., 2012a (Longer-acting and highly potent chimaeric inhibitors of excessive exocytosis created with domains from botulinum neurotoxin A and B. Biochemical Journal, 444(1), 59-67) generated AB (LH_{N} from BoNT/A and H_{C} from BoNT/B, with a linker to improve folding) and BA (LH_{N} from BoNT/B and H_{C} from BoNT/A) chimeric neurotoxins. The AB chimera induced a more prolonged neuromuscular paralysis than BoNT/A in mice. The BA chimera was able to reduce exocytosis from non-neuronal cells.

Wang et al, 2012b (Novel chimeras of botulinum and tetanus neurotoxins yield insights into their distinct sites of neuroparalysis. The FASEB Journal, 26(12), 5035-5048) generated ATx (LH_{N} from BoNT/A and H_{C} from TeNT), TxA (LH_{N} from TeNT and H_{C} from BoNT/A), ETx (LH_{N} from BoNT/E and H_{C} from TeNT) and TxE (LH_{N} from TeNT and H_{C} from BoNT/E) chimera. The information provided with respect to the protein sequence of these prior art chimeric neurotoxins is summarised in table 1 below:

TABLE 1
LH_{N} and H_{C} domains in prior art chimeric neurotoxins

| | Chimera | LH_{N} | H_{C} |
|---|---|---|---|
| Rummel, | AABB | A | B: 871-1304 |
| 2011 | AACC | A | C: 871-1296 |
| | BBAA | B | A: 858-1283 |
| Wang, 2008 | AE | A: 1-874 (+ELGGGGSEL linker) | E: 845-1252 |
| | EA | E: 1-844 (+DI linker) | A: 871-1296 |
| Wang, | AB | A: 1-874 (+ELGGGGSEL linker) | B: 858-1283 |
| 2012(a) | BA | B: 1-861 (+DI linker) | A: 871-1296 |
| Wang, | ATx | A: 1-877 | Tx: 879-1315 |
| 2012(b) | TxA | Tx: 1-882 (+DI linker) | A: 871-1296 |
| | ETx | E: 1-844 (+DI linker) | Tx: 879-1315 |
| | TxE | Tx: 1-882 (+EL linker) | E: 845-1252 |

However, there still exists a need for an optimized design of chimeric neurotoxins allowing for improved therapeutic properties.

The present invention solves the above problem by providing chimeric neurotoxins, as specified in the claims.

### SUMMARY OF THE INVENTION

In one aspect, the invention provides a chimeric neurotoxin comprising a LH_{N} domain from a first neurotoxin covalently linked to a H_{C} domain from a second neurotoxin, wherein the first and second neurotoxins are different, wherein the C-terminal amino acid residue of the LH_{N} domain corresponds to the first amino acid residue of the 3₁₀ helix separating the LH_{N} and H_{C} domains in the first neurotoxin, and wherein the N-terminal amino acid residue of the H_{C} domain corresponds to the second amino acid residue of the 3₁₀ helix separating the LH_{N} and H_{C} domains in the second neurotoxin.

In a second aspect, the invention provides a nucleotide sequence encoding a chimeric neurotoxin according to the invention.

In a third aspect, the invention provides a vector comprising a nucleotide sequence according to the invention.

In a fourth aspect, the invention provides a cell comprising a nucleotide sequence or a vector according to the invention.

In a fifth aspect, the invention provides a pharmaceutical composition comprising a chimeric neurotoxin according to the invention.

In a sixth aspect, the invention provides a chimeric neurotoxin according to the invention for use in therapy.

In a seventh aspect, the invention provides a non-therapeutic use of a chimeric neurotoxin according to the invention for treating an aesthetic or cosmetic condition.

### DETAILED DESCRIPTION

In one aspect, the invention provides a chimeric neurotoxin comprising a LH_{N} domain from a first neurotoxin covalently linked to a H_{C} domain from a second neurotoxin, wherein the first and second neurotoxins are different,
wherein the C-terminal amino acid residue of the LH_{N} domain corresponds to the first amino acid residue of the 3₁₀ helix separating the LH_{N} and H_{C} domains in the first neurotoxin, and
wherein the N-terminal amino acid residue of the H_{C} domain corresponds to the second amino acid residue of the 3₁₀ helix separating the LH_{N} and H_{C} domains in the second neurotoxin.

As used herein, the term “a”, “an” and “the” can mean one or more.

The term “neurotoxin” as used herein means any polypeptide that enters a neuron and inhibits neurotransmitter release. This process encompasses the binding of the neurotoxin to a low or high affinity receptor, the internalisation of the neurotoxin, the translocation of the endopeptidase portion of the neurotoxin into the cytoplasm and the enzymatic modification of the neurotoxin substrate. More specifically, the term “neurotoxin” encompasses any polypeptide produced by Clostridium bacteria (clostridial neurotoxins) that enters a neuron and inhibits neurotransmitter release, and such polypeptides produced by recombinant technologies or chemical techniques. It is this di-chain form that is the active form of the toxin. The two chains are termed the heavy chain (H-chain), which has a molecular mass of approximately 100 kDa, and the light chain (L-chain), which has a molecular mass of approximately 50 kDa. Preferably, the first and second neurotoxins are clostridial neurotoxins.

An example of a BoNT/A neurotoxin amino acid sequence is provided as SEQ ID NO: 1 (UNIPROT® accession number A5HZZ9). An example of a BoNT/B neurotoxin amino acid sequence is provided as SEQ ID NO: 2 (UNIPROT® accession number B1INP5). An example of a BoNT/C neurotoxin amino acid sequence is provided as SEQ ID NO: 3 (UNIPROT® accession number P18640). An example of a BoNT/D neurotoxin amino acid sequence is provided as SEQ ID NO: 4 (UNIPROT® accession number P19321). An example of a BoNT/E neurotoxin amino acid sequence is provided as SEQ II) NO: 5 (UNIPROT® accession number Q00496). An example of a BoNT/F neurotoxin amino acid sequence is provided as SEQ ID NO: 6 (UNIPROT® accession number Q57236). An example of a BoNT/G neurotoxin amino acid sequence is provided as SEQ ID NO: 7 (UNIPROT® accession number Q60393). An example of a TeNT neurotoxin amino acid sequence is provided as SEQ ID NO: 8 (UNIPROT® accession number P04958). The amino acid sequences of said neurotoxins are shown in the alignment of FIG. 1 below, along with the sequences of other neurotoxins (i.e. SEQ ID NOs: 58 to 91).

The term “chimeric neurotoxin” as used herein means a neurotoxin comprising or consisting of an LH_{N} domain originating from a first neurotoxin and a H_{C} domain originating from a second neurotoxin.

The term “H_{C} domain” as used herein means a functionally distinct region of the neurotoxin heavy chain with a molecular weight of approximately 50 kDa that enables the binding of the neurotoxin to a receptor located on the surface of the target cell. The H_{C} domain consists of two structurally distinct subdomains, the “H_{CN} subdomain” (N-terminal part of the H_{C} domain) and the “H_{CC} subdomain” (C-terminal part of the H_{C} domain), each of which has a molecular weight of approximately 25 kDa.

The term “LH_{N} domain” as used herein means a neurotoxin that is devoid of the H_{C} domain and consists of an endopeptidase domain (“L” or “light chain”) and the domain responsible for translocation of the endopeptidase into the cytoplasm (H_{N} domain of the heavy chain).

Reference herein to the “first amino acid residue of the 3₁₀ helix separating the LH_{N} and H_{C} domains in the first neurotoxin” means the N-terminal residue of the 3₁₀ helix separating the LH_{N} and H_{C} domains.

Reference herein to the “second amino acid residue of the 3₁₀ helix separating the LH_{N} and H_{C} domains in the second neurotoxin” means the amino acid residue following the N-terminal residue of the 3₁₀ helix separating the LH_{N} and H_{C} domains.

A “3₁₀ helix” is a type of secondary structure found in proteins and polypeptides, along with α-helices, β-sheets and reverse turns. The amino acids in a 3₁₀ helix are arranged in a right-handed helical structure where each full turn is completed by three residues and ten atoms that separate the intramolecular hydrogen bond between them. Each amino acid corresponds to a 120° turn in the helix (i.e., the helix has three residues per turn), and a translation of 2.0 Å (=0.2 nm) along the helical axis, and has 10 atoms in the ring formed by making the hydrogen bond. Most importantly, the N—H group of an amino acid forms a hydrogen bond with the C═O group of the amino acid three residues earlier; this repeated i+3→i hydrogen bonding defines a 3₁₀ helix. A 3₁₀ helix is a standard concept in structural biology with which the skilled person is familiar.

This 3₁₀ helix corresponds to four residues which form the actual helix and two cap (or transitional) residues, one at each end of these four residues. The term “3₁₀ helix separating the LH_{N} and H_{C} domains” as used herein consists of those 6 residues.

Through carrying out structural analyses and sequence alignments, the inventor identified a 3₁₀ helix separating the LH_{N} and H_{C} domains in tetanus and botulinum neurotoxins. This 3₁₀ helix is surrounded by an α-helix at its N-terminus (i.e. at the C-terminal part of the LH_{N} domain) and by a β-strand at its C-terminus (i.e. at the N-terminal part of the H_{C} domain). The first (N-terminal) residue (cap or transitional residue) of the 3₁₀ helix also corresponds to the C-terminal residue of this α-helix.

The 3₁₀ helix separating the LH_{N} and H_{C} domains can be for example determined from publically available crystal structures of botulinum neurotoxins, for example 3BTA and 1EPW from RCSB Protein Data Bank for botulinum neurotoxins A1 and B1 respectively.

In silico modelling and alignment tools which are publically available can also be used to determine the location of the 3₁₀ helix separating the LH_{N} and H_{C} domains in other neurotoxins, for example the homology modelling servers LOOPP (Learning, Observing and Outputting Protein Patterns), PHYRE (Protein Homology/analogY Recognition Engine) and Rosetta, the protein superposition server SuperPose, the alignment program Clustal Omega, and a number of other tools/services listed at the Internet Resources for Molecular and Cell Biologists. The inventor found in particular that the region around the “H_{N}/H_{CN}” junction is structurally highly conserved which renders it an ideal region to superimpose different serotypes.

For example, the following methodology was used by the inventor to determine the sequence of this 3₁₀ helix in other neurotoxins:
1. The structural homology modelling tool LOOP was used to obtain a predicted structure of all BoNT serotypes and TeNT based on the BoNT/A1 crystal structure (3BTA.pdb);
2. The structural (pdb) files thus obtained were edited to include only the N-terminal end of the H_{CN} domain and about 80 residues before it (which are part of the H_{N} domain), thereby retaining the “H_{N}/H_{CN}” region which is structurally highly conserved;
3. The protein superposition server SuperPose was used to superpose each serotype onto the 3BTA.pdb structure:
4. The superposed pdb files were inspected to locate the 3₁₀ helix at the start of the H_{C} domain of BoNT/A1, and corresponding residues in the other serotype were then identified.
5. All BoNT serotype sequences were aligned with Clustal Omega in order to check that corresponding residues were correct.

Examples of LH_{N}, H_{C} and 3₁₀ helix domains determined by this method are presented in table 2.

TABLE 2
LH_{N}, H_{C} and 3₁₀ helix domains

| | | | | | SEQ ID |
|---|---|---|---|---|---|
| | | | | | NO |
| Neuro- | Accession | | | | (3₁₀ |
| toxin | Number | LH_{N} | H_{C} | 3₁₀ helix | helix) |
| BoNT/A1 | A5HZZ9 | 1-872 | 873-1296 | ⁸⁷²NIINTS⁸⁷⁷ | 14 |
| BoNT/A2 | X73423 | 1-872 | 873-1296 | ⁸⁷²NIVNTS⁸⁷⁷ | 15 |
| BoNT/A3 | DQ185900 | 1-872 | 873-1292 | ⁸⁷²NIVNTS⁸⁷⁷ | 16 |
| BoNT/A4 | EU341307 | 1-872 | 873-1296 | ⁸⁷²NITNAS⁸⁷⁷ | 17 |
| BoNT/A5 | EU679004 | 1-872 | 873-1296 | ⁸⁷²NIINTS⁸⁷⁷ | 18 |
| BoNT/A6 | FJ981696 | 1-872 | 873-1296 | ⁸⁷²NIINTS⁸⁷⁷ | 19 |
| BoNT/A7 | JQ954969 | 1-872 | 873-1296 | ⁸⁷²NIINTS⁸⁷⁷ | 20 |
| BoNT/A8 | KM233166 | 1-872 | 873-1297 | ⁸⁷²NITNTS⁸⁷⁷ | 21 |
| BoNT/B1 | B1INP5 | 1-859 | 860-1291 | ⁸⁵⁹EILNNI⁸⁶⁴ | 22 |
| BoNT/B2 | AB084152 | 1-859 | 860-1291 | ⁸⁵⁹EILNNI⁸⁶⁴ | 23 |
| BoNT/B3 | EF028400 | 1-859 | 860-1291 | ⁸⁵⁹EILNNI⁸⁶⁴ | 24 |
| BoNT/B4 | EF051570 | 1-859 | 860-1291 | ⁸⁵⁹EILNNI⁸⁶⁴ | 25 |
| BoNT/B5 | EF033130 | 1-859 | 860-1291 | ⁸⁵⁹DILNNI⁸⁶⁴ | 26 |
| BoNT/B6 | AB302852 | 1-859 | 860-1291 | ⁸⁵⁹EILNNI⁸⁶⁴ | 27 |
| BoNT/B7 | JQ354985 | 1-859 | 860-1291 | ⁸⁵⁹EILNNI⁸⁶⁴ | 28 |
| BoNT/B8 | JQ964806 | 1-859 | 860-1292 | ⁸⁵⁹EILNNI⁸⁶⁴ | 29 |
| BoNT/C1 | P18640 | 1-867 | 868-1291 | ⁸⁶⁷NINDSK⁸⁷² | 30 |
| BoNT/CD | AB200360 | 1-867 | 868-1280 | ⁸⁶⁷SINDSK⁸⁷² | 31 |
| BoNT/DC | AB745660 | 1-863 | 864-1276 | ⁸⁶³SINDSK⁸⁶⁸ | 32 |
| BoNT/D | P19321 | 1-863 | 864-1276 | ⁸⁶³SINDSK⁸⁶⁸ | 33 |
| BoNT/E1 | Q00496 | 1-846 | 847-1252 | ⁸⁴⁶RIKSSS⁸⁵¹ | 34 |
| BoNT/E2 | EF028404 | 1-846 | 847-1252 | ⁸⁴⁶RIKSSS⁸⁵¹ | 35 |
| BoNT/E3 | EF028403 | 1-846 | 847-1252 | ⁸⁴⁶RIKSSS⁸⁵¹ | 36 |
| BoNT/E4 | AB088207 | 1-846 | 847-1252 | ⁸⁴⁶RIKSSS⁸⁵¹ | 37 |
| BoNT/E5 | AB037711 | 1-846 | 847-1251 | ⁸⁴⁶RIKSSS⁸⁵¹ | 38 |
| B0NT/E6 | AM695759 | 1-846 | 847-1252 | ⁸⁴⁶RIKSSS⁸⁵¹ | 39 |
| BoNT/E7 | JN695729 | 1-846 | 847-1252 | ⁸⁴⁶RIKSSS⁸⁵¹ | 40 |
| BoNT/E8 | JN695730 | 1-846 | 847-1252 | ⁸⁴⁶RIKSSS⁸⁵¹ | 41 |
| BoNT/E9 | JX424534 | 1-846 | 847-1251 | ⁸⁴⁶RIKSSS⁸⁵¹ | 42 |
| BoNT/E10 | KF861917 | 1-846 | 847-1252 | ⁸⁴⁶RIKSSS⁸⁵¹ | 43 |
| BoNT/E11 | KF861875 | 1-846 | 847-1252 | ⁸⁴⁶RIKSSS⁸⁵¹ | 44 |
| BoNT/E12 | KM370319 | 1-846 | 847-1254 | ⁸⁴⁶RIKSSS⁸⁵¹ | 45 |
| BoNT/F1 | Q57236 | 1-865 | 866-1278 | ⁸⁶⁵KIKDNS⁸⁷⁰ | 46 |
| BoNT/F2 | GU213209 | 1-865 | 866-1280 | ⁸⁶⁵KIKDSS⁸⁷⁰ | 47 |
| BoNT/F3 | GU213227 | 1-865 | 866-1279 | ⁸⁶⁵KIKDSS⁸⁷⁰ | 48 |
| BoNT/F4 | GU213214 | 1-865 | 866-1277 | ⁸⁶⁵KIKDNC⁸⁷⁰ | 49 |
| BoNT/F5 | GU213211 | 1-862 | 863-1277 | ⁸⁶²KIKDSS⁸⁶⁷ | 50 |
| B0NT/F6 | M92906 | 1-864 | 865-1274 | ⁸⁶⁴KIKDSS⁸⁶⁹ | 51 |
| BoNT/F7 | GU213233 | 1-856 | 857-1268 | ⁸⁵⁶KIKDSS⁸⁶¹ | 52 |
| BoNT/G | Q60393 | 1-864 | 865-1297 | ⁸⁶⁴NISSNA⁸⁶⁹ | 53 |
| BoNT/H | KG015617 | 1-860 | 861-1288 | ⁸⁶⁰ELKYNC⁸⁶⁵ | 54 |
| TeNT | P04958 | 1-880 | 881-1315 | ⁸⁸⁰ILKKST⁸⁸⁵ | 55 |

Using structural analysis and sequence alignments, the inventor found that the β-strand following the 3₁₀ helix separating the LH_{N} and H_{C} domains is a conserved structure in all botulinum and tetanus neurotoxins and starts at the 8^{th} residue when starting from the first residue of the 3₁₀ helix separating the LH_{N} and H_{C} domains (e.g., at residue 879 for BoNT/A1).

According to an alternative definition, the first aspect of the invention provides a chimeric neurotoxin comprising an LH_{N} domain from a first neurotoxin covalently linked to a H_{C} domain from a second neurotoxin, wherein the first and second neurotoxins are different,
wherein the C-terminal amino acid residue of the LH_{N} domain corresponds to the eighth amino acid residue N-terminally to the β-strand located at the beginning (N-term) of the H_{C} domain in the first neurotoxin, and
wherein the N-terminal amino acid residue of the H_{C} domain corresponds to the seventh amino acid residue N-terminally to the β-strand located at the beginning (N-term) of the H_{C} domain in the second neurotoxin.

According to yet another definition, the first aspect of the invention provides a chimeric neurotoxin comprising a LH_{N} domain from a first neurotoxin covalently linked to a H_{C} domain from a second neurotoxin, wherein the first and second neurotoxins are different,
wherein the C-terminal amino acid residue of the LH_{N} domain corresponds to the C-terminal amino acid residue of the α-helix located at the end (C-term) of LH_{N} domain in the first neurotoxin, and
wherein the N-terminal amino acid residue of the H_{C} domain corresponds to the amino acid residue immediately C-terminal to the C-terminal amino acid residue of the α-helix located at the end (C-term) of LH_{N} domain in the second neurotoxin.

The rationale of the design process of the chimeric neurotoxins according to the invention was to try to ensure that the secondary structure was not compromised and thereby minimise any changes to the tertiary structure and to the function of each domain.

In some of the prior art chimeric neurotoxins, a linker is required between the LH_{N} and H_{C} domains (see table 1), presumably to ensure acceptable expression and purification.

Without wishing to be bound by theory, it is hypothesized that structuring chimeric neurotoxins in the form of proteins which have a tertiary structure closely mimicking the tertiary structure of natural neurotoxins will facilitate their solubility.

Without wishing to be bound by theory, it is further hypothesized that the fact of not disrupting the four central amino acid residues of the 3₁₀ helix in the chimeric neurotoxin ensures an optimal conformation for the chimeric neurotoxin, thereby allowing for the chimeric neurotoxin to exert its functions to their full capacity.

In fact, the inventor has surprisingly found that retaining solely the first amino acid residue of the 3₁₀ helix of the first neurotoxin and the second amino acid residue of the 3₁₀ helix onwards of second neurotoxin not only allows the production of soluble and functional chimeric neurotoxins, but further leads to improved properties over other chimeric neurotoxins, in particular an increased potency, an increased safety ratio and/or a longer duration of action.

Undesired effects of a neurotoxin (caused by diffusion of the neurotoxin away from the site of administration) can be assessed experimentally by measuring percentage bodyweight loss in a relevant animal model (e.g. a mouse, where loss of bodyweight is detected within seven days of administration). Conversely, desired on-target effects of a neurotoxin can be assessed experimentally by Digital Abduction Score (DAS) assay, a measurement of muscle paralysis.

The DAS assay may be performed by injection of 20 μL of neurotoxin, formulated in Gelatin Phosphate Buffer, into the mouse gastrocnemius/soleus complex, followed by assessment of Digital Abduction Score using the method of Aoki (Aoki KR, Toxicon 39: 1815-1820; 2001). In the DAS assay, mice are suspended briefly by the tail in order to elicit a characteristic startle response in which the mouse extends its hind limbs and abducts its hind digits. Following neurotoxin injection, the varying degrees of digit abduction are scored on a five-point scale (0=normal to 4=maximal reduction in digit abduction and leg extension).

The Safety Ratio of a neurotoxin may then be expressed as the ratio between the amount of neurotoxin required for a 10% drop in a bodyweight of a mouse (measured at peak effect within the first seven days after dosing in a mouse) and the amount of neurotoxin required for a DAS score of 2. High Safety Ratio scores are therefore desired, and indicate a neurotoxin that is able to effectively paralyze a target muscle with little undesired off-target effects.

A high safety ratio is particularly advantageous in therapy because it represents an increase in the therapeutic index. In other words, this means that reduced dosages can be used compared to known clostridial toxin therapeutics and/or that increased dosages can be used without any additional effects. The possibility to use higher doses of neurotoxin without additional effects is particularly advantageous as higher doses usually lead to a longer duration of action of the neurotoxin.

The Potency of a neurotoxin may be expressed as the minimal dose of neurotoxin which leads to a given DAS score when administered to a mouse gastrocnemius/soleus complex, for example a DAS score of 2 (ED₅₀ dose) or a DAS score of 4. The Potency of a neurotoxin may be also expressed as the EC₅₀ dose in a cellular assay measuring SNARE cleavage by the neurotoxin, for example the EC₅₀ dose in a cellular assay measuring SNAP-25 cleavage by a chimeric BoNT/AB neurotoxin.

The duration of action of a neurotoxin may be expressed as the time required for retrieving a DAS score of 0 after administration of a given dose of neurotoxin, for example the minimal dose of neurotoxin leading to a DAS score of 4, to a mouse gastrocnemius/soleus complex.

In one embodiment, the first neurotoxin is a Botulinum Neurotoxin (BoNT) serotype A, serotype B, serotype C, serotype D, serotype E, serotype F or serotype G or a Tetanus Neurotoxin (TeNT), and the second neurotoxin is a Botulinum Neurotoxin (BoNT) serotype A, serotype B, serotype C, serotype D, serotype E, serotype F or serotype G or a Tetanus Neurotoxin (TeNT). In a preferred embodiment, the first neurotoxin is a Botulinum Neurotoxin (BoNT) serotype A, serotype B or a serotype C, and the second neurotoxin is a Botulinum Neurotoxin (BoNT) serotype A, serotype B or a serotype C.

Different BoNT serotypes can be distinguished based on inactivation by specific neutralising anti-sera, with such classification by serotype correlating with percentage sequence identity at the amino acid level. BoNT proteins of a given serotype are further divided into different subtypes on the basis of amino acid percentage sequence identity.

Preferably, the first and second neurotoxins are Botulinum Neurotoxins from different serotypes. In another embodiment, either the first or second neurotoxin is a Botulinum Neurotoxin and the other neurotoxin is a Tetanus neurotoxin.

Using an “XY” representation according to which X is the LH_{N} domain and Y is the H_{C} domain, the following chimeric neurotoxins are embodiments of the present invention:
AB, AC, AD, AE, AF, AG, ATx,
BA, BC, BD, BE, BF, BG, BTx,
CA, CB, CD, CE, CF, CG, CTx,
DA, DB, DC, DE, DF, DG, DTx,
EA, EB, EC, ED, EF, EG, ETx,
FA, FB, FC, FD, FE, FG, FTx,
GA, GB, GC, GD, GE, GF, FTx,
TxA, TxB, TxC, TxD, TxE, TxF, TxG,
wherein A, B, C, D, E, F, G and Tx are respectively Botulinum Neurotoxin (BoNT) serotype A, serotype B, serotype C, serotype D, serotype E, serotype F, serotype G and Tetanus Neurotoxin (TeNT).

Yet, using the same “XY” representation as described above, the following chimeric neurotoxins are preferred embodiments of the present invention:
AB, AC,
BA, BC,
CA, CB,
wherein A, B, C, are respectively Botulinum Neurotoxin (BoNT) serotype A, serotype B, and serotype C.

In one embodiment, the LH_{N} domain from the first neurotoxin corresponds to:
amino acid residues 1 to 872 of SEQ ID NO: 1, or a polypeptide sequence having at least 70% sequence identity thereto,
amino acid residues 1 to 859 of SEQ ID NO: 2, or a polypeptide sequence having at least 70% sequence identity thereto,
amino acid residues 1 to 867 of SEQ ID NO: 3, or a polypeptide sequence having at least 70% sequence identity thereto,
amino acid residues 1 to 863 of SEQ ID NO: 4, or a polypeptide sequence having at least 70% sequence identity thereto,
amino acid residues 1 to 846 of SEQ ID NO: 5, or a polypeptide sequence having at least 70% sequence identity thereto,
amino acid residues 1 to 865 of SEQ ID NO: 6, or a polypeptide sequence having at least 70% sequence identity thereto,
amino acid residues 1 to 864 of SEQ ID NO: 7, or a polypeptide sequence having at least 70% sequence identity thereto G, or
amino acid residues 1 to 880 of SEQ ID NO: 8, or a polypeptide sequence having at least 70% sequence identity thereto.
and the H_{C} domain from the second neurotoxin corresponds to:
   amino acid residues 873 to 1296 of SEQ ID NO: 1, or a polypeptide sequence having at least 70% sequence identity thereto,
   amino acid residues 860 to 1291 of SEQ ID NO: 2, or a polypeptide sequence having at least 70% sequence identity thereto,
   amino acid residues 868 to 1291 of SEQ ID NO: 3, or a polypeptide sequence having at least 70% sequence identity thereto,
   amino acid residues 864 to 1276 of SEQ ID NO: 4, or a polypeptide sequence having at least 70% sequence identity thereto,
   amino acid residues 847 to 1251 of SEQ ID NO: 5, or a polypeptide sequence having at least 70% sequence identity thereto,
   amino acid residues 866 to 1275 of SEQ ID NO: 6, or a polypeptide sequence having at least 70% sequence identity thereto,
   amino acid residues 865 to 1297 of SEQ ID NO: 7, or a polypeptide sequence having at least 70% sequence identity thereto, or
   amino acid residues 881 to 1315 of SEQ ID NO: 8, or a polypeptide sequence having at least 70% sequence identity thereto.

The “percent sequence identity” between two or more nucleic acid or amino acid sequences is a function of the number of identical nucleotides/amino acids at identical positions shared by the aligned sequences. Thus, % identity may be calculated as the number of identical nucleotides/amino acids at each position in an alignment divided by the total number of nucleotides/amino acids in the aligned sequence, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, in particular a global alignment mathematical algorithm (Needleman and Wunsch, J. Mol. Biol. 48(3), 443-453, 1972) such as BLAST, which will be familiar to a skilled person.

The first or second neurotoxin can be a mosaic neurotoxin. The term “mosaic neurotoxin” as used in this context refers to a naturally occurring clostridial neurotoxin that comprises at least one functional domain from another type of clostridial neurotoxins (e.g. a clostridial neurotoxin of a different serotype), said clostridial neurotoxin not usually comprising said at least one functional domain. Examples of mosaic neurotoxins are naturally occurring BoNT/DC and BoNT/CD. BoNT/DC comprises the L chain and H_{N} domain of serotype D and the H_{C} domain of serotype C, whereas BoNT/CD consists of the L chain and H_{N} domain of serotype C and the H_{C} domain of serotype D.

The first and second neurotoxins can be modified neurotoxins and derivatives thereof, including but not limited to those described below. A modified neurotoxin or derivative may contain one or more amino acids that has been modified as compared to the native (unmodified) form of the neurotoxin, or may contain one or more inserted amino acids that are not present in the native (unmodified) form of the toxin. By way of example, a modified clostridial neurotoxin may have modified amino acid sequences in one or more domains relative to the native (unmodified) clostridial neurotoxin sequence. Such modifications may modify functional aspects of the neurotoxin, for example biological activity or persistence. Thus, in one embodiment, the first neurotoxin and/or the second neurotoxin is a modified neurotoxin, or modified neurotoxin derivative.

A modified neurotoxin retains at least one of the functions of a neurotoxin, selected from the ability to bind to a low or high affinity neurotoxin receptor on a target cell, to translocate the endopeptidase portion of the neurotoxin (light chain) into the cell cytoplasm and to cleave a SNARE protein. Preferably, a modified neurotoxin retains at least two of these functions. More preferably a modified neurotoxin retains these three functions.

A modified neurotoxin may have one or more modifications in the amino acid sequence of the heavy chain (such as a modified H_{C} domain), wherein said modified heavy chain binds to target nerve cells with a higher or lower affinity than the native (unmodified) neurotoxin. Such modifications in the H_{C} domain can include modifying residues in the ganglioside binding site of the H_{C} domain or in the protein (SV2 or synaptotagmin) binding site that alter binding to the ganglioside receptor and/or the protein receptor of the target nerve cell. Examples of such modified neurotoxins are described in WO 2006/027207 and WO 2006/114308, both of which are hereby incorporated by reference in their entirety.

A modified neurotoxin may have one or more modifications in the amino acid sequence of the light chain, for example modifications in the substrate binding or catalytic domain which may alter or modify the SNARE protein specificity of the modified LC. Examples of such modified neurotoxins are described in WO 2010/120766 and US 2011/0318385, both of which are hereby incorporated by reference in their entirety.

A modified neurotoxin may comprise one or more modifications that increases or decreases the biological activity and/or the biological persistence of the modified neurotoxin. For example, a modified neurotoxin may comprise a leucine- or tyrosine-based motif, wherein said motif increases or decreases the biological activity and/or the biological persistence of the modified neurotoxin. Suitable leucine-based motifs include xDxxxLL, xExxxLL, xExxxIL, and xExxxLM (wherein x is any amino acid). Suitable tyrosine-based motifs include Y-x-x-Hy (wherein Hy is a hydrophobic amino acid). Examples of modified neurotoxins comprising leucine- and tyrosine-based motifs are described in WO 2002/08268, which is hereby incorporated by reference in its entirety.

In one embodiment, the first or second neurotoxin is a modified BoNT/A which has an amino acid sequence having at least 70%, preferably at least 75%, 80%, 85%, 90%, 95% or 99% sequence identity to SEQ ID NO: 1.

In one embodiment, the first or second neurotoxin is a modified BoNT/B which has an amino acid sequence having at least 70%, preferably at least 75%, 80%, 85%, 90%, 95% or 99% sequence identity to SEQ ID NO: 2.

In one embodiment, the first or second neurotoxin is a modified BoNT/C which has an amino acid sequence having at least 70%, preferably at least 75%, 80%, 85%, 90%, 95% or 99% sequence identity to SEQ ID NO: 3.

In one embodiment, the first or second neurotoxin is a modified BoNT/D which has an amino acid sequence having at least 70%, preferably at least 75%, 80%, 85%, 90%, 95% or 99% sequence identity to SEQ ID NO: 4.

In one embodiment, the first or second neurotoxin is a modified BoNT/E which has an amino acid sequence having at least 70%, preferably at least 75%, 80%, 85%, 90%, 95% or 99% sequence identity to SEQ ID NO: 5.

In one embodiment, the first or second neurotoxin is a modified BoNT/F which has an amino acid sequence having at least 70%, preferably at least 75%, 80%, 85%, 90%, 95% or 99% sequence identity to SEQ ID NO: 6.

In one embodiment, the first or second neurotoxin is a modified BoNT/G which has an amino acid sequence having at least 70%, preferably at least 75%, 80%, 85%, 90%, 95% or 99% sequence identity to SEQ ID NO: 7.

In one embodiment, the first or second neurotoxin is a modified TeNT which has an amino acid sequence having at least 70%, preferably at least 75%, 80%, 85%, 90%, 95% or 99% sequence identity to SEQ ID NO: 8.

In one embodiment, the second neurotoxin is a BoNT/B. Such a chimeric neurotoxin is referred to herein as a “BoNT/XB neurotoxin”.

In a preferred embodiment, the first neurotoxin is a BoNT/A and the second neurotoxin is a BoNT/B. Such a chimeric neurotoxin is referred to herein as a “BoNT/AB neurotoxin”. More preferably, the first neurotoxin is a BoNT/A1 and the second neurotoxin is a BoNT/B1. More preferably still, the LH_{N} domain from a first neurotoxin corresponds to amino acid residues 1 to 872 of BoNT/A1 and the H_{C} domain from a second neurotoxin corresponds to amino acid residues 860 to 1291 of BoNT/B1. In one preferred embodiment, the LH_{N} domain from a first neurotoxin corresponds to amino acid residues 1 to 872 of SEQ ID NO: 1 and the H_{C} domain from a second neurotoxin corresponds to amino acid residues 860 to 1291 of SEQ ID NO: 2.

In other words, a preferred chimeric neurotoxin according to the invention comprises or consists of the amino acid sequence SEQ ID NO:13.

Compared to the BoNT/A serotype, natural BoNT/B is much less potent despite a comparatively greater abundance of its receptor on synaptic vesicles. This is due to a unique amino acid change within the toxin binding site in human synaptotagmin II (Syt II) as compared to rodent (rat/mouse) Syt II (Peng, L., et al., J Cell Sci, 125(Pt 13):3233-42 (2012); Rummel, A. et al, FEBS J 278:4506-4515 (2011).13,22. As a result of this residue change, human Syt II has greatly diminished binding to natural BoNT/B, as well as to natural BoNT/D-C, and/G.

These findings provide an explanation for the clinical observations that a much higher dose of BoNT/B than BoNT/A (which binds a different receptor) is needed to achieve the same levels of therapeutic effects in patients. In a preferred embodiment of a BoNT/XB or BoNT/AB neurotoxin according to the invention, the H_{C} domain from a BoNT/B neurotoxin comprises at least one amino acid residue substitution, addition or deletion in the H_{CC} subdomain which has the effect of increasing the binding affinity of BoNT/B neurotoxin for human Syt II as compared to the natural BoNT/B sequence.

Suitable amino acid residue substitution, addition or deletion in the BoNT/B H_{CC} subdomain have been disclosed in WO2013/180799 and in PCT/US2016/024211 which is not yet published (both herein incorporated by reference).

Suitable amino acid residue substitution, addition or deletion in the BoNT/B H_{CC} subdomain include substitution mutations selected from the group consisting of: V1118M; Y1183M; E1191M; E11911; E1191Q; E1191T; 51199Y; 51199F; 51199L; 51201V; E1191C, E1191V, E1191L, E1191Y, S1199W, 51199E, 51199H, W1178Y, W1178Q, W1178A, W1178S, Y1183C, Y1183P and combinations thereof.

Suitable amino acid residue substitution, addition or deletion in the BoNT/B H_{CC} subdomain further include combinations of two substitution mutations selected from the group consisting of: E1191M and 51199L, E1191M and 51199Y, E1191M and 51199F, E1191Q and 51199L, E1191Q and 51199Y, E1191Q and 51199F, E1191M and S1199W, E1191M and W1178Q, E1191C and S1199W, E1191C and 51199Y, E1191C and W1178Q, E1191Q and S1199W, E1191V and S1199W, E1191V and S1199Y, or E1191V and W1178Q.

Suitable amino acid residue substitution, addition or deletion in the BoNT/B H_{CC} subdomain also include a combination of three substitution mutations which are E1191M, S1199W and W1178Q.

In a preferred embodiment, the suitable amino acid residue substitution, addition or deletion in the BoNT/B H_{CC} subdomain include a combination of two substitution mutations which are E1191M and S1199Y. In other words, a preferred chimeric neurotoxin according to the invention comprises or consists of the amino acid sequence SEQ ID NO: 11 or SEQ ID NO: 12.

In another preferred embodiment, the first neurotoxin is a BoNT/C and the second neurotoxin is a BoNT/B. Such a chimeric neurotoxin is referred to herein as a “BoNT/CB neurotoxin”. More preferably, the first neurotoxin is a BoNT/C1 and the second neurotoxin is a BoNT/B1. More preferably still, the LH_{N} domain from a first neurotoxin corresponds to amino acid residues 1 to 867 of BoNT/C1 and the H_{C} domain from a second neurotoxin corresponds to amino acid residues 860 to 1291 of BoNT/B1. In one preferred embodiment, the LH_{N} domain from a first neurotoxin corresponds to amino acid residues 1 to 867 of SEQ ID NO: 3 and the H_{C} domain from a second neurotoxin corresponds to amino acid residues 860 to 1291 of SEQ ID NO: 2. In a preferred embodiment, the He domain from the BoNT/B neurotoxin comprises at least one amino acid residue substitution, addition or deletion in the H_{CC} subdomain which has the effect of increasing the binding affinity of BoNT/B neurotoxin for human Syt II as compared to the natural BoNT/B sequence. Suitable amino acid residue substitution, addition or deletion in the BoNT/B H_{CC} subdomain is as described above.

In a preferred embodiment of a BoNT/XDC neurotoxin (chimeric neurotoxin in which the second neurotoxin is a mosaic BoNT/DC) according to the invention, the H_{C} domain from a mosaic BoNT/DC neurotoxin comprises at least one amino acid residue substitution, addition or deletion in the H_{CC} subdomain which has the effect of increasing the binding affinity of mosaic BoNT/DC neurotoxin for human Syt II as compared to the natural mosaic BoNT/DC sequence.

In a preferred embodiment of a BoNT/XG neurotoxin according to the invention, the H_{C} domain from a BoNT/G neurotoxin comprises at least one amino acid residue substitution, addition or deletion in the H_{CC} subdomain which has the effect of increasing the binding affinity of BoNT/G neurotoxin for human Syt II as compared to the natural BoNT/G sequence.

Other preferred neurotoxins according to the invention are as follows.

In a preferred embodiment, the first neurotoxin is a BoNT/A and the second neurotoxin is a BoNT/C. Such a chimeric neurotoxin is referred to herein as a “BoNT/AC neurotoxin”. More preferably, the first neurotoxin is a BoNT/A1 and the second neurotoxin is a BoNT/C1. More preferably still, the LH_{N} domain from a first neurotoxin corresponds to amino acid residues 1 to 872 of BoNT/A1 and the H_{C} domain from a second neurotoxin corresponds to amino acid residues 868 to 1291 of BoNT/C1. In one preferred embodiment, the LH_{N} domain from a first neurotoxin corresponds to amino acid residues 1 to 872 of SEQ ID NO: 1 and the H_{C} domain from a second neurotoxin corresponds to amino acid residues 868 to 1291 of SEQ ID NO: 3.

In another preferred embodiment, the first neurotoxin is a BoNT/B and the second neurotoxin is a BoNT/A. Such a chimeric neurotoxin is referred to herein as a “BoNT/BA neurotoxin”. More preferably, the first neurotoxin is a BoNT/B1 and the second neurotoxin is a BoNT/A1. More preferably still, the LH_{N} domain from a first neurotoxin corresponds to amino acid residues 1 to 859 of BoNT/B1 and the H_{C} domain from a second neurotoxin corresponds to amino acid residues 873 to 1296 of BoNT/A1. In one preferred embodiment, the LH_{N} domain from a first neurotoxin corresponds to amino acid residues 1 to 859 of SEQ ID NO: 2 and the H_{C} domain from a second neurotoxin corresponds to amino acid residues 873 to 1293 of SEQ ID NO: 1.

In another preferred embodiment, the first neurotoxin is a BoNT/B and the second neurotoxin is a BoNT/C. Such a chimeric neurotoxin is referred to herein as a “BoNT/BC neurotoxin”. More preferably, the first neurotoxin is a BoNT/B1 and the second neurotoxin is a BoNT/C1. More preferably still, the LH_{N} domain from a first neurotoxin corresponds to amino acid residues 1 to 859 of BoNT/B1 and the H_{C} domain from a second neurotoxin corresponds to amino acid residues 868 to 1291 of BoNT/C1. In one preferred embodiment, the LH_{N} domain from a first neurotoxin corresponds to amino acid residues 1 to 859 of SEQ ID NO: 2 and the H_{C} domain from a second neurotoxin corresponds to amino acid residues 868 to 1291 of SEQ ID NO: 3. In other words, a preferred chimeric neurotoxin according to the invention comprises or consists of the amino acid sequence SEQ ID NO: 56.

In another preferred embodiment, the first neurotoxin is a BoNT/C and the second neurotoxin is a BoNT/A. Such a chimeric neurotoxin is referred to herein as a “BoNT/CA neurotoxin”. More preferably, the first neurotoxin is a BoNT/C1 and the second neurotoxin is a BoNT/A1. More preferably still, the LH_{N} domain from a first neurotoxin corresponds to amino acid residues 1 to 867 of BoNT/C1 and the H_{C} domain from a second neurotoxin corresponds to amino acid residues 873 to 1296 of BoNT/A1. In one preferred embodiment, the LH_{N} domain from a first neurotoxin corresponds to amino acid residues 1 to 867 of SEQ ID NO: 3 and the H_{C} domain from a second neurotoxin corresponds to amino acid residues 873 to 1296 of SEQ ID NO: 1.

The chimeric neurotoxins of the present invention can be produced using recombinant technologies. Thus, in one embodiment, a chimeric neurotoxin according to the invention is a recombinant chimeric neurotoxin. It shall be readily understood that, according to this preferred embodiment, a nucleotide sequence encoding a recombinant chimeric neurotoxin of the invention, a vector comprising said nucleotide sequence, and a cell comprising said vector, as further described below, can mutatis mutandis be referred as being recombinant.

In another aspect, the invention provides a nucleotide sequence encoding a chimeric neurotoxin according to the invention, for example a DNA or RNA sequence. In a preferred embodiment, the nucleotide sequence is a DNA sequence.

The nucleic acid molecules of the invention may be made using any suitable process known in the art. Thus, the nucleic acid molecules may be made using chemical synthesis techniques.

Alternatively, the nucleic acid molecules of the invention may be made using molecular biology techniques.

The DNA sequence of the present invention is preferably designed in silico, and then synthesised by conventional DNA synthesis techniques.

The above-mentioned nucleic acid sequence information is optionally modified for codon-biasing according to the ultimate host cell (e.g. E. coli) expression system that is to be employed.

In another aspect, the invention provides a vector comprising a nucleotide sequence according to the invention. In one embodiment, the nucleic acid sequence is prepared as part of a DNA vector comprising a promoter and terminator. In a preferred embodiment, the vector has a promoter selected from Tac, AraBAD, T7-Lac, or T5-Lac.

A vector may be suitable for in vitro and/or in vivo expression of the above-mentioned nucleic acid sequence. The vector can be a vector for transient and/or stable gene expression. The vector may additionally comprise regulatory elements and/or selection markers. Said vector may be of viral origin, of phage origin, or of bacterial origin. For example, said expression vector may be a pET, pJ401, pGEX vector or a derivative thereof.

In another aspect, the invention provides a cell comprising a nucleotide sequence or a vector according to the invention. The term “cell” can herein be used interchangeably with the term “host cell” or “cell line”. Suitable cell type includes prokaryotic cells, for example E. coli, and eukaryotic cells, such as yeast cells, mammalian cells, insect cells, etc. Preferably, the cell is E. coli.

In another aspect, the invention provides a method for producing a chimeric neurotoxin according to the invention, said method comprising the step of culturing a cell as described above, under conditions wherein said chimeric neurotoxin is produced. Said conditions are well-known to the skilled practitioner and therefore need not be further detailed herein.

Preferably, said method further comprises the step of recovering the chimeric neurotoxin from the culture.

In another aspect, the invention provides a pharmaceutical composition comprising a chimeric neurotoxin according to the invention. Preferably, the pharmaceutical composition comprises a chimeric neurotoxin together with at least one component selected from a pharmaceutically acceptable carrier, excipient, adjuvant, propellant and/or salt.

In another aspect, the invention provides a chimeric neurotoxin or pharmaceutical composition according to the invention for use in therapy. More precisely, the invention relates to the use of a chimeric neurotoxin or pharmaceutical composition as described herein, for manufacturing a medicament. In other words, the invention relates to a method for treating a subject in need thereof, comprising the step of administering an effective amount of a chimeric neurotoxin or pharmaceutical composition as described herein, to said subject. By “effective amount”, it is meant that the chimeric neurotoxin or pharmaceutical composition is administered in a quantity sufficient to provide the effect for which it is indicated. As used herein, the term “subject” preferably refers to a human being or an animal, more preferably to a human being.

A chimeric neurotoxin according to the invention is preferably suitable for use in treating a condition associated with unwanted neuronal activity in a subject in need thereof, for example a condition selected from the group consisting of spasmodic dysphonia, spasmodic torticollis, laryngeal dystonia, oromandibular dysphonia, lingual dystonia, cervical dystonia, focal hand dystonia, blepharospasm, strabismus, hemifacial spasm, eyelid disorder, cerebral palsy, focal spasticity and other voice disorders, spasmodic colitis, neurogenic bladder, anismus, limb spasticity, tics, tremors, bruxism, anal fissure, achalasia, dysphagia and other muscle tone disorders and other disorders characterized by involuntary movements of muscle groups, lacrimation, hyperhidrosis, excessive salivation, excessive gastrointestinal secretions, secretory disorders, pain from muscle spasms, headache pain, migraine and dermatological conditions. More precisely, the invention relates to the use of a chimeric neurotoxin or pharmaceutical composition as described herein, for manufacturing a medicament intended to treat a condition associated with unwanted neuronal activity, as described above. In other words, the invention relates to a method for treating a condition associated with unwanted neuronal activity, as described above, in a subject in need thereof, said method comprising the step of administering an effective amount of a chimeric neurotoxin or pharmaceutical composition as described herein, to said subject.

In another aspect, the invention provides a non-therapeutic use of a chimeric neurotoxin according to the invention for treating an aesthetic or cosmetic condition, in a subject in need thereof. In other words, the invention relates to a method for treating an aesthetic or cosmetic condition in a subject in need thereof, comprising the step of administering an effective amount of a chimeric neurotoxin or pharmaceutical composition as described herein, to said subject.

According to this aspect of the invention, the subject to be treated is preferably not suffering from a condition associated with unwanted neuronal activity as described above. More preferably, said subject is a healthy subject, i.e. a subject which is not suffering from any disease.

In another aspect, the invention provides a kit for use in a therapeutic or non-therapeutic (cosmetic or aesthetic) method, or for a therapeutic or non-therapeutic (cosmetic or aesthetic) use, as described above, said kit comprising a pharmaceutical composition of the invention and instructions for performing said method or use. More precisely, the invention relates to a kit comprising a pharmaceutical composition of the invention and instructions for therapeutic or cosmetic administration of said composition to a subject in need thereof. As used herein, the term “instructions” refers to a publication, a recording, a diagram, or any other medium of expression which can be used to communicate how to perform a method or use of the invention, such as therapeutic or cosmetic administration of said composition to a subject in need thereof. Said instructions can, for example, be affixed to a container which comprises said composition or said kit.

The engineered chimeric neurotoxins of the present invention may be formulated for oral, parenteral, continuous infusion, inhalation or topical application. Compositions suitable for injection may be in the form of solutions, suspensions or emulsions, or dry powders which are dissolved or suspended in a suitable vehicle prior to use.

In the case of a chimeric neurotoxin that is to be delivered locally, the chimeric neurotoxin may be formulated as a cream (e.g. for topical application), or for sub-dermal injection.

Local delivery means may include an aerosol, or other spray (e.g. a nebuliser). In this regard, an aerosol formulation of a chimeric neurotoxin enables delivery to the lungs and/or other nasal and/or bronchial or airway passages.

Chimeric neurotoxins of the invention may be administered to a patient by intrathecal or epidural injection in the spinal column at the level of the spinal segment involved in the innervation of an affected organ.

A preferred route of administration is via laparoscopic and/or localised, particularly intramuscular, injection.

The dosage ranges for administration of the chimeric neurotoxins of the present invention are those to produce the desired therapeutic effect. It will be appreciated that the dosage range required depends on the precise nature of the chimeric neurotoxin or composition, the route of administration, the nature of the formulation, the age of the patient, the nature, extent or severity of the patient's condition, contraindications, if any, and the judgement of the attending physician. Variations in these dosage levels can be adjusted using standard empirical routines for optimisation.

Fluid dosage forms are typically prepared utilising the chimeric neurotoxin and a pyrogen-free sterile vehicle. The engineered clostridial toxin, depending on the vehicle and concentration used, can be either dissolved or suspended in the vehicle. In preparing solutions the chimeric neurotoxin can be dissolved in the vehicle, the solution being made isotonic if necessary by addition of sodium chloride and sterilised by filtration through a sterile filter using aseptic techniques before filling into suitable sterile vials or ampoules and sealing. Alternatively, if solution stability is adequate, the solution in its sealed containers may be sterilised by autoclaving. Advantageously additives such as buffering, solubilising, stabilising, preservative or bactericidal, suspending or emulsifying agents and or local anaesthetic agents may be dissolved in the vehicle.

Dry powders, which are dissolved or suspended in a suitable vehicle prior to use, may be prepared by filling pre-sterilised ingredients into a sterile container using aseptic technique in a sterile area. Alternatively, the ingredients may be dissolved into suitable containers using aseptic technique in a sterile area. The product is then freeze dried and the containers are sealed aseptically.

Parenteral suspensions, suitable for intramuscular, subcutaneous or intradermal injection, are prepared in substantially the same manner, except that the sterile components are suspended in the sterile vehicle, instead of being dissolved and sterilisation cannot be accomplished by filtration. The components may be isolated in a sterile state or alternatively it may be sterilised after isolation, e.g. by gamma irradiation.

Administration in accordance with the present invention may take advantage of a variety of delivery technologies including microparticle encapsulation, viral delivery systems or high-pressure aerosol impingement.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a clostridial neurotoxin” includes a plurality of such candidate agents and reference to “the clostridial neurotoxin” includes reference to one or more clostridial neurotoxins and equivalents thereof known to those skilled in the art, and so forth.

The invention will now be described, by way of example only, with reference to the following Figures and Examples.

### DESCRIPTION OF THE DRAWINGS

FIGS. 1A-X—Sequence alignment of BoNT/A1-8, /B1-8, /C, /D, /E1-12, /F1-7, /G, /“H”, and TeNT using CLUSTAL Omega (1.2.1) multiple sequence alignment tool. The location of the putative 3₁₀ helix separating the LH_{N} and H_{C} domains is in bold and underlined characters.

FIGS. 2A-C—SDS PAGE of purified recombinant BoNT/AB chimera 1 (FIG. 2A), 2 (FIG. 2B) and 3A (FIG. 2C) (SEQ ID NO: 9, 10 and 11 respectively). Lanes are labelled “Marker” (molecular weight marker), “-DTT” (oxidised BoNT/AB chimera sample), and “+DTT” (reduced BoNT/AB chimera sample).

FIG. 3—Cleavage of SNAP-25 in rat spinal cord neurons by recombinant BoNT/AB chimera 1, 2 and 3A (SEQ ID NOs: 9, 10 and 11 respectively). Cultured rat primary spinal cord neurons (SCN) were exposed to various concentrations of recombinant BoNT/AB chimera 1, 2 or 3A for 24 hours, at 37° C. in a humidified atmosphere with 10% CO₂. Cells were then lysed with 1×NUPAGE™ buffer supplemented with DTT and BENZONASE® nuclease. The samples were transferred to microcentrifuge tubes, heated for 5 min at 90° C. on heat block and stored at −20° C. before analysis of SNAP-25 cleavage by Western blot. SNAP-25 was detected using a polyclonal antibody, that detects both the full length and cleaved forms of SNAP-25 (Sigma #S9684). Anti-rabbit HRP (Sigma #A6154) was used as the secondary antibody.

FIG. 4—Mouse digit abduction scoring assay. Mice were injected into the gastrocnemius-soleus complex muscles of one hind limb, under short general anaesthesia; muscle weakening was measured on a 0-4 scale using the digit abduction score (DAS). DAS max values were determined for each dose and plotted against dose and the data were fitted to a 4-parameter logistic equation, ED50 and dose leading to DAS 4 (DAS 4 dose) values were determined.

FIGS. 5A and B—SDS PAGE of purified recombinant BoNT/AB chimera 3B (FIG. 5A) and 3C (FIG. 5B) (SEQ ID NO: 12 and 13 respectively). Lanes are labelled “Marker” (molecular weight marker), “-DTT” (oxidised BoNT/AB chimera sample), and “+DTT” (reduced BoNT/AB chimera sample).

FIG. 6—Cleavage of SNAP-25 by recombinant BoNT/A and BoNT/AB chimera 3B and 3C (SEQ ID NOs: 1, 12 and 13 respectively) in human induced pluripotent stem cell derived peripheral neurons (PERI.4U—Axiogenesis, Germany). PERI.4U cells were exposed to various concentrations of recombinant BoNT/A, or BoNT/AB chimera 3B or 3C for 24 hours, at 37° C. in a humidified CO₂ atmosphere containing 5% CO₂. Cells were then lysed with Ix NUPAGE™ buffer supplemented with DTT and BENZDNASE® nuclease. The samples were transferred to microcentrifuge tubes, heated for 5 min at 90° C. on heat block and stored at −20° C., before analysis of SNAP-25 cleavage by Western blot. SNAP-25 was detected using a polyclonal antibody, that detects both the full length and cleaved forms of SNAP-25 (Sigma #S9684). Anti-rabbit HRP (Sigma #A6154) was used as the secondary antibody.

FIG. 7—Duration of muscle weakening over time in the mouse digit abduction scoring assay. Mice were injected into the gastrocnemius-soleus complex muscles of one hind limb, under short general anaesthesia; muscle weakening was measured on a 0-4 scale using the digit abduction score (DAS). Animals of the group injected with the lowest dose that induced during the first four days of injection a DAS of 4 were monitored until complete recovery of the muscle weakness to a DAS of 0 (no observed muscle weakness).

FIG. 8—SDS PAGE of purified recombinant BoNT/BC (SEQ ID NO: 56). Lanes are labelled “Marker” (molecular weight marker), “-DTT” (oxidised BoNT/BC chimera sample), and “+DTT” (reduced BoNT/BC chimera sample).

FIG. 9—Cleavage of VAMP-2 by native BoNT/B. BoNT/BC chimera, and inactive recombinant BoNT/B (SEQ II) NOs: 2, 56 and 57, respectively) in rat cortical neurons. Cells were exposed to various concentrations of BoNT for 24 hours, at 37° C. in a humidified CO₂ atmosphere containing 5% CO₂. Cells were lysed with 1×NUPAGE™ buffer supplemented with DTT and BENZONASE® nuclease, and heated for 5 min at 90° C. before storage at −20° C. Samples were analysed for VAMP-2 cleavage by Western blot using a polyclonal rabbit anti-VAMP-2 (Abeam ab3347, 1:1000) primary antibody, and an HRP-conjugated anti-rabbit secondary antibody (Sigma #A6154).

FIG. 10—Cleavage of VAMP-2 peptide reporter by native BoNT/B and BoNT/BC chimera (SEQ ID NO: 2 and 56, respectively) using the BOTEST® BoNT detection kit (BioSentinel). Various concentrations of BoNT were incubated at 30° C. for 18 hours with a VAMP-2 peptide with a CFP-YFP FRET pair as a reporter and the proportion of uncleaved:cleaved reporter substrate was measured as a loss of YFP fluorescence intensity at 528 nm and gain of CFP fluorescence at 485 nm following excitation at 440 nm.

### EXAMPLES

The following Examples serve to illustrate particular embodiments of the invention, and do not limit the scope of the invention defined in the claims in any way.

### Example 1—Mapping of 3₁₀ Helix in Clostridial Neurotoxins

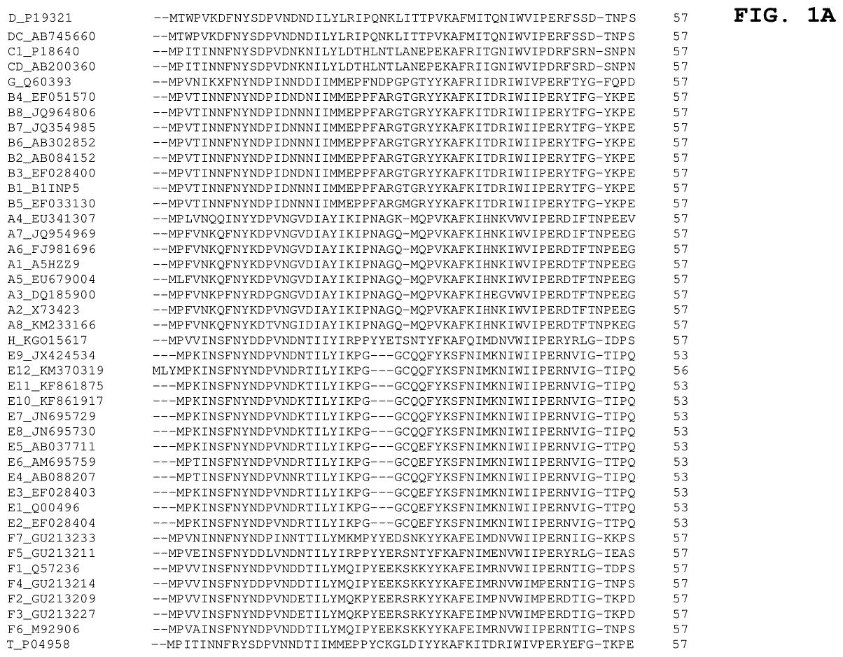

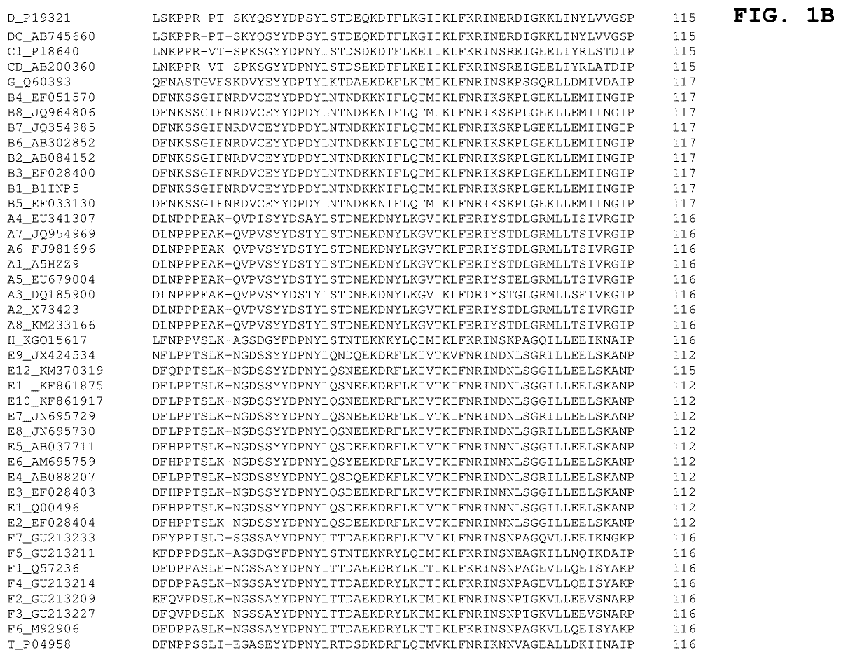

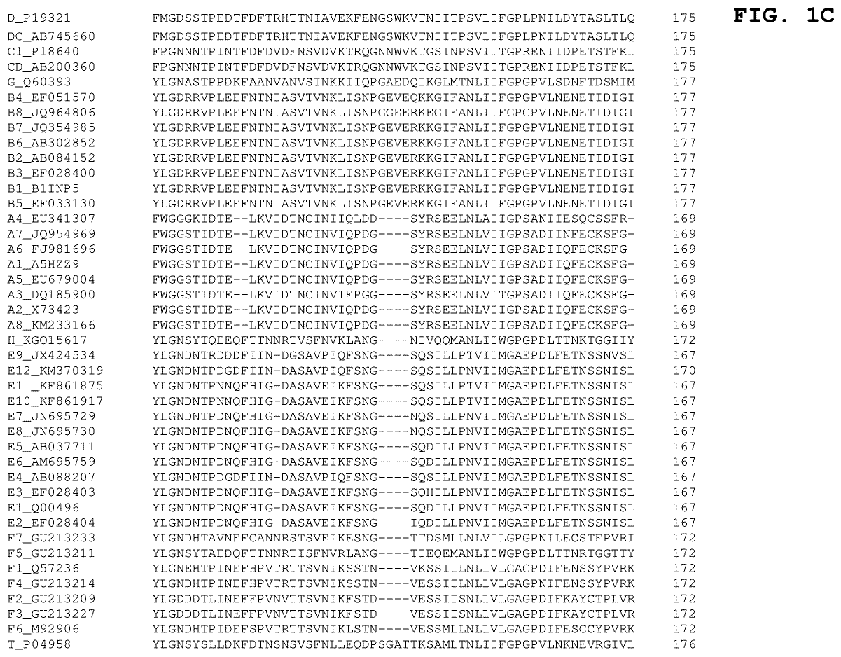

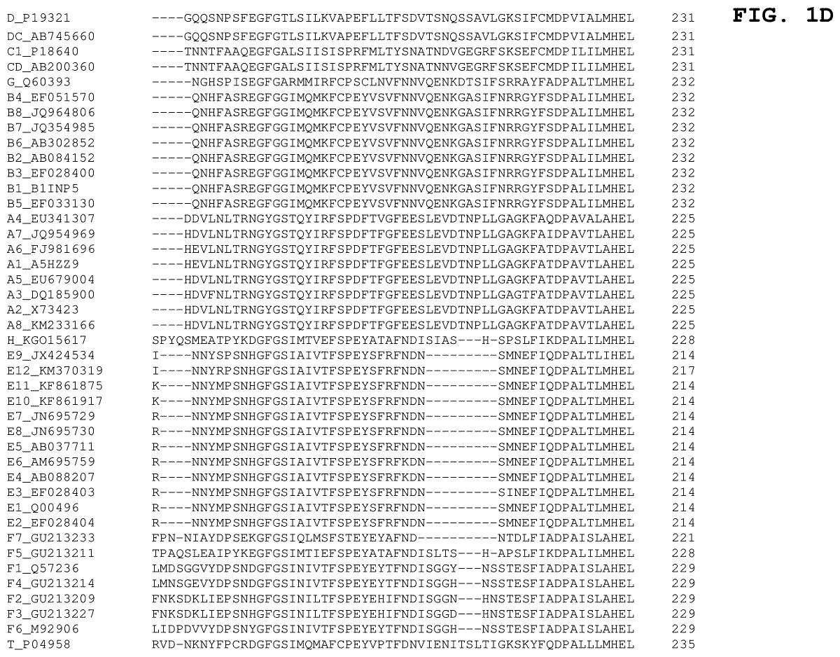

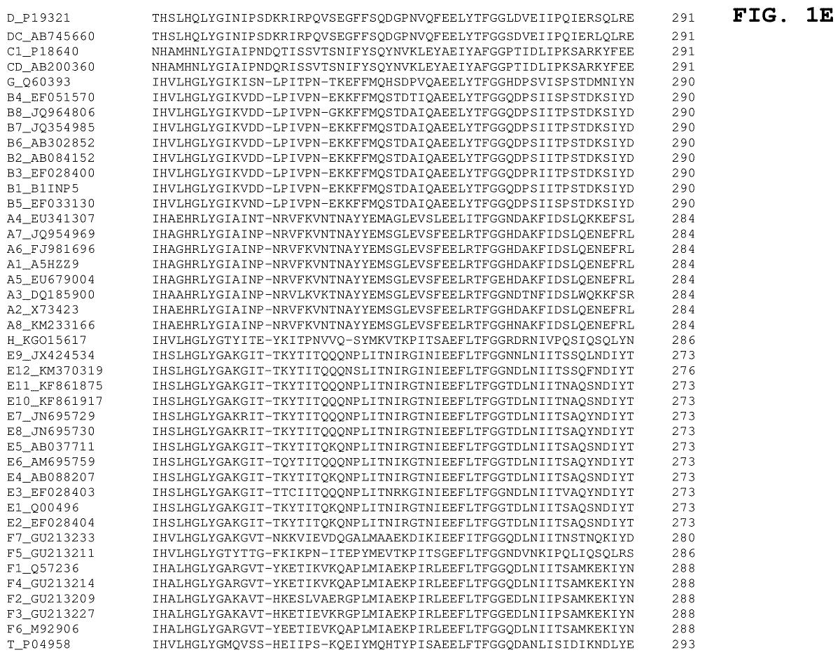

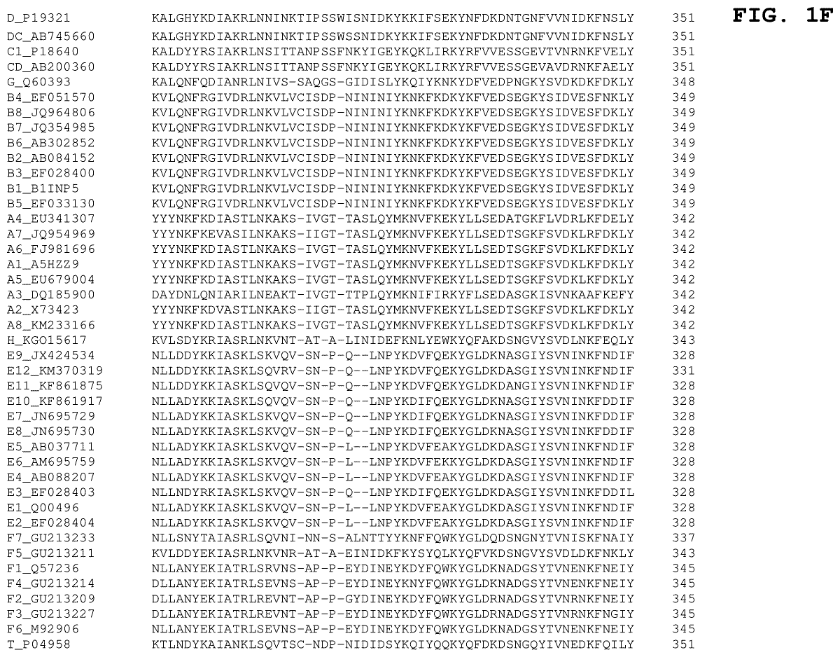

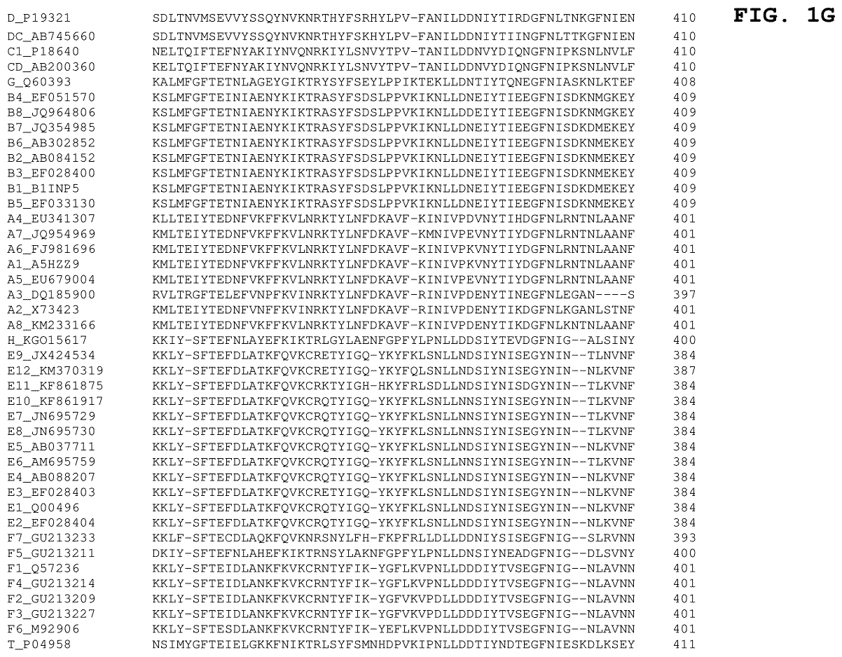

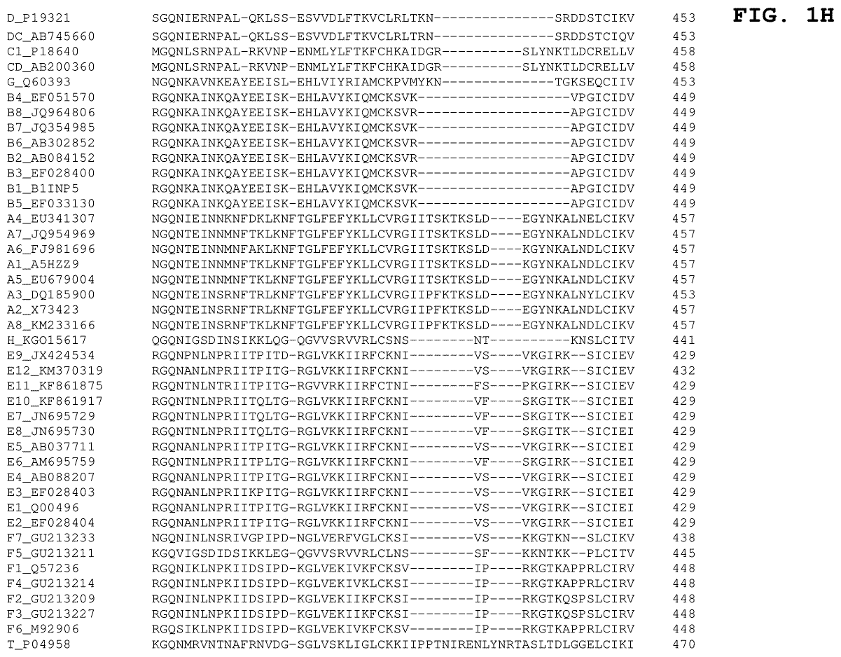

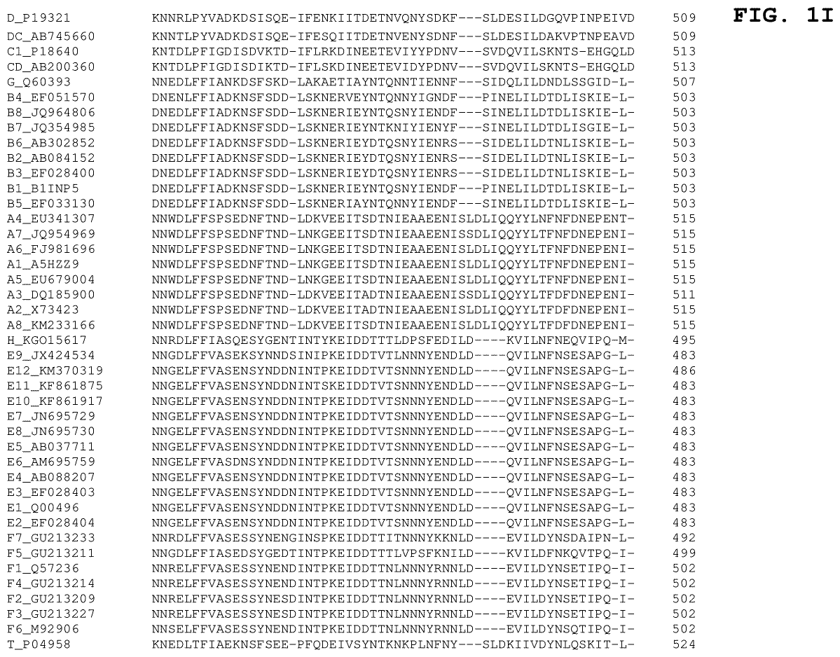

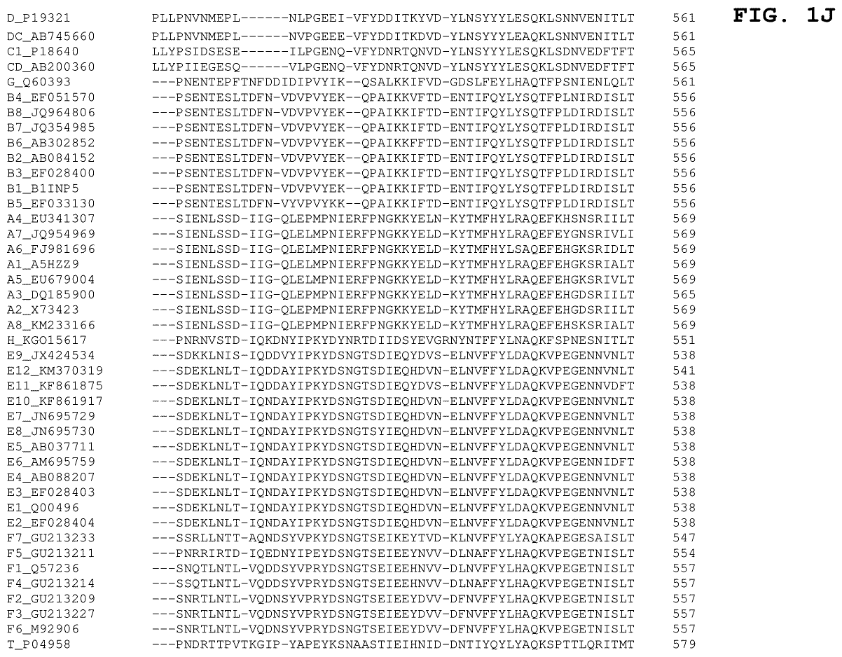

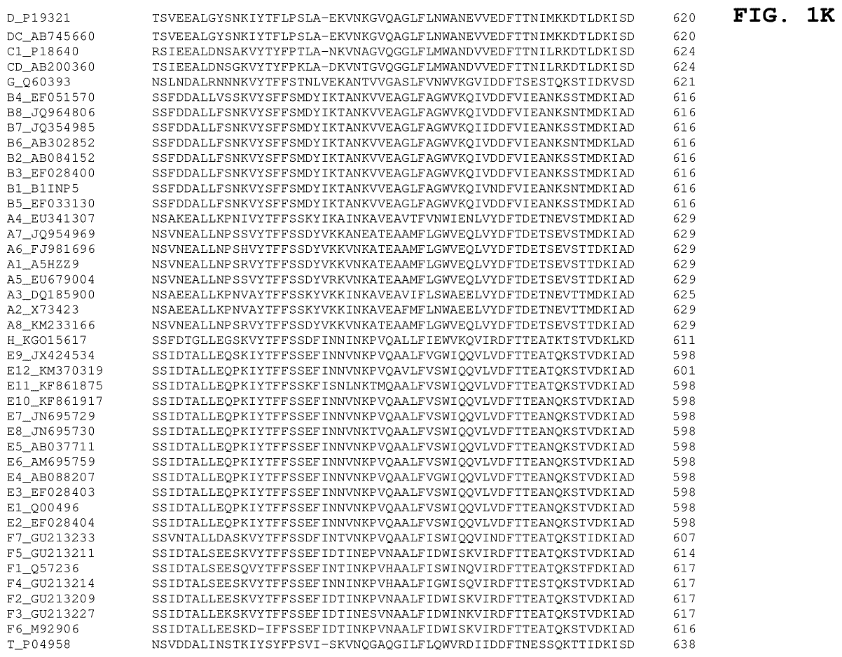

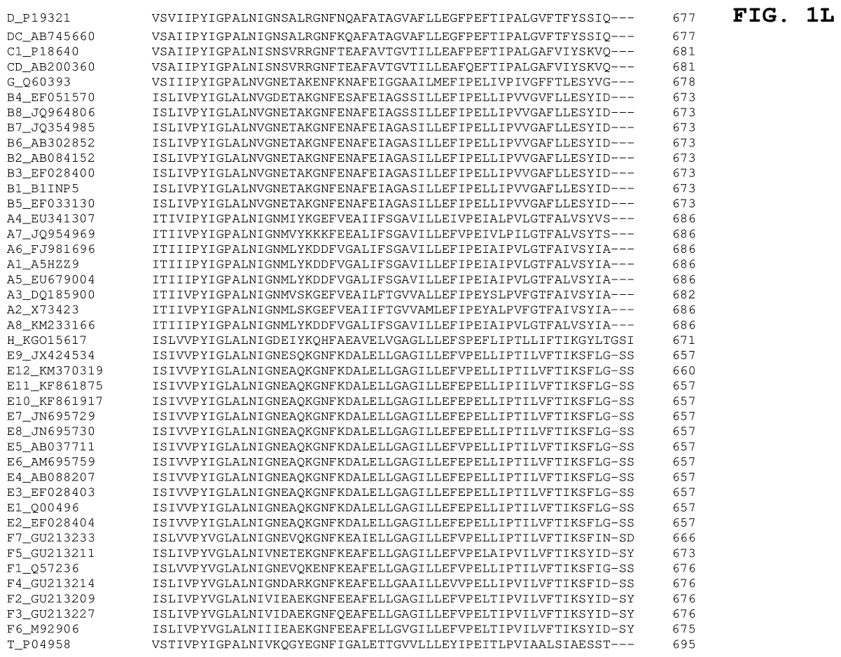

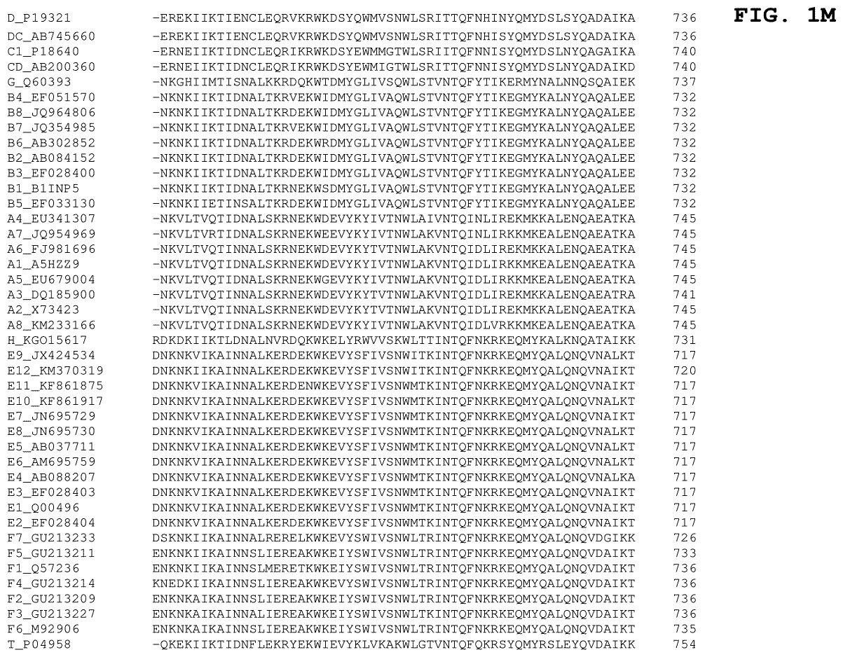

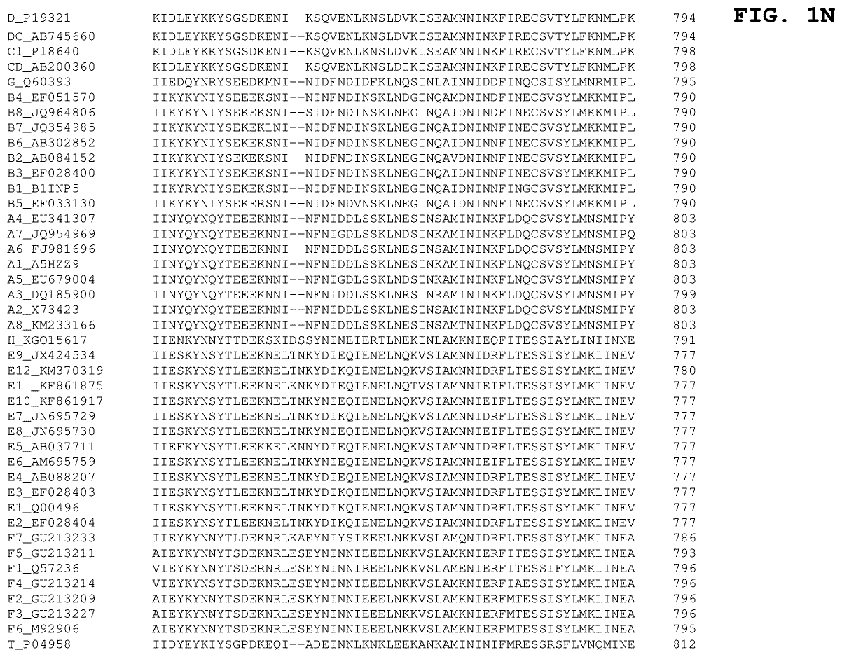

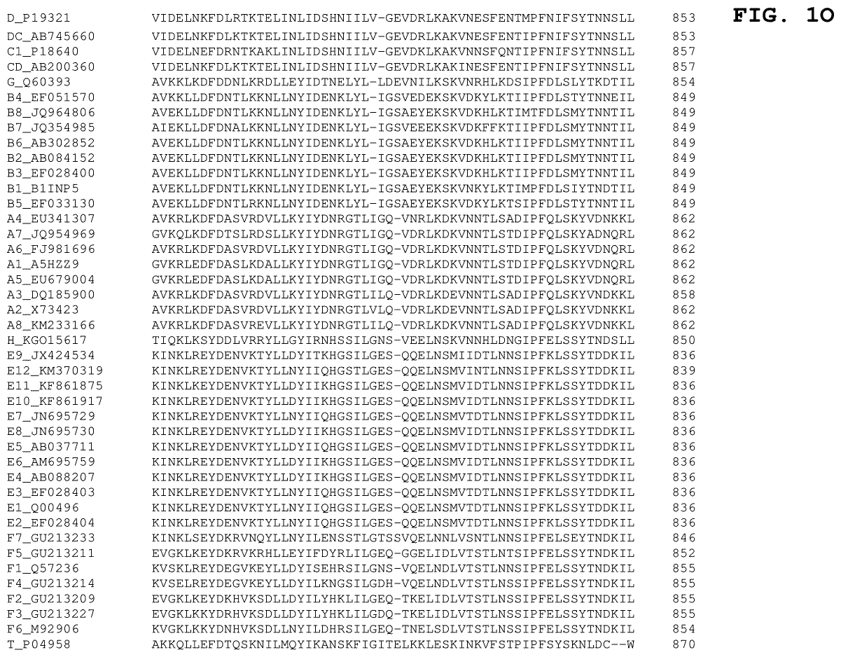

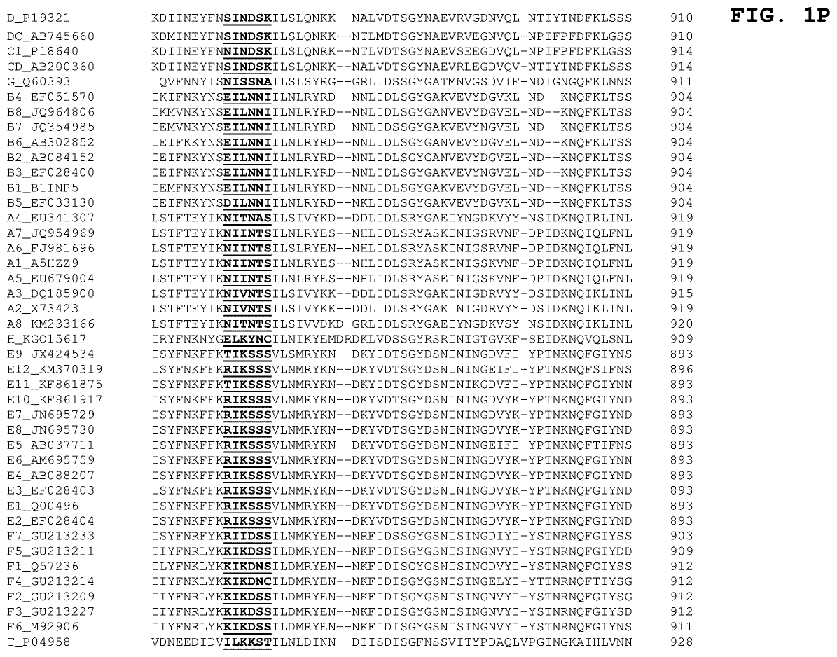

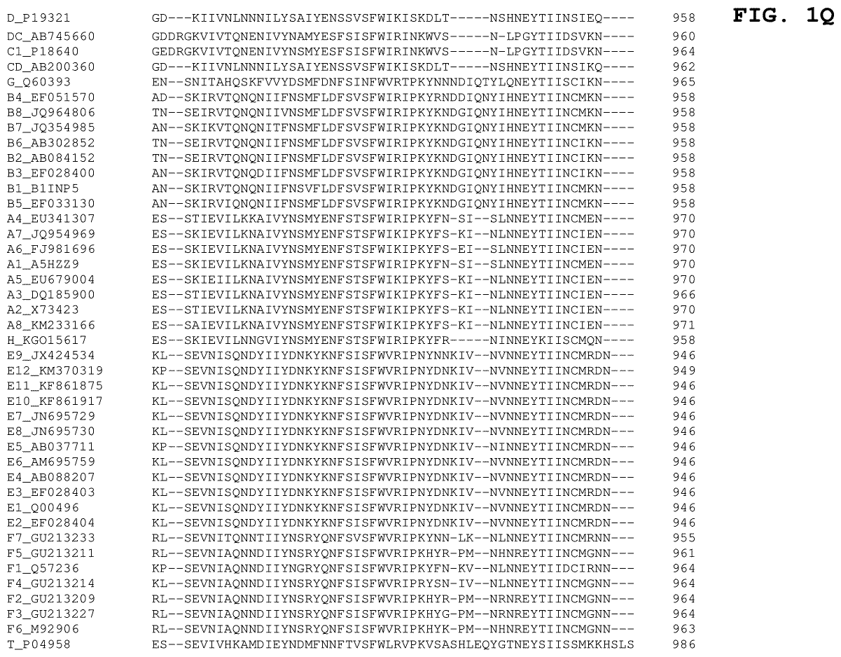

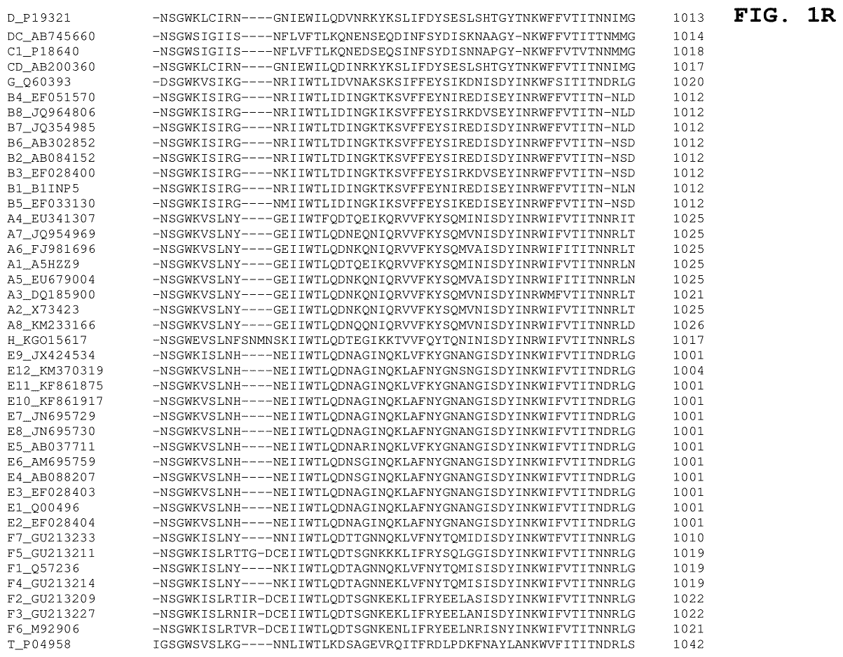

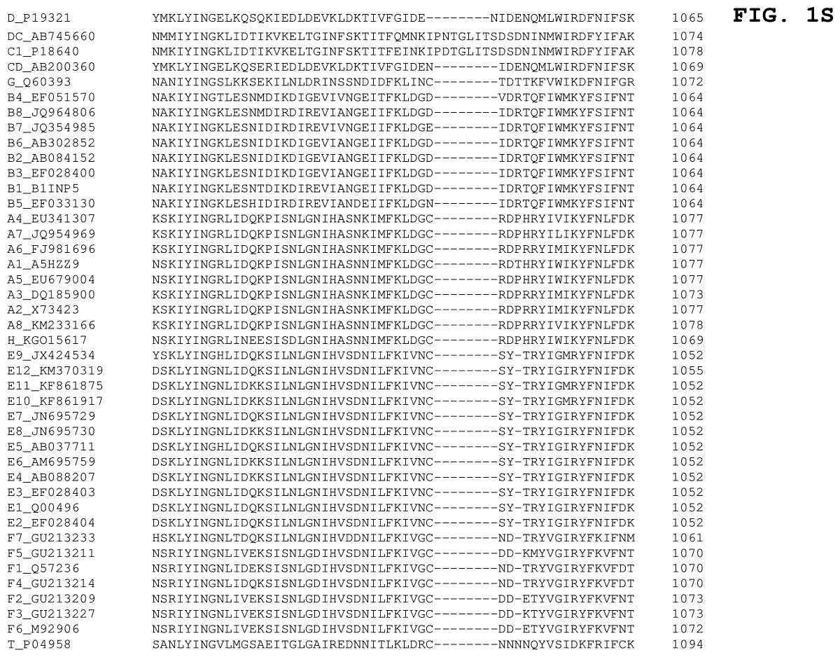

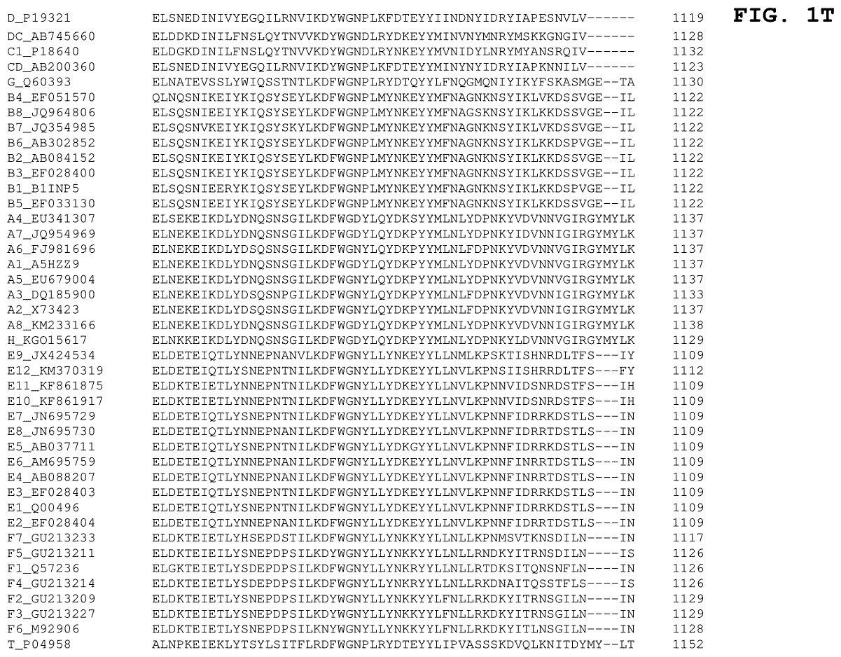

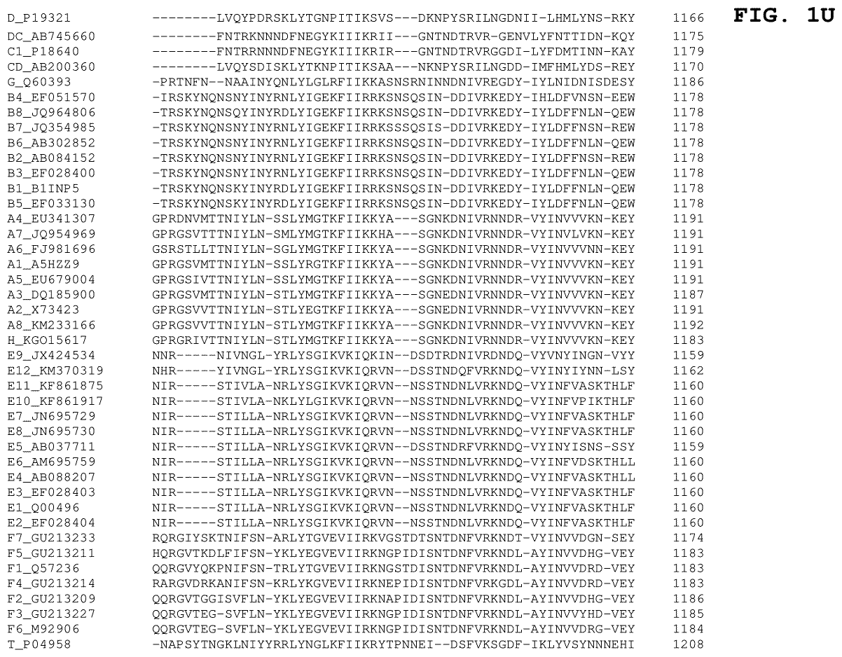

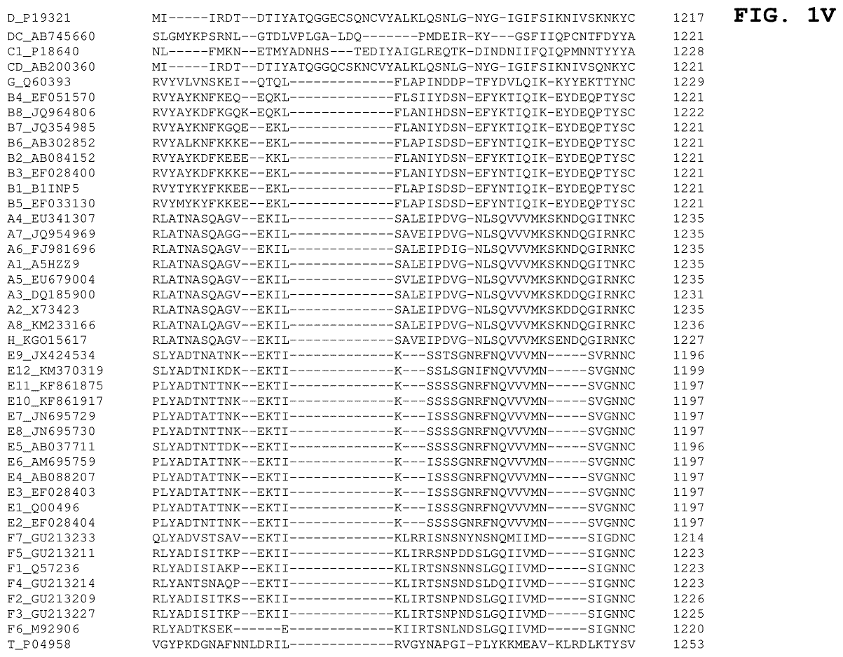

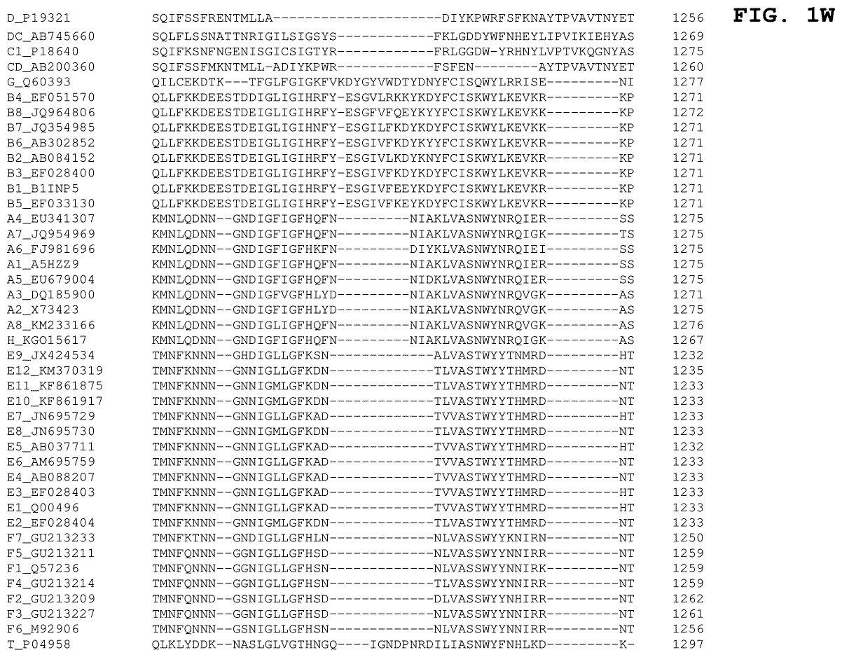

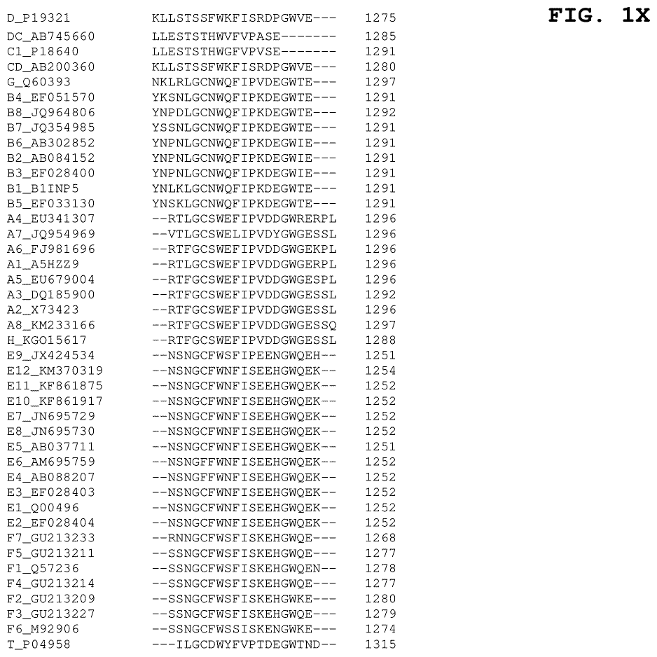

The amino acid sequences of all BoNT serotypes and TeNT were obtained from a public database (e.g., UNIPROT® or the National Center for Biotechnology Information) NCBI and then modelled onto the known crystal structure of BoNT/A1 (3BTA.pdb) using LOOPP. This yielded a predicted protein structure which was edited to retain only to N-terminal part of the HCN domain and ˜80 residues before it (C-terminal part of the H_{N} domain)—this domain (“H_{N}/H_{CN}”) is structurally highly conserved, therefore, making it the best region to superimpose different serotypes. Each edited structure was then superimposed onto 3BTA.pdb using SuperPose and the residues corresponding to a conspicuous 3₁₀ helix at the start of the Hc of BoNT/A1 (⁸⁷²NIINTS⁸⁷⁶) and corresponding residues in the other serotype were then identified. These were cross-checked with a sequence alignment of all BoNT serotypes with Clustal Omega (FIG. 1).

By identifying this region of structural equivalence between different neurotoxins, it was possible to identify a specific point at which a C-terminal half of one neurotoxin may transition over to a N-terminal half of another neurotoxin without interrupting the secondary structure of the overall molecule. This point was chosen to be the start of the 3₁₀ helix.

The results are presented in table 2 above.

### Example 2—Cloning, Expression and Purification of BoNT/AB Chimeras

BoNT/AB chimeric constructs 1, 2, 3A, 3B, and 3C (SEQ ID NO: 9 to 13) were constructed from DNA encoding the parent serotype molecule and appropriate oligonucleotides using standard molecular biology techniques. These were then cloned into the pJ401 expression vector with or without a C-terminal His₁₀-tag and transformed into BLR (DE3) E. coli cells for over-expression. These cells were grown at 37° C. and 225 RPM shaking in 2 L baffled conical flasks containing 1 L modified Terrific Broth (mTB) supplemented with the appropriate antibiotic. Once the A₆₀₀ reached >0.5, the incubator temperature was decreased to 16° C., and then induced with 1 mM IPTG an hour later for 20 h at 225 RPM shaking, to express the recombinant BoNT/AB construct.

Harvested cells were lysed by ultrasonication and clarified by centrifugation at 4500 RPM for 1 h at 4° C. The recombinant BoNT/AB chimeric molecules were then extracted in ammonium sulphate and purified by standard fast protein liquid chromatography (FPLC) techniques. This involved using a hydrophobic interaction resin for capture and an anion-exchange resin for the intermediate purification step. The partially purified molecules were then proteolytically cleaved with endoproteinase Lys-C to yield the active di-chain. This was further purified with a second hydrophobic interaction resin to obtain the final BoNT/AB chimera.

For BoNT/AB chimeric molecules with a decahistadine tag (H₁₀) (chimera 1, 2, 3A), the capture step employed the use of an immobilised nickel resin instead of the hydrophobic interaction resin.

The sequence of each chimera is presented in table 3.

TABLE 3
chimeric BoNT/AB constructs

| Molecule | SEQ ID NO | Sequence |
|---|---|---|
| Chimera 1 | 9 | A1:1-871 + B1:858-1291 |
| | | (E1191M/S1199Y) + His₁₀-tag |
| Chimera 2 | 10 | A1:1-874 + ELGGGGSEL + B1:858-1291 |
| | | (E1191M/S1199Y) + His₁₀-tag |
| Chimera 3A | 11 | A1:1-872 + B1:860-1291 |
| | | (E1191M/S1199Y) + His₁₀-tag |
| Chimera 3B | 12 | A1:1-872 + B1:860-1291 (E1191M/S1199Y) |
| Chimera 3C | 13 | A1:1-872 + B1:860-1291 |

### Example 3—Comparison of BoNT/AB Chimera 1, 2 and 3A

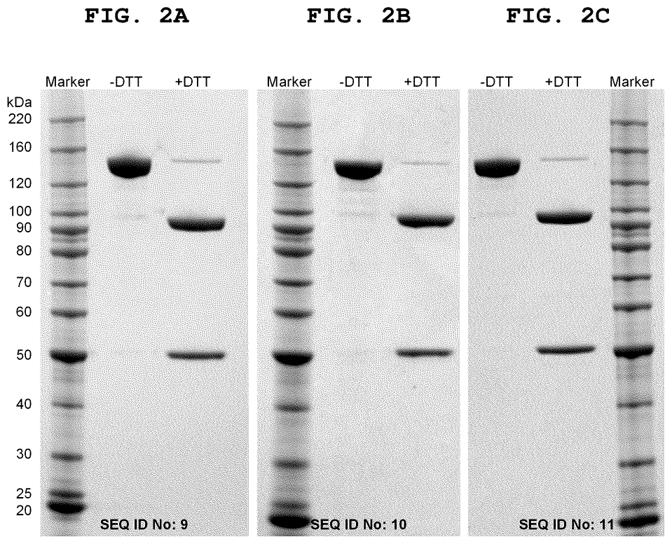

BoNT/AB chimera 1, 2 and 3A which have a C-terminal His₁₀ tag and E1191M/S1199Y double mutation were purified as described in Example 1 (FIG. 2) and tested for functional activity.

### Rat Spinal Cord Neurons Snap-25 Cleavage Assay

Primary cultures of rat spinal cord neurons (SCN) were prepared and grown, for 3 weeks, in 96 well tissue culture plates (as described in: Masuyer et al., 2011, J. Struct. Biol. Structure and activity of a functional derivative of Clostridium botulinum neurotoxin B; and in: Chaddock et al., 2002, Protein Expr. Purif Expression and purification of catalytically active, non-toxic endopeptidase derivatives of Clostridium botulinum toxin type A). Serial dilutions of BoNT/AB were prepared in SCN feeding medium. The growth medium from the wells to be treated was collected and filtered (0.2 μm filter). 125 μL of the filtered medium was added back to each test well. 125 μL of diluted toxin was then added to the plate (triplicate wells). The treated cells were incubated at 37° C., 10% CO₂, for 24±1 h).

### Analysis of BoNT Activity Using the SNAP-25 Cleavage Assay

Following treatment, BoNT was removed and cells were washed once in PBS (Gibco, UK). Cells were lysed in 1×NUPAGE™ lysis buffer (Life Technologies) supplemented with 0.1 M dithiothreitol (DTT) and 250 units/mL BENZONASE® nuclease (Sigma). Lysate proteins were separated by SDS-PAGE and transferred to nitrocellulose membranes. Membranes were probed with a primary antibody specific for SNAP-25 (Sigma #S9684) which recognizes uncleaved SNAP-25 as well as SNAP-25 cleaved by the BoNT/A endopeptidase. The secondary antibody used was an HRP-conjugated anti-rabbit IgG (Sigma #A6154). Bands were detected by enhanced chemiluminescence and imaged using a pXi6 Access (Synoptics, UK). The intensity of bands was determined using GENETOOLS® software (Syngene, Cambridge. UK) and the percentage of SNAP-25 cleaved at each concentration of BoNT calculated. Data were fitted to a 4-parameter logistic equation and pEC₅₀ calculated using GRAPHPAD PRISM® version 6 (GraphPad).

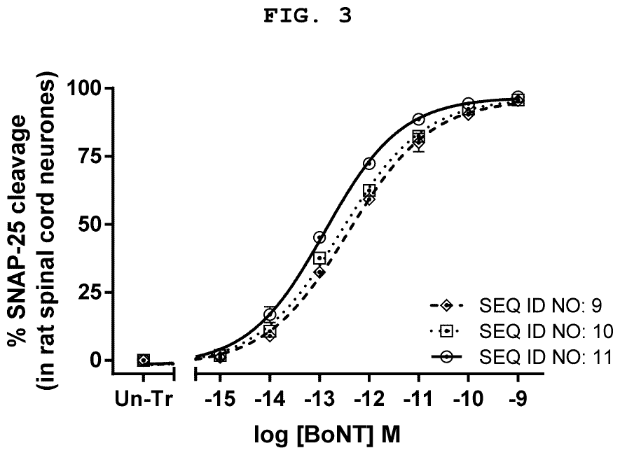

Table 4 below provides the pEC₅₀ values determined for Chimera 1, 2 and 3A in the rat SCN SNAP-25 cleavage assay. These results show that the three BoNT/AB chimeras retained the ability to enter rat spinal cord neurons and cleave their target substrate. However, chimera 3A was more potent than chimera 1 and 2 in this assay (see also FIG. 3).

TABLE 4

| | pEC₅₀ ± SEM |
|---|---|
| Chimera 1 | 12.42 ± 0.04 |
| Chimera 2 | 12.57 ± 0.01 |
| Chimera 3A | 12.89 ± 0.04 |

### Digit Abduction Scoring (DAS) Assay

The method to measure the activity of BoNT/AB chimera 1, 2 and 3A in the DAS assay is based on the startled response toe spreading reflex of mice, when suspended briefly by the tail. This reflex is scored as Digit Abduction Score (DAS) and is inhibited after administration of BoNT into the gastrocnemius-soleus muscles of the hind paw. Mice are suspended briefly by the tail to elicit a characteristic startled response in which the animal extends its hind limb and abducts its hind digits. (Aoki et al. 1999, Eur. J. Neurol.; 6 (suppl. 4) S3-S10)

On the day of injection, mice were anaesthetized in an induction chamber receiving isoflurane 3% in oxygen. Each mouse received an intramuscular injection of BoNT/AB chimera or vehicle (phosphate buffer containing 0.2% gelatine) in the gastrocnemius-soleus muscles of the right hind paw.

Following neurotoxin injection, the varying degrees of digit abduction were scored on a scale from zero to four, where 0=normal and 4=maximal reduction in digit abduction and leg extension. ED50 was determined by nonlinear adjustment analysis using average of maximal effect at each dose. The mathematical model used was the 4 parameters logistic model.

DAS was performed every 2 hours during the first day after dosing; thereafter it was performed 3 times a day for 4 days.

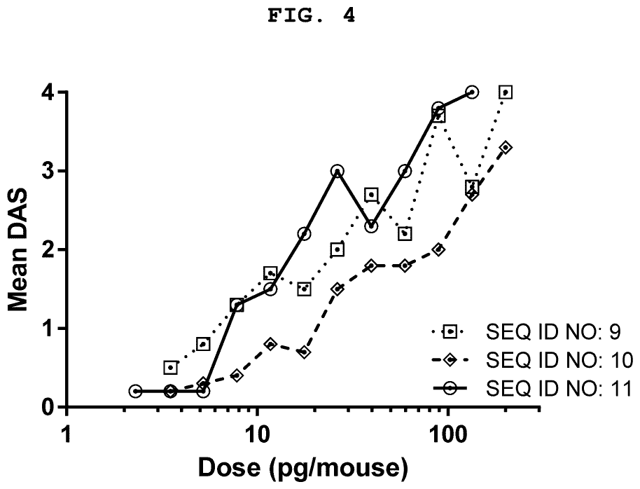

FIG. 4 shows the fitted curves for chimera 1, 2 and 3A (SEQ ID NO: 9, 10 and 11 respectively). The chimera 3A curve is shifted to the left, meaning lower doses of chimera 3A achieved a similar DAS response compared to chimera 1 and 2, therefore showing that chimera 3A is more potent than the others in the mouse DAS assay; see also the table below (table 5) that provides the values for the calculated ED50 and the dose leading to DAS 4 (highest score) for each chimera.

Table 5 below provides the ED₅₀ and DAS 4 doses determined for recombinant BoNT/A1 (rBoNT/A1) and chimeras 1, 2 and 3A in the mouse DAS assay. These results show that of the three chimeras, chimera 3A has the highest in vivo potency in inducing muscle weakening.

Studies shown in FIG. 4 and table 5 were performed in mice obtained from Charles River laboratories.

TABLE 5

| | ED₅₀ | |
|---|---|---|
| | (pg/ | DAS 4 dose |
| | mouse) | (pg/mouse) |
| rBoNT/A1 | 1 | 5 |
| Chimera 1 | 23 | 200 |
| Chimera 2 | 89 | >300 |
| Chimera 3A | 18 | 133 |

### Example 4—Comparison of BoNT/AB Chimera 3B, 3C and BoNT/A1

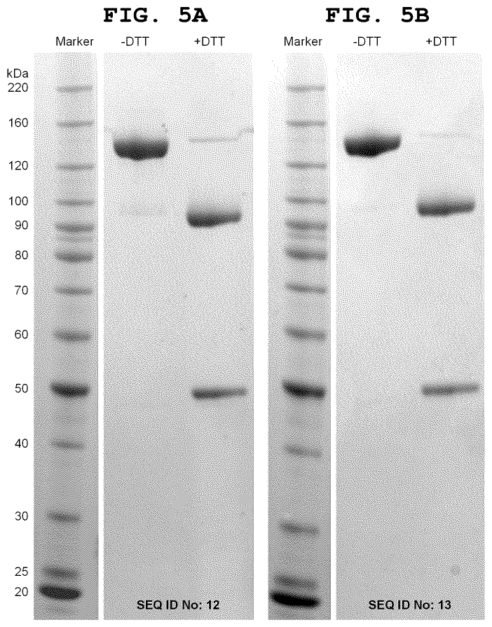

Untagged BoNT/AB chimera 3B and 3C, respectively with and without the presence of the E1191M/S1199Y double mutation (SEQ ID NO: 12 and 13) were purified as described in Example 1 (FIG. 5), and tested for functional activity using recombinant BoNT/A1 (SEQ ID NO: 1) as a reference.

### Human Pluripotent Stem Cells Snap-25 Cleavage Assay

Cryopreserved PERI.4U-cells were purchased from Axiogenesis (Cologne, Germany). Thawing and plating of the cells were performed as recommended by the manufacturer. Briefly, cryovials containing the cells were thawed in a water bath at 37° C. for 2 minutes. After gentle resuspension the cells were transferred to a 50 mL tube. The cryovial was washed with 1 mL of PERI.4U® thawing medium supplied by the manufacturer and the medium was transferred drop-wise to the cell suspension to the 50 mL tube, prior to adding a further 2 mL of PERI.4U® thawing medium drop-wise to the 50 mL tube. Cells were then counted using a hemocytometer. After this, a further 6 mL of PERI.4U® thawing medium was added to the cell suspension. A cell pellet was obtained by centrifugation at 260×g (e.g. 1,100 RPM) for 6 minutes at mom temperature. Cells were then resuspended in complete PERI.4U® culture medium supplied by the manufacturer. Cells were plated at a density of 50.000 to 150,000 cells per cm² on cell culture plates coated with poly-L-ornithine and laminin. Cells were cultured at 37° C. in a humidified CO₂ atmosphere, and medium was changed completely every 2-3 days during culture.

For toxin treatment, serial dilutions of BoNTs were prepared in PERI.4U culture medium. The medium from the wells to be treated was collected and filtered (0.2 μm filter). 125 μL of the filtered medium was added back to each test well. 125 μL of diluted toxin was then added to the plate (triplicate wells). The treated cells were incubated at 37° C. 10% CO₂, for 48±1 h).

### Analysis of BoNT Activity Using the SNAP-25 Cleavage Assay

Following treatment. BoNT was removed and cells were washed once in PBS (Gibco. UK). Cells were lysed in 1×NUPAGE™ lysis buffer (Life Technologies) supplemented with 0.1 M dithiothreitol (DTT) and 250 units/mL BENZONASE® nuclease (Sigma). Lysate proteins were separated by SDS-PAGE and transferred to nitrocellulose membranes. Membranes were probed with a primary antibody specific for SNAP-25 (Sigma #S9684) which recognizes uncleaved SNAP-25 as well as SNAP-25 cleaved by the BoNT/A endopeptidase. The secondary antibody used was an I-IRP-conjugated anti-rabbit IgG (Sigma #A6154). Bands were detected by enhanced chemiluminescence and imaged using a pXi6 Access (Synoptics. UK). The intensity of bands was determined using GENETOOLS® software (Syngene, Cambridge, UK) and the percentage of SNAP-25 cleaved at each concentration of BoNT calculated. Data were fitted to a 4-parameter logistic equation and pEC₅₀ calculated using GRAPHPAD PRISM® version 6 (GraphPad).

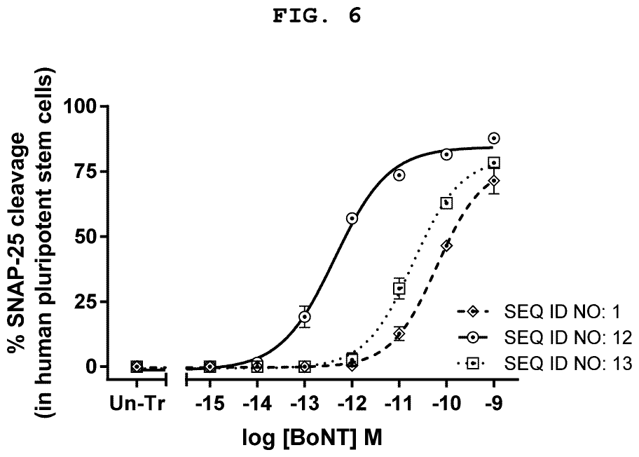

FIG. 6 shows that chimera 3B and 3C displayed greater potency than rBoNT/A1 in cleaving SNAP-25 in induced human pluripotent stem cells but the former significantly more so. This can be explained by the double mutation which increases the affinity of chimera 3B for the human synaptotagmin II protein receptor present in these cells (FIG. 6, table 6).

TABLE 6

| | pEC₅₀ ± SEM |
|---|---|
| rBoNT/A1 | 10.21 ± 0.05 |
| Chimera 3B | 12.38 ± 0.06 |
| Chimera 3C | 10.72 ± 0.08 |

### Digit Abduction Scoring (Das) Assay—Safety Ratio

The method to measure the activity of BoNTs in the DAS assay is based on the startled response toe spreading reflex of mice, when suspended briefly by the tail. This reflex is scored as Digit Abduction Score (DAS) and is inhibited after administration of BoNT into the gastrocnemius-soleus muscles of the hind paw. Mice are suspended briefly by the tail to elicit a characteristic startled response in which the animal extends its hind limb and abducts its hind digits. (Aoki et al. 1999, Eur. J. Neurol.; 6 (suppl. 4) S3-S10)

On the day of injection, mice were anaesthetized in an induction chamber receiving isoflurane 3% in oxygen. Each mouse received an intramuscular injection of BoNT or vehicle (phosphate buffer containing 0.2% gelatine) in the gastrocnemius-soleus muscles of the right hind paw.

Following neurotoxin injection, the varying degrees of digit abduction were scored on a scale from zero to four, where 0=normal and 4=maximal reduction in digit abduction and leg extension. ED50 was determined by nonlinear adjustment analysis using average of maximal effect at each dose. The mathematical model used was the 4 parameters logistic model.

DAS was performed every 2 hours during the first day after dosing; thereafter it was performed 3 times a day for 4 days for all doses. Animals of the groups injected with vehicle and the lowest dose that induced during the first four days of injection a DAS of 4 were thereafter monitored until complete recovery of the muscle weakness to a DAS of 0 (no observed muscle weakness).

For calculation of the safety ratio all animals were weighed the day before toxin injection (DO) and thereafter once daily throughout the duration of the study. The average body weight, its standard deviation, and the standard error mean were calculated daily for each dose-group. To obtain the safety ratio for a BoNT (−10% ΔBW/ED₅₀), the dose at which at any time during the study the average weight of a dose-group was lower than 10% of the average weight at DO of that same dose-group was divided by the ED₅₀ for the BoNT studied. The lethal dose was defined as the dose at which one or more of the animals within that dose-group died.

FIG. 7 shows the duration of muscle weakening over time in the mouse digit abduction scoring assay for rBoNT/A1, chimera 3B and chimera 3C (SEQ ID NO: 1, 12 and 13), showing that the chimera has longer duration of action.

Table 7 below provides the ED₅₀ and DAS 4 doses determined for rBoNT/A1 and chimeras 3B and 3C in the mouse DAS assay. The table also provide the total duration of action for the DAS 4 dose until complete recovery of the muscle weakness to a DAS of 0 (no observed muscle weakness). In addition, the table shows the mouse lethal dose and the safety ratio (−10% ABW/ED₅₀), as defined in the text above. In comparison to rBoNT/A1, chimeras 3B and 3C have longer duration of action, a better safety ratio, and a higher lethal dose. Studies shown in FIG. 7 and table 7 were performed in mice obtained from Janvier laboratories.

TABLE 7

| | ED₅₀ | | Total | | |
|---|---|---|---|---|---|
| | (DAS 2) | DAS 4 | duration of | Mouse | Safety |
| | Dose | dose | action (day) | lethal | ratio |
| | (pg/ | (pg/ | with lowest | dose | (−10% |
| | mouse) | mouse) | DAS 4 dose | (pg) | ΔBW/ED₅₀) |
| rBoNT/Al | 0.9 | 2.3 | 29 | 18 | 4.5 |
| Chimera 3B | 8.0 | 89 | 42 | 200 | 14.1 |
| Chimera 3C | 5.0 | 26 | 42 | 8.9 | 7.4 |

### Example 5—Expression and Purification of BoNT/BC Chimera and Confirmation of Functional Activity

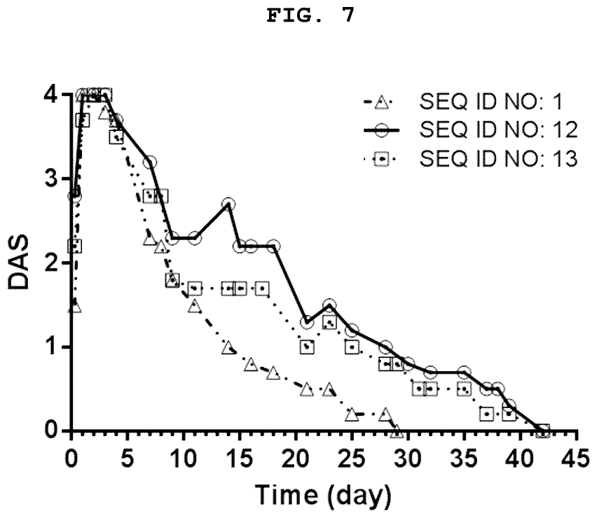

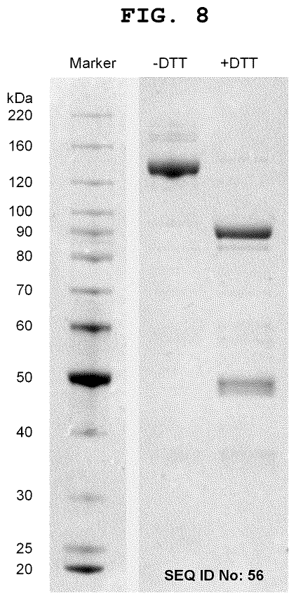

BoNT/BC chimera 4 (SEQ ID NO: 56) was cloned, expressed and purified as described in Example 2 except for the use of a different expression cell strain (BL21) and proteolytic cleavage with trypsin rather than endoproteinase Lys-C (FIG. 8).

TABLE 8
chimeric BoNT/BC construct

| Molecule | SEQ ID NO | Sequence |
|---|---|---|
| Chimera 4 | 56 | B1:1-859 + C1:868-1291 |

This chimera was tested for functional activity in a VAMP-2 cleavage assay.

### Rat Cortical Neuron Vamp-2 Cleavage Assay

Rat cortical neurons were prepared and maintained on poly-L-ornithine (PLO) coated 96-well plates at a density of 20000 cells/well in 125 μL NEUROBASAL® media containing 2% B27 supplement, 0.5 mM GLUTAMAX™ supplement, 1% foetal bovine serum (FBS) and 100 U/mL penicillin/streptomycin, at 37° C. in a humidified atmosphere containing 5% CO₂. A further 125 μL NEUROBASAL® medium containing 2% B27, 0.5 mM GLUTAMAX™ supplement was added on DIV 4. Cells were maintained by replacement of half the medium every 3-4 days. On DIV 11, 1.5 μM cytosine β-D-arabinofuranoside (AraC) was added to the medium to prevent proliferation of non-neuronal cells. Cortical neurons at DIV 19-21 were treated with a concentration range of BoNT (30 fM-3 nM) for 24 hours at 37° C.

### Analysis of BoNT Activity Using the VAMP-2 Cleavage Assay

Cells were briefly washed in assay medium (NEUROBASAL® media w/o phenol red, 2% B27, 0.5 mM GLUTAMAX™ supplement, 10 μM TFB-TBOA ((3S)-3-[[3-[[4-(Trifluoromethyl) benzoyl]amino]phenyl]methoxy]-L-aspartic acid, before lysis in 100 μL lysis buffer (NUPAGE® LDS sample buffer, 1 mM DTT and 1:500 BENZONASE® nuclease) and heated at 90° C. for 5 minutes. 15 μL lysates were run in 12% Bis-Tris gels at 200 V for 50 minutes with MES buffer. Proteins were transferred onto nitrocellulose membranes via a TRANS-BLOT® TURBO™ transfer system (Biorad) using the low MW program. Membranes were blocked with 5% low fat milk in PBST and then probed with rabbit anti-VAMP-2 (Abcam ab3347, 1:1000) primary antibody and then HRP-conjugated anti-rabbit secondary antibody (Sigma #A6154). Membranes were developed with SUPERSIGNAL® West Dura chemiluminescent substrate and visualized using a SYNGENE® PXi system. Band densitometry was analyzed using GENETOOLS® software (Syngene) and VAMP-2 percentage cleavage at each concentration of BoNT was determined relative to the control wells. Data was fit to a 4-parameter logistic equation and the pEC₅₀ calculated using GRAPHPAD PRISM® software (GraphPad).

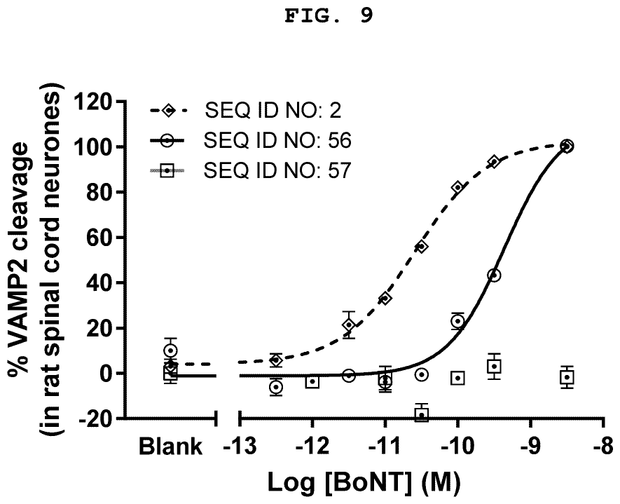

FIG. 9 shows that chimera 4 is able to bind to rat spinal cord neurones, translocate into the cytoplasm, and specifically cleave its substrate VAMP-2. As a point of reference, this chimera is clearly functional compared an inactive recombinant BoNT/B1 molecule (having a double mutation at E231Q and H234Y, and also referred herein as BoNT/B1(0)) (SEQ ID NO: 57), and is almost as active as the native BoNT/B1 molecule (SEQ ID NO: 2) (Table 9). This may be explained by the high affinity binding of BoNT/B to synaptotagmin and various gangliosides present on the rat cell surface, whereas the binding domain of C in chimera 4, is only known to bind with lower affinity to gangliosides. This is supported by data from the light chain protease activity assay, as shown further below.

TABLE 9

| | pEC50 ± SEM |
|---|---|
| | (VAMP-2 cleavage |
| | assay in rat cortical cells) |
| native | 10.60 ± 0.06 |
| BoNT/B1 | |
| Chimera 4 | 9.36 ± 0.15 |
| BoNT/B1(0) | inactive |

### Light Chain Protease Activity Assay

The light chain activity of serotype B was assessed using the BOTEST® (BioSentinel A1009) cell-free assay according to the manufacturer's instructions. For example, BoNTs were diluted to 1.39 nM in BOTEST® Reaction Buffer (50 mM HEPES-NaOH, 5 mM NaCl, 10 μM ZnCl₂, 0.1% Tween-20, 0.1 mg/ml BSA, pH 7.1) and reduced with 5 mM DTT for 30 minutes at room temperature. VAMP-2 peptide reporter (CFP-VAMP-2(33-94)-YFP in 50 mM HEPES-NaOH. 10 mM NaCl, 15% glycerol) at a final concentration of 200 nM was combined with a concentration range of BoNTs (500 fM-1.25 nM, final) in black MAXISORP® plates (Nunc) in a final assay volume of 100 μL/well. The plates were sealed and incubated at 30° C. for 18 hours away from light. The loss of CFP to YFP FRET fluorescence at 528 nm and gain of GFP fluorescence at 485 nm following excitation at 440 nm was measured using a BIOTEK® SYNGERY® HT plate reader. The fluorescence emission ratio of uncleaved:cleaved reporter substrate at each BoNT concentration was tit to a 4-parameter logistic equation and the pEC₅₀ calculated using GRAPHPAD PRISM® software.

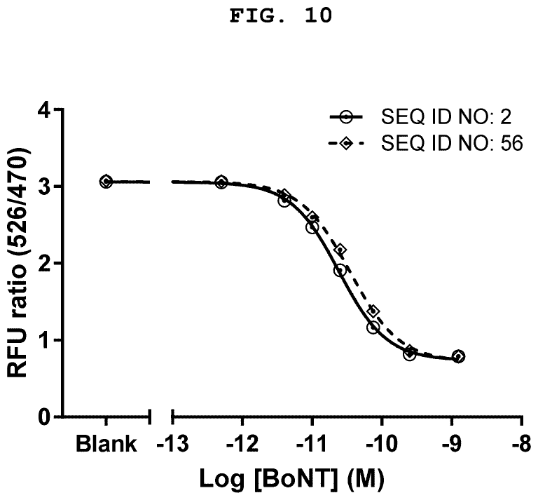

The light chain protease activity assay confirms that the light chain of chimera 4 is as active as the one of native BoNT/B1 (see FIG. 10, and Table 10), and therefore that, as explained above, the results of the VAMP-2 cleavage assay may be explained by the fact that BoNT/B has a higher affinity to synaptotagmin and various gangliosides present on the rat cell surface, compared to the binding domain of C in chimera 4 which only binds to gangliosides.

TABLE 10

| | pEC50 ± SEM |
|---|---|
| | (VAMP-2 peptide |
| | cleavage assay in vitro) |
| native | 10.62 ± 0.01 |
| BoNT/B1 | |
| Chimera 4 | 10.46 ± 0.01 |
| BoNT/B1(0) | NA |

1. A chimeric neurotoxin comprising a LH_{N} domain from a first neurotoxin covalently linked to a H_{C} domain from a second neurotoxin,
wherein said first and second neurotoxins are different,
wherein the C-terminal amino acid residue of said LH_{N} domain corresponds to the first amino acid residue of the 3₁₀ helix separating the LH_{N} and H_{C} domains in said first neurotoxin, and
wherein the N-terminal amino acid residue of said H_{C} domain corresponds to the second amino acid residue of the 3₁₀ helix separating the LH_{N} and H_{C} domains in said second neurotoxin.

2. A chimeric neurotoxin according to claim 1, wherein said first neurotoxin is a Botulinum Neurotoxin (BoNT) serotype A, serotype B, serotype C, serotype D, serotype E, serotype F or serotype G or a Tetanus Neurotoxin (TeNT), and wherein said second neurotoxin is a Botulinum Neurotoxin (BoNT) serotype A, serotype B, serotype C, serotype D, serotype E, serotype F or serotype G or a Tetanus Neurotoxin (TeNT).

3. A chimeric neurotoxin according to claim 1 or 2, wherein said LH_{N} domain from a first neurotoxin corresponds to:
amino acid residues 1 to 872 of BoNT/A1,
amino acid residues 1 to 859 of BoNT/B1,
amino acid residues 1 to 867 of BoNT/C1,
amino acid residues 1 to 863 of BoNT/D,
amino acid residues 1 to 846 of BoNT/E1,
amino acid residues 1 to 865 of BoNT/F1,
amino acid residues 1 to 864 of BoNT/G, or
amino acid residues 1 to 880 of TeNT.
and wherein said H_{C} domain from a second neurotoxin corresponds to:
amino acid residues 873 to 1296 of BoNT/A1,
amino acid residues 860 to 1291 of BoNT/B1,
amino acid residues 868 to 1291 of BoNT/C1,
amino acid residues 864 to 1276 of BoNT/D, amino acid residues 847 to 1251 of BoNT/E1,
amino acid residues 866 to 1275 of BoNT/F1,
amino acid residues 865 to 1297 of BoNT/G, or
amino acid residues 881 to 1315 of TeNT.

4. A chimeric neurotoxin according to claim 1, 2 or 3, wherein said first neurotoxin is a BoNT/A and wherein said second neurotoxin is a BoNT/B.

5. A chimeric neurotoxin according to claim 4, wherein said first neurotoxin is a BoNT/A and wherein said second neurotoxin is a BoNT/B.

6. A chimeric neurotoxin according to claim 5, wherein said LH_{N} domain from a first neurotoxin corresponds to amino acid residues 1 to 872 of BoNT/A1 and wherein said H_{C} domain from a second neurotoxin corresponds to amino acid residues 860 to 1291 of BoNT/B1.

7. A chimeric neurotoxin according to claim 4, 5 or 6, wherein said H_{C} domain from a BoNT/B neurotoxin comprises at least one amino acid residue substitution, addition or deletion in the H_{CC} subdomain which has the effect of increasing the binding affinity of the BoNT/B neurotoxin for the human Syt II receptor as compared to the natural BoNT/B sequence.

8. A chimeric neurotoxin according to claim 7, wherein said at least one amino acid residue substitution, addition or deletion in the H_{CC} subdomain comprises a substitution mutation selected from the group consisting of: V1118M; Y1183M; E1191M; E11911; E1191Q; E1191T; S1199Y; S1199F; S1199L; S1201V; E1191C, E1191V, E1191L, E1191Y, S1199W, S1199E, S1199H, W1178Y, W1178Q, W1178A, W1178S, Y1183C, Y1183P and combinations thereof

9. A chimeric molecule according to claim 7, wherein said at least one amino acid residue substitution, addition or deletion in the H_{CC} subdomain comprises two substitution mutations selected from the group consisting of: E1191M and S1199L, E1191M and S1199Y, E1191M and S1199F, E1191Q and S1199L, E1191Q and S1199Y, E1191Q and S1199F, E1191M and S1199W, E1191M and W1178Q, E1191C and S1199W, E1191C and S1199Y, E1191C and W1178Q, E1191Q and S1199W, E1191V and S1199W, E1191V and S1199Y, or E1191V and W1178Q.

10. A chimeric neurotoxin according to claim 9, wherein said two substitution mutations are E1191M and S1199Y.

11. A chimeric molecule according to claim 7, wherein said at least one amino acid residue substitution, addition or deletion in the H_{CC} subdomain comprises three substitution mutations which are E1191M, S1199W and W1178Q.

12. A chimeric neurotoxin according to claim 1, 2 or 3, wherein said first neurotoxin is a BoNT/B and wherein said second neurotoxin is a BoNT/C.

13. A chimeric neurotoxin according to claim 12, wherein said first neurotoxin is a BoNT/B1 and wherein said second neurotoxin is a BoNT/C1.

14. A chimeric neurotoxin according to claim 13, wherein said LH_{N} domain from a first neurotoxin corresponds to amino acid residues 1 to 859 of BoNT/B1 and wherein said H_{C} domain from a second neurotoxin corresponds to amino acid residues 868 to 1291 of BoNT/C1.

15. A nucleotide sequence encoding a chimeric neurotoxin according to any one of claim 1 to 14.

16. A vector comprising a nucleotide sequence according to claim 15.

17. A cell comprising a nucleotide sequence according to claim 15 or a vector according to claim 16.

18. A pharmaceutical composition comprising a chimeric neurotoxin according to any one of claims 1 to 14.

19. A kit comprising a pharmaceutical composition of claim 18 and instructions for therapeutic or cosmetic administration of said composition to a subject in need thereof.

20. A method for producing a chimeric neurotoxin as defined in any one of claims 1 to 14, said method comprising the step of culturing a cell of claim 17, under conditions wherein said chimeric neurotoxin is produced.

21. A chimeric neurotoxin according to any one of claims 1 to 14 or pharmaceutical composition according to claim 18 for use in therapy.

22. A chimeric neurotoxin or pharmaceutical composition according to claim 21, for use in treating a condition associated with unwanted neuronal activity, for example a condition selected from the group consisting of spasmodic dysphonia, spasmodic torticollis, laryngeal dystonia, oromandibular dysphonia, lingual dystonia, cervical dystonia, focal hand dystonia, blepharospasm, strabismus, hemifacial spasm, eyelid disorder, cerebral palsy, focal spasticity and other voice disorders, spasmodic colitis, neurogenic bladder, anismus, limb spasticity, tics, tremors, bruxism, anal fissure, achalasia, dysphagia and other muscle tone disorders and other disorders characterized by involuntary movements of muscle groups, lacrimation, hyperhidrosis, excessive salivation, excessive gastrointestinal secretions, secretory disorders, pain from muscle spasms, headache pain, migraine and dermato logical conditions.

23. Non therapeutic use of a chimeric neurotoxin according to any one of claims 1 to 14 or of a pharmaceutical composition according to claim 18, for treating an aesthetic or cosmetic condition.
